SEQUENCE LISTING (7 sequences)

SEQ ID NO: 1 - DNA, 20 bp, artificial
  primer
agagtttgatcctggctcag

SEQ ID NO: 2 - DNA, 19 bp, artificial
  primer
ggttaccttgttacgactt

SEQ ID NO: 3 - protein, 131 aa, Unknown
  Environmental sample
STGCTSGLDSVGYAVQLIREGSADVVIAGAADTPVSPIVVACFDAIKATTPRNDDPEHASRPFDGTRNGFVLAEGAAMFVLEEYEAAKRRGAHIYAEVGGYATRCNAYHMTGLKKDGREMAEAIRAALDEA

SEQ ID NO: 4 - protein, 132 aa, Streptomyces cyaneus
VSTGCTSGLDAVGYAFHTIEEGRADVCIAGASDSPISPITMACFDAIKATSPNNDDPEHASRPFDAHRDGFVMGEGAAVLVLEELEHARARGAHVYCEIGGYATFGNAYHMTGLTSEGLEMARAIDVALDHA

SEQ ID NO: 5 - protein, 132 aa, Streptomyces halstedii
VSTGCTSGLDAVGYAYHAIAEGRADVCLAGASDSPISPITMACFDAIKATSPSNDDPEHASRPFDARRNGFVMGEGGAVLVLEELEHARARGADVYCELAGYATFGNAHHMTGLTREGLEMARAIDTALDMA

SEQ ID NO: 6 - protein, 132 aa, Streptomyces peucetius
VSAGCTSGIDSIGYACELIREGTVDAMVAGGVDAPIAPITVACFDAIRATSDHNDTPETASRPFSRSRNGFVLGEGGAIVVLEEAEAAVRRGARIYAEIGGYASRGNAYHMTGLRADGAEMAAAITAALDEA

SEQ ID NO: 7 - protein, 132 aa, Escherichia coli
IATACTSGVHNIGHAARIIAYGDADVMVAGGAEKASTPLGVGGFGAARALSTRNDNPQAASRPWDKERDGFVLGDGAGMLVLEEYEHAKKRGAKIYAELVGFGMSSDAYHMTSPPENGAGAALAMANALRDA

(19) United States
(12) Patent Application Publication
(10) Pub. No.: US 2006/0094033 A1
(43) Pub. Date: May 4, 2006

(54) Screening methods and libraries of trace amounts of DNA from uncultivated microorganisms

(76) Inventors: Carl Abulencia, San Diego, CA (US); Martin Keller, San Diego, CA (US)

Correspondence Address: DIVERSA C/O MOFO S.D., 12531 HIGH BLUFF DRIVE, SUITE 100, SAN DIEGO, CA, 92130-2040, US

(21) Appl. No.: 11/134,852

(22) Filed: May 20, 2005

Related U.S. Application Data
(60) Provisional application No. 60/573,473, filed on May 21, 2004.

(51) Int. Cl. C12Q 1/68; C12P 19/34

(52) U.S. Cl. 435/6; 435/91.2

(57) ABSTRACT

The invention provides methods for making a gene library from trace amounts of DNA derived from a plurality of species of organisms comprising obtaining trace amounts of cDNA, gDNA, or genomic DNA fragments from a plurality of species of organisms, amplifying the DNA so obtained, and ligating the DNA to a DNA vector to generate a library of constructs in which genes are contained in the DNA. The invention also provides methods for screening clones having DNA recovered from trace amounts of DNA derived from a plurality of species of uncultivated organisms. The invention also provides methods for identifying and enriching for a polynucleotide encoding an activity of interest.

### FIELD OF THE INVENTION

This invention relates to the field of preparing and screening libraries of clones containing DNA derived from trace amounts of microbially derived DNA.

### BACKGROUND OF THE INVENTION

There is a critical need in the chemical industry for efficient catalysts for the practical synthesis of optically pure materials; enzymes can provide the optimal solution. All classes of molecules and compounds that are utilized in both established and emerging chemical, pharmaceutical, textile, food and feed, detergent markets must meet stringent economical and environmental standards. The synthesis of polymers, pharmaceuticals, natural products and agrochemicals is often hampered by expensive processes which produce harmful byproducts and which suffer from low enantioselectivity. Enzymes have a number of remarkable advantages that can overcome these problems in catalysis: they act on single functional groups, they distinguish between similar functional groups on a single molecule, and they distinguish between enantiomers. Moreover, they are biodegradable and function at very low mole fractions in reaction mixtures. Because-of their chemo-, regio- and stereospecificity, enzymes present a unique opportunity to optimally achieve desired selective transformations. These are often extremely difficult to duplicate chemically, especially in single-step reactions. The elimination of the need for protection groups, selectivity, the ability to carry out multi-step transformations in a single reaction vessel, along with the concomitant reduction in environmental burden, has led to the increased demand for enzymes in chemical and pharmaceutical industries. Enzyme-based processes have been gradually replacing many conventional chemical-based methods. A current limitation to more widespread industrial use is primarily due to the relatively small number of commercially available enzymes. Only ˜300 enzymes (excluding DNA modifying enzymes) are at present commercially available from the >3000 non DNA-modifying enzyme activities thus far described.

The use of enzymes for technological applications also may require performance under demanding industrial conditions. This includes activities in environments or on substrates for which the currently known arsenal of enzymes was not evolutionarily selected. Enzymes have evolved by selective pressure to perform very specific biological functions within the milieu of a living organism, under conditions of mild temperature, pH and salt concentration. For the most part, the non-DNA modifying enzyme activities thus far described have been isolated from mesophilic organisms, which represent a very small fraction of the available phylogenetic diversity. The dynamic field of biocatalysis takes on a new dimension with the help of enzymes isolated from microorganisms that thrive in extreme environments. Such enzymes must function at temperatures above 100° C. in terrestrial hot springs and deep sea thermal vents, at temperatures below 0° C. in arctic waters, in the saturated salt environment of the Dead Sea, at pH values around 0 in coal deposits and geothermal sulfur-rich springs, or at pH values greater than 11 in sewage sludge. Enzymes obtained from these extremophilic organisms open a new field in biocatalysis.

In addition to the need for new enzymes for industrial use, there has been a dramatic increase in the need for bioactive compounds with novel activities. This demand has arisen largely from changes in worldwide demographics coupled with the clear and increasing trend in the number of pathogenic organisms that are resistant to currently available antibiotics. For example, while there has been a surge in demand for antibacterial drugs in emerging nations with young populations, countries with aging populations, such as the US, require a growing repertoire of drugs against cancer, diabetes, arthritis and other debilitating conditions. The death rate from infectious diseases has increased 58% between 1980 and 1992 and it has been estimated that the emergence of antibiotic resistant microbes has added in excess of $30 billion annually to the cost of health care in the US alone. (Adams et al., Chemical and Engineering News, 1995; Amann et al., Microbiological Reviews, 59, 1995). As a response to this trend pharmaceutical companies have significantly increased their screening of microbial diversity for compounds with unique activities or specificities.

There are several common sources of lead compounds (drug candidates), including natural product collections, synthetic chemical collections, and synthetic combinatorial chemical libraries, such as nucleotides, peptides, or other polymeric molecules. Each of these sources has advantages and disadvantages. The success of programs to screen these candidates depends largely on the number of compounds entering the programs, and pharmaceutical companies have to date screened hundred of thousands of synthetic and natural compounds in search of lead compounds. Unfortunately, the ratio of novel to previously discovered compounds has diminished with time. The discovery rate of novel lead compounds has not kept pace with demand despite the best efforts of pharmaceutical companies. There exists a strong need for accessing new sources of potential drug candidates.

The majority of bioactive compounds currently in use are derived from soil microorganisms. Many microbes inhabiting soils and other complex ecological communities produce a variety of compounds that increase their ability to survive and proliferate. These compounds are generally thought to be nonessential for growth of the organism and are synthesized with the aid of genes involved in intermediary metabolism hence their name—“secondary metabolites”. Secondary metabolites that influence the growth or survival of other organisms are known as “bioactive” compounds and serve as key components of the chemical defense arsenal of both micro- and macroorganisms. Humans have exploited these compounds for use as antibiotics, antiinfectives and other bioactive compounds with activity against a broad range of prokaryotic and eukaryotic pathogens. Approximately 6,000 bioactive compounds of microbial origin have been characterized, with more than 60% produced by the gram-positive soil bacteria of the genus Streptomyces. (Barnes et al., Proc. Nat. Acad. Sci. U.S.A., 91, 1994). Of these, at least 70 are currently used for biomedical and agricultural applications. The largest class of bioactive compounds, the polyketides, include a broad range of antibiotics, immunosuppressants and anticancer agents which together account for sales of over $5 billion per year.

Despite the seemingly large number of available bioactive compounds, it is clear that one of the greatest challenges facing modem biomedical science is the proliferation of antibiotic resistant pathogens. Because of their short generation time and ability to readily exchange genetic information, pathogenic microbes have rapidly evolved and disseminated resistance mechanisms against virtually all classes of antibiotic compounds. For example, there are virulent strains of the human pathogens Staphylococcus and Streptococcus that can now be treated with but a single antibiotic, vancomycin, and resistance to this compound will require only the transfer of a single gene, vanA, from resistant Enterococcus species for this to occur. (Bateson et al., System. Appl. Microbiol, 12, 1989). When this crucial need for novel antibacterial compounds is superimposed on the growing demand for enzyme inhibitors, immunosuppressants and anti-cancer agents it becomes readily apparent why pharmaceutical companies have stepped up their screening of microbial diversity for bioactive compounds with novel properties.

It has been estimated that to date less than one percent of the world's organisms have been cultured. It has been suggested that a large fraction of this diversity thus far has been unrecognized due to difficulties in enriching and isolating microorganisms in pure culture. Therefore, it has been difficult or impossible to identify or isolate valuable proteins, from these samples. These limitations suggest the need for alternative approaches to obtain genomic DNA and characterize the physiological and metabolic potential, i.e. activities of interest of as-yet uncultivated microorganisms, which to date have been characterized solely by analyses of PCR amplified rRNA gene fragments, clonally recovered from mixed assemblage nucleic acids.

Current methods of PCR amplification involve the use of two primers which hybridize to the regions flanking a nucleic acid sequence of interest such that DNA replication initiated at the primers will replicate the nucleic acid sequence of interest. By separating the replicated strands from the template strand with a denaturation step, another round of replication using the same primers can lead to geometric amplification of the nucleic acid sequence of interest. A variant of PCR amplification, termed whole genome PCR, involves the use of random or partially random primers to amplify the entire genome of an organism in the same PCR reaction. This technique relies on having a sufficient number of primers of random or partially random sequence such that pairs of primers will hybridize throughout the genomic DNA at moderate intervals. Replication initiated at the primers can then result in replicated strands overlapping sites where another primer can hybridize. By subjecting the genomic sample to multiple amplification cycles, the genomic sequences will be amplified.

However, PCR amplification has the disadvantage that the amplification reaction cannot proceed continuously and must be carried out by subjecting the nucleic acid sample to multiple cycles in a series of reaction conditions. These reaction conditions often rely on cycling at high temperatures, which may cause degradation of long pieces of DNA. The multiple random amplification cycles, as used in whole genome PCR, can also be a disadvantage because of potential amplification of the products made in previous cycles, instead of randomly amplifying the original sequence. Further, enzymes currently used in PCR amplification cannot proceed along long genomic pieces of DNA (i.e., 40 kb and larger). Thus, amplification of entire genomes for use in large insert libraries is not possible using standard techniques.

Recent developments provide new methods of amplification of target nucleic acid sequences and whole genomes or other highly complex nucleic acid samples. U.S. Pat. No. 6,124,120, herein incorporated by reference, teaches Whole Genome Strand Displacement Amplification, in which a set of primers having random or partially random nucleotide sequences is used to randomly prime a sample of genomic nucleic acid. By choosing a sufficiently large set of primers of random or mostly random sequence, the primers in the set will be collectively, and randomly, complementary to nucleic acid sequences distributed throughout nucleic acid in the sample. Amplification proceeds by replication with a processive polymerase initiated at each primer and continuing until spontaneous termination. Similarly, U.S. Pat. No. 5,001,050, herein incorporated by reference, teaches amplification methods of very large fragments of DNA using Rolling Circle Amplification for circular templates. However, the teachings of both inventions disclose methods of amplifying nucleic acid from a single organism. Our inventors realized that applying these techniques to samples of large strands of DNA from a plurality of species invites potential under representation of all of the genomes present in the sample.

Previously, whole genome amplification from the gDNA of an isolate has been performed on Xylella fastidiosa using (RCA) on 1000 cells. (See Detter, et al., Isothermal Strand-Displacement Amplification Applications for High-Throughput Genomics, Gcnomics, Vol. 80, No. 6 (Decmeber 2002), incorporated by reference herein in its entirety.)

Methods for isothermal amplification of whole genomes were previously been described. (See Lage, et al., Whole Genome Analysis of Genetic Alterations in Small DNA Samples Using Hyperbranched Strand Displacement Amplification and Array-CGH, Genome Research, 13:294-307 (2003), herein incorporated by reference in its entirety.)

Therefore, the need exists for alternative approaches to obtain and amplify trace amounts of whole genomic DNA derived from at least one organism, and characterize the physiological and metabolic potential, i.e. activities of interest of as-yet uncultivated microorganisms from extreme and/or contaminated environments, clonally recovered from mixed assemblage nucleic acids.

### SUMMARY OF THE INVENTION

The present invention provides a novel approach to obtain and amplify trace amounts of whole genomic DNA derived from a plurality of organisms. In accordance with one aspect of the present invention, environmental samples that do not contain enough DNA for analysis by traditional methods are subject to multiple displacement amplification to enable the recovery of substantially the whole genomic DNA represented and to characterize as to physiological and metabolic potential.

More particularly, one aspect of the invention provides a process for making a gene library from trace amounts of DNA derived from a plurality of species of organisms comprising obtaining trace amounts of cDNA, gDNA, or genomic DNA fragments from a plurality of species of organisms, amplifying the cDNA, gDNA, or genomic DNA fragments, and ligating the cDNA, gDNA, or genomic DNA fragments to a DNA vector to generate a library of constructs in which genes are contained in the cDNA, gDNA, or genomic DNA fragments.

The organisms are uncultured organisms from environmental samples. The environmental sample may contain contaminated soil wherein only trace amounts of DNA exist. The organisms may be extremophiles such as thermophiles, hyperthermophiles, psychrophiles, phsychrotrophs, halophiles, alkalophiles, and acidophiles. In one aspect of this invention, the organisms comprise a mixture of terrestrial microorganisms or marine organisms, or a mixture of terrestrial microorganisms and marine microorganisms.

Another aspect of the invention provides a process of screening clones having DNA recovered from a plurality of species of uncultivated organisms having trace amounts of DNA for a specified protein, e.g. enzyme, activity which process comprises: screening for a specified protein, e.g. enzyme, activity in a library of clones prepared by: (i) recovering trace amounts of DNA from a DNA population derived from a plurality of species of uncultivated microorganisms; (ii) amplifying the trace amounts of DNA; and (iii) transforming a host with DNA to produce a library of clones which are screened for the specified protein, e.g. enzyme, activity.

The library is produced from DNA that is recovered without culturing of an organism, particularly where the DNA is recovered from an environmental sample containing organisms that are not or cannot be cultured and having trace amounts of DNA.

Preferably, the trace amounts of DNA are recovered without culturing of an organism, and are recovered from extreme and/or contaminated environmental samples containing organisms which are not or cannot be cultured.

In a preferred embodiment DNA is ligated into a vector, particularly wherein the vector further comprises expression regulatory sequences that can control and regulate the production of a detectable protein, e.g. enzyme, activity from the ligated DNA.

The f-factor (or fertility factor) in E. coli is a plasmid which effects high frequency transfer of itself during conjugation and less frequent transfer of the bacterial chromosome itself. To achieve and stably propagate large DNA fragments from mixed microbial samples, a particularly preferred embodiment is to use a cloning vector containing an f-factor origin of replication to generate genomic libraries that can be replicated with a high degree of fidelity. When integrated with DNA from a mixed uncultured environmental sample, this makes it possible to achieve large genomic fragments in the form of a stable “environmental DNA library.”

In another preferred embodiment, double stranded DNA obtained from the uncultivated DNA population is selected by: converting the double stranded genomic DNA into single stranded DNA; recovering from the converted single stranded DNA single stranded DNA which specifically binds, such as by hybridization, to a probe DNA sequence; and converting recovered single stranded DNA to double stranded DNA.

The probe may be directly or indirectly bound to a solid phase by which it is separated from single stranded DNA which is not hybridized or otherwise specifically bound to the probe.

The process can also include releasing single stranded DNA from said probe after recovering said hybridized or otherwise bound single stranded DNA and amplifying the single stranded DNA so released prior to converting it to double stranded DNA.

The invention also provides a process of screening clones having DNA from uncultivated microorganisms for a specified protein, e.g. enzyme, activity which comprises screening for a specified gene cluster protein product activity in the library of clones prepared by: (i) recovering DNA from a DNA population derived from a plurality of uncultivated microorganisms; (ii) amplifying the recovered DNA; and (iii) transforming a host with recovered DNA to produce a library of clones with the screens for the specified protein, e.g. enzyme, activity. In one aspect of this invention, the trace amounts of DNA are recovered from the microorganisms. In another aspect, very few cells of the microorganisms are available within the environmental sample.

The library is produced from gene cluster DNA that is recovered without culturing of an organism, particularly where the DNA gene clusters are recovered from an environmental sample containing organisms that are not or cannot be cultured and having trace amounts of DNA.

Preferably, the trace amounts of DNA are recovered without culturing of an organism, and are recovered from extreme and/or contaminated environmental samples containing organisms that are not or cannot be cultured.

Alternatively, double-stranded gene cluster DNA obtained from the uncultivated DNA population is selected by converting the double-stranded genomic gene cluster DNA into single-stranded DNA; recovering from the converted single-stranded gene cluster polycistron DNA, single-stranded DNA which specifically binds, such as by hybridization, to a polynucleotide probe sequence; and converting recovered single-stranded gene cluster DNA to double-stranded DNA.

In one aspect of the present invention, is provided a method for amplifying a DNA template from trace amounts of DNA derived from a plurality of species of organisms comprising: obtaining trace amounts of cDNA, gDNA, or genomic DNA fragments from a plurality of species of organisms; preparing a template from said cDNA, gDNA, or genomic DNA fragments; and amplifying the template.

In another aspect, the invention provides a method for amplifying a DNA template from trace amounts of DNA derived from a plurality of species of organisms comprising: obtaining trace amounts of cDNA, gDNA, or genomic DNA fragments from a plurality of species of organisms; preparing a circular template from said cDNA, gDNA, or genomic DNA fragments; and amplifying the template.

In another aspect, the invention provides a method for making a DNA template from trace amounts of DNA isolated from trace amounts of DNA from a mixed population of uncultivated cells comprising: encapsulating individually, in a microenvironment, a plurality of cells from a mixed population of uncultivated cells; creating a template from said cDNA, gDNA, or genomic DNA fragments; and amplifying the template.

The methods of the present invention also find use for DNA, including ancient DNA, forensic DNA, pre-fragmented, degraded DNA (UV, chemical, oxygen, peroxide, and photochemical exposure, among others).

These and other aspects of the present invention are described with respect to particular preferred embodiments and will be apparent to those skilled in the art from the teachings herein.

### BRIEF DESCRIPTION OF THE FIGURES

The following drawings are illustrative of embodiments of the invention and are not meant to limit the scope of the invention as encompassed by the claims.

FIG. 1 illustrates the protocol used in the cell sorting method of the invention to screen for a polynucleotide of interest, in this case using a (library excised into E. coli). The clones of interest are isolated by sorting.

FIG. 2 shows a microtiter plate where clones or cells are sorted in accordance with the invention. Typically one cell or cells grown within a microdroplet are dispersed per well and grown up as clones.

FIG. 3 depicts a co-encapsulation assay. Cells containing library clones are co-encapsulated with a substrate or labeled oligonucleotide. Encapsulation can occur in a variety of means, including GMDs, liposomes, and ghost cells. Cells are screened via high throughput screening on a fluorescence analyzer.

FIG. 4 depicts a side scatter versus forward scatter graph of FACS sorted gel-microdroplets (GMDs) containing a species of Streptomyces which forms unicells. Empty gel-microdroplets are distinguished from free cells and debris, also.

FIG. 5 is a depiction of a FACS/Biopanning method described herein and described in Example 3, below.

FIG. 6A shows an example of dimensions of a capillary array of the invention. FIG. 6B illustrates an array of capillary arrays.

FIG. 7 shows a top cross-sectional view of a capillary array.

FIG. 8 is a schematic depicting the excitation of and emission from a sample within the capillary lumen according to one aspect of the invention.

FIG. 9 is a schematic depicting the filtering of excitation and emission light to and from a sample within the capillary lumen according to an alternative aspect of the invention.

FIG. 10 illustrates an aspect of the invention in which a capillary array is wicked by contacting a sample containing cells, and humidified in a humidified incubator followed by imaging and recovery of cells in the capillary array.

FIG. 11 illustrates a method for incubating a sample in a capillary tube by an evaporative and capillary wicking cycle.

FIG. 12A shows a portion of a surface of a capillary array on which condensation has formed. FIG. 12B shows the portion of the surface of the capillary array, depicted in FIG. 12A, in which the surface is coated with a hydrophobic layer to inhibit condensation near an end of individual capillaries.

FIGS. 13A, 13B and 13C depict a method of retaining at least two components within a capillary.

FIG. 14A depicts capillary tubes containing paramagnetic beads and cells. FIG. 14B depicts the use of the paramagnetic beads to stir a sample in a capillary tube.

FIG. 15 depicts an excitation apparatus for a detection system according to an aspect of the invention.

FIG. 16 illustrates a system for screening samples using a capillary array according to an aspect of the invention.

FIG. 17A illustrates one example of a recovery technique useful for recovering a sample from a capillary array. In this depiction a needle is contacted with a capillary containing a sample to be obtained. A vacuum is created to evacuate the sample from the capillary tube and onto a filter. FIG. 17B illustrates one sample recovery method in which the recovery device has an outer diameter greater than the inner diameter of the capillary from which a sample is being recovered. FIG. 17C illustrates another sample recovery method in which the recovery device has an outer diameter approximately equal to or less than the inner diameter of the capillary. FIG. 17D shows the further processing of the sample once evacuated from the capillary.

FIG. 18 is a schematic showing high throughput enrichment of low copy gene targets.

FIG. 19 is a schematic of FACS-Biopanning using high throughput culturing. Polyketide synthase sequences from environmental samples are shown in the alignment.

FIG. 20 shows whole cell hybridization for biopanning.

FIG. 21 is a schematic showing co-encapsulation of a eukaryotic cell and a bacterial cell.

FIG. 22 illustrates a whole cell hybridization schematic for biopanning and FACS sorting.

FIG. 23 shows a schematic of T7 RNA Polymerase Expression system.

FIG. 24 is a schematic summarizing an exemplary protocol to determine the optimal growth medium for a broad diversity of organisms, as described in detail in Example 18, below.

FIG. 25 is an illustration of a light scattering signature of microcolonies as detected and separated by flow cytometry, as described in detail in Example 18, below.

FIGS. 26a, 26b and 26c are schematic drawings summarizing the characterization of clones (microcolonies) from organisms found and isolated by a method of the invention and analyzed by 16S rRNA gene sequence analysis, as described in detail in Example 18, below. FIG. 26d is an illustration of a picture of a culture designated as strain GMDJE10E6, as described in detail in Example 18, below.

FIG. 27 is a schematic drawing for a recombinant clone which has been characterized in Tier 1 as hydrolase and in Tier 2 as amide, which may then be tested in Tier 3 for various specificities.

FIGS. 28 and 29 are schematic drawings for a recombinant clone which has been characterized in Tier 1 as hydrolase and in Tier 2 as ester which may then be tested in Tier 3 for various specificities.

FIG. 30 is a schematic drawing for a recombinant clone which has been characterized in Tier 1 as hydrolase and in Tier 2 as acetal which may then be tested in Tier 3 for various specificities.

FIG. 31 is a schematic diagram of the procedure used to amplify trace amounts of environmental gDNA.

FIG. 32 is a table showing the results from using extracted gDNA as template, the template concentration lower limit was tested by serial dilutions. The MDA reaction gave no product yield below 10,000 cells (genomes). Using the Cut/Ligate method of template preparation, there was MDA reaction product from as little as 2 cells (genomes). Using the Reamplification method, it was shown that there was substantial product yield from straight, extracted gDNA from 1000 cells (genomes).

Like reference symbols in the various drawings indicate like elements.

### DETAILED DESCRIPTION

The methods of the present invention provide a novel approach to obtain and amplify trace amounts of whole genomic DNA derived from a plurality of organisms. In accordance with one aspect of the present invention, environmental samples that do not contain enough DNA for analysis by traditional methods are subject to multiple displacement amplification to enable the whole genomic DNA to be recovered and characterized as to physiological and metabolic potential.

This invention differs from multiple displacement amplification (MDA) and rolling circle amplification (RCA), as normally performed, in several aspects. Previously, MDA and RCA have been employed to expedite and simplify amplification of nucleic acid derived from single organisms. The DNA molecule is annealed with a primer molecule able to hybridize to it. The annealed mixture is incubated in a vessel containing four different deoxynucleoside triphosphates, a DNA polymerase, and one or more DNA synthesis terminating agents, which terminated DNA synthesis at a specific nucleotide base. The DNA products are then separated according to size. The DNA polymerase catalyzes primer extension and strand displacement in a processive strand displacement polymerization reaction. Use of a strand displacing DNA polymerase allows the reaction to proceed as long as desired in an isothermal reaction, while generating molecules of up to 60,000 nucleotides or larger.

In accordance with another aspect of the present invention, novel high throughput cultivation methods based on the combination of a single cell encapsulation procedure with flow cytometry that enables cells to grow with nutrients that are present at environmental concentrations are combined with the novel amplification methods to provide access to trace amounts of DNA within microcolonies for further analysis.

In a preferred embodiment, prior to amplification the gDNA is fragmented and then ligated to form self-ligated products. The DNA fragmentation can be achieved by enzymatic, chemical, photometric, mechanical (shearing) or any means that provides segments. Any enzymes used for fragmentation are then heat-inactivated. The DNA ends may be filled in using a DNA polymerase. The fragmented DNA is diluted to a degree sufficient to obtain substantially self-ligated products in the presence of ligase and ligase buffer. Any enzymes used for ligation are then heat-inactivated. The ligated products are added as template to the amplification reaction. At any step, the gDNA, fragmented DNA, or ligated DNA may be cleaned utilizing techniques known in the art.

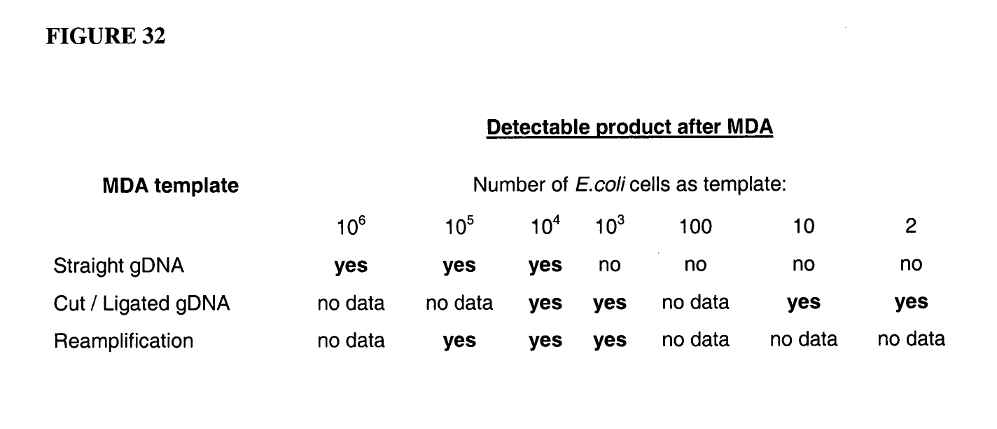

Using extracted gDNA as template, the template concentration lower limit was tested by serial dilutions. The MDA reaction gave no product yield below 10,000 cells (genomes). Using the Cut/Ligate method of template preparation, there was MDA reaction product from as little as 2 cells (genomes). (FIG. 32).

Amplification of nucleic acid from multiple organisms can be performed by mixing a set of random or partially random primers with a genomic sample from a mixed population of organisms to produce a primer-target sample mixture in a buffer solution. The mixture is incubated under conditions that promote hybridization between the primers and the genomic DNA in the primer-target sample mixture. A DNA polymerase is then added to produce a polymerase-target sample mixture, and incubated under conditions that promote replication of the genomic DNA. Strand displacement replication is preferably accomplished by using a strand displacing DNA polymerase or a DNA polymerase in combination with a compatible strand displacement factor.

In one embodiment of the present invention, the percent of DNA amplified comprises at least 10%, 20%, 30%, 40%, 50%, 60%, 70%, 80%, 90%, or 100% of the genome from the sample.

In another aspect of the invention, the amplification step may be repeated one or more times to achieve higher product yield. This is accomplished by using the reaction product as template for subsequent reactions. Some or all of the reaction is added together with additional reaction components and incubated for one or more hours. The addition of some or all of the reaction to additional reaction components, and incubation for one or more hours, may be done one or more times.

Using the Reamplification method, it was shown that there was substantial product yield from straight, extracted gDNA from 1000 cells (genomes). The considerable amount of product from 1000 cells shows that it should be possible to use the reamplification method on lower template concentrations. (FIG. 32).

Preferred strand displacing DNA polymerases are large fragment Bst DNA polymerase (Exo(−)Bst), exo(−)Bca DNA polymerase, the DNA polymerase of the bacteriophage Φ29 and Sequenase.

The present invention provides a method for rapid sorting and screening of libraries derived from trace amounts of DNA derived from a mixed population of organisms from, for example, an environmental sample or an uncultivated population of organisms. In one aspect, gene libraries are generated, clones are either exposed to a substrate or substrate(s) of interest, or hybridized to a fluorescence labeled probe having a sequence corresponding to a sequence of interest and positive clones are identified and isolated via fluorescence activated cell sorting. Cells can be viable or non-viable during the process or at the end of the process, as nucleic acids encoding a positive activity can be isolated and cloned utilizing techniques well known in the art.

This invention differs from fluorescence activated cell sorting, as normally performed, in several aspects. Previously, FACS machines have been employed in studies focused on the analyses of eukaryotic and prokaryotic cell lines and cell culture processes. FACS has also been utilized to monitor production of foreign proteins in both eukaryotes and prokaryotes to study, for example, differential gene expression. The detection and counting capabilities of the FACS system have been applied in these examples. However, FACS has never previously been employed in a discovery process to screen for and recover bioactivities in prokaryotes. In addition, non-optical methods have not been used to identify or discover novel bioactivities or biomolecules. Furthermore, the present invention does not require cells to survive, as do previously described technologies, since the desired nucleic acid (recombinant clones) can be obtained from alive or dead cells. For example, the cells only need to be viable long enough to contain, carry or synthesize a complementary nucleic acid sequence to be detected, and can thereafter be either viable or non-viable cells so long as the complementary sequence remains intact. The present invention also solves problems that would have been associated with detection and sorting of E. coli expressing recombinant enzymes, and recovering encoding nucleic acids. The invention includes within its aspects apparatus capable of detecting a molecule or marker that is indicative of a bioactivity or biomolecule of interest, including optical and non-optical apparatus.

In one aspect, the present invention includes within its aspects any apparatus capable of detecting fluorescent wavelengths associated with biological material, such apparatuses are defined herein as fluorescent analyzers (one example of which is a FACS apparatus).

In the methods of the invention, use of a culture-independent approach to directly clone genes encoding novel enzymes from, for example, an environmental sample containing trace amounts of DNA derived from a mixed population of organisms allows one to access untapped resources of biodiversity. In one aspect, the invention is based on the construction of “mixed population libraries” which represent the collective genomes of naturally occurring organisms archived in cloning vectors that can be propagated in suitable prokaryotic hosts. Because the cloned DNA is initially extracted directly from environmental samples, the libraries are not limited to the small fraction of prokaryotes that can be grown in pure culture. Additionally, a normalization of the DNA present in these samples could allow more equal representation of the DNA from all of the species present in the original sample. This can increase the efficiency of finding interesting genes from minor constituents of the sample which may be under-represented by several orders of magnitude compared to the dominant species.

Prior to the present invention, the evaluation of complex mixed population expression libraries was rate limiting. The present invention allows the rapid screening of complex mixed population libraries, containing, for example, genes from thousands of different organisms. The benefits of the present invention can be seen, for example, in screening a complex mixed population sample. Screening of a complex sample previously required one to use labor intensive methods to screen several million clones to cover the genomic biodiversity. The invention represents an extremely high-throughput screening method which allows one to assess this enormous number of clones. The method disclosed herein allows the screening anywhere from about 30 million to about 200 million clones per hour for a desired nucleic acid sequence or biological activity. This allows the thorough screening of mixed population libraries for clones expressing novel biomolecules.

The invention provides methods and compositions whereby one can screen, sort or identify a polynucleotide sequence, polypeptide, or molecule of interest from a mixed population of organisms (e.g., organisms present in a mixed population sample) based on polynucleotide sequences present in the sample. Thus, the invention provides methods and compositions useful in screening organisms for a desired biological activity or biological sequence and to assist in obtaining sequences of interest that can further be used in directed evolution, molecular biology, biotechnology and industrial applications. By screening and identifying the nucleic acid sequences present in the sample, the invention increases the repertoire of available sequences that can be used for the development of diagnostics, therapeutics or molecules for industrial applications. Accordingly, the methods of the invention can identify novel nucleic acid sequences encoding proteins or polypeptides having a desired biological activity.

In one aspect, the invention provides a method for high throughput culturing of organisms. In another aspect, the organisms are a mixed population of organisms. In another aspect, organisms comprise a minute amount of cells. In another aspect, trace amounts of DNA are derived from the mixed population of organisms. In another aspect, the organisms include host cells of a library containing nucleic acids. For example, such libraries include nucleic acid obtained from various isolates of organisms, which are then pooled; nucleic acid obtained from isolate libraries, which are then pooled; or nucleic acids derived directly from a mixed population of organisms. Generally, a sample containing the organisms is mixed with a composition that can form a microenvironment, as described herein, e.g., a gel microdroplet or a liposome. In one aspect, a mixed population of microorganisms is mixed with the encapsulation material in such a way that preferably fewer than 5 microorganisms are encapsulated. Preferably, only one microorganism is encapsulated in each microenvironment system.

Once encapsulated, the cells are cultured in a manner which allows growth of the organisms, e.g., host cells of a library. For example, Example 9 provides growth of the encapsulated organisms in a chromatography column which allows a flow of growth medium providing nutrients for growth and for removal of waste products from cells. Over a period of time (20 minutes to several weeks or months), a clonal population (i.e., microcolony) of the preferably one organism grows within the microenvironment.

After a desired period of time, microenvironments, e.g., gel microdroplets, can be sorted to eliminate “empty” microenvironments and to sort for the occupied microenvironments. The nucleic acid from organisms in the sorted microenvironments can be studied directly, for example, by treating with a PCR mixture and amplified immediately after sorting. In one Example described herein, 16S rRNA genes from individual cells were studied and organisms assessed for phylogenetic diversity from the samples. If only trace amounts of DNA are derived from the microcolony, the nucleic acid is amplified by multiple displacement amplification.

In another aspect, the high throughput culturing methods of the invention allow culturing of organisms and enrichment of low copy gene targets. For example, a library of nucleic acid obtained from various isolates of organisms, which are then pooled; nucleic acid obtained from isolate libraries, which are then pooled; or nucleic acids derived directly from a mixed population of organisms, for example, are encapsulated, e.g., in a gel microdroplet or other microenvironment, and grown under conditions which allow clonal expansion of each organism in the microenvironment. In one aspect, the cells of the microcolony are lysed and treated with proteinases to yield nucleic acid (see Figures) (e.g., the microcolonies are de-proteinized by incubating gel microdroplets in lysis solution containing proteinase K at 37 degrees C. for 30 minutes). In order to denature and neutralize nucleic acid entrapped in the microenvironments, they are denatured with alkaline denaturing solution (0.5M NaOH) and neutralized (e.g., with Tris pH8). In one particular example, nucleic acid entrapped in the microenvironment is hybridized with Digoxiginin (DIG)-labeled oligonucleotides (30-50 nt) in Dig Easy Hyb (available from Roche) overnight at 37 degrees C., followed by washing with 0.3×SSC and 0.1×SSC at 38-50 degrees C. to achieve desired stringency. One of skill in the art will appreciate that this is merely an example and not meant to limit the invention in any way. For example, other labels commonly used in the art, e.g., fluorescent labels such as GFP or chemiluminescent labels, can be utilized in the invention methods.

The nucleic acid is hybridized with a probe which is preferably labeled. A signal can be amplified with a secondary label (e.g., fluorescent) and the nucleic acid sorted for fluorescent microenvironments, e.g., gel microdroplets. Nucleic acid that is fluorescent can be isolated and further studied or cloned into a host cell for further manipulation. In one particular example, signals are amplified with Tyramide Signal Amplification™ (TSA) kit from Molecular Probe. TSA is an enzyme-mediated signal amplification method that utilizes horseradish peroxidase (HRP) to depose fluorogenic tyramide molecules and generate high-density labeling of a target nucleic acid sequence in situ. The signal amplification is conferred by the turnover of multiple tyramide substrates per HRP molecule, and increases in signal strength of over 1,000-fold have been reported. The procedure involves incubating GMDs with anti-DIG conjugated horseradish peroxidase (anti-DIG-HRP) (Roche, Ind.) for 3 hours at room temperature. Then the tyramide substrate solution will be added and incubated for 30 minutes at room temperature (RT).

In one aspect, this high throughput culturing method followed by sorting (e.g., FACS) screening (e.g., biopanning), allows for identification of gene targets. It may be desirable to screen for nucleic acids encoding virtually any protein or any bioactivity and to compare such nucleic acids among various species of organisms in a sample (e.g., study polyketide sequences from a mixed population). In another aspect, nucleic acid derived from high throughput culturing of organisms can be obtained for further study or for generation of a library. Such nucleic acid can be pooled and a library created, or alternatively, individual libraries from clonal populations (i.e., microcolonies) of organisms can be generated and then nucleic acid pooled from those libraries to generate a more complex library. The libraries generated as described herein can be utilized for the discovery of biomolecules (e.g., nucleic acid or bioactivities) or for evolving nucleic acid molecules identified by the high throughput culturing methods described in the present invention.

Such evolution methods are known in the art or described herein, such as, shuffling, cassette mutagenesis, recursive ensemble mutagenesis, sexual PCR, directed evolution, exonuclease-mediated reassembly, codon site-saturation mutagenesis, amino acid site-saturation mutagenesis, gene site saturation mutagenesis, introduction of mutations by non-stochastic polynucleotide reassembly methods, synthetic ligation polynucleotide reassembly, gene reassembly, oligonucleotide-directed saturation mutagenesis, in vivo reassortment of polynucleotide sequences having partial homology, naturally occurring recombination processes which reduce sequence complexity, and any combination thereof.

Flow cytometry has been used in cloning and selection of variants from existing cell clones. This selection, however, has required stains that diffuse through cells passively, rapidly and irreversibly, with no toxic effects or other influences on metabolic or physiological processes. Since, typically, flow sorting has been used to study animal cell culture performance, physiological state of cells, and the cell cycle, one goal of cell sorting has been to keep the cells viable during and after sorting.

There currently are no reports in the literature of screening and discovery of polynucleotide sequence in libraries by cell sorting based on fluorescence (e.g. fluorescent activated cell sorting), or non-optical markers (e.g., magnetic fields and the like). Furthermore there are no reports of recovering DNA encoding bioactivities screened by FACS or non-optical techniques and additionally screening for a bioactivity of interest. The present invention provides these methods to allow the extremely rapid screening of viable or non-viable cells to recover desirable activities and the nucleic acid encoding those activities.

Different types of encapsulation (e.g., gel microdroplet) strategies and compounds or polymers can be used with the present invention. For instance, high temperature agaroses can be employed for making microdroplets stable at high temperatures, allowing stable encapsulation of cells subsequent to heat-kill steps utilized to remove all background activities when screening for thermostable bioactivities. Encapsulation can be in beads, high temperature agaroses, gel microdroplets, cells, such as ghost red blood cells or macrophages, liposomes, or any other means of encapsulating and localizing molecules. For example, methods of preparing liposomes have been described (i.e., U.S. Pat. Nos. 5,653,996, 5,393,530 and 5,651,981), as well as the use of liposomes to encapsulate a variety of molecules U.S. Pat. Nos. 5,595,756, 5,605,703, 5,627,159, 5,652,225, 5,567,433, 4,235,871, 5,227,170). Entrapment of proteins, viruses, bacteria and DNA in erythrocytes during endocytosis has been described, as well (Journal of Applied Biochemistry 4, 418-435 (1982)). Erythrocytes employed as carriers in vitro or in vivo for substances entrapped during hypo-osmotic lysis or dielectric breakdown of the membrane have also been described (reviewed in Ihler, G. M. (1983) J. Pharm. Ther). These techniques are useful in the present invention to encapsulate samples for screening.

“Microenvironment”, as used herein, is any molecular structure which provides an appropriate environment for facilitating the interactions necessary for the method of the invention. An environment suitable for facilitating molecular interactions include, for example, gel microdroplets, agarose noodles, ghost cells, macrophages or liposomes.

Liposomes can be prepared from a variety of lipids including phospholipids, glycolipids, steroids, long-chain alkyl esters; e.g., alkyl phosphates, fatty acid esters; e.g., lecithin, fatty amines and the like. A mixture of fatty material may be employed such a combination of neutral steroid, a charge amphiphile and a phospholipid. Illustrative examples of phospholipids include lecithin, sphingomyelin and dipalmitoylphosphatidylcholine. Representative steroids include cholesterol, cholestanol and lanosterol. Representative charged amphiphilic compounds generally contain from 12-30 carbon atoms. Mono- or dialkyl phosphate esters, or alkyl amines; e.g., dicetyl phosphate, stearyl amine, hexadecyl amine, dilauryl phosphate, and the like.

The invention methods include a system and method for holding and screening samples. According to one aspect of the invention, a sample screening apparatus includes a plurality of capillaries formed into an array of adjacent capillaries, wherein each capillary comprises at least one wall defining a lumen for retaining a sample. The apparatus further includes interstitial material disposed between adjacent capillaries in the array, and one or more reference indicia formed within of the interstitial material. (see co-pending U.S. patent applications Ser. Nos. 09/687,219 and 09/894,956).

According to another aspect of the invention, a capillary for screening a sample, wherein the capillary is adapted for being bound in an array of capillaries, includes a first wall defining a lumen for retaining the sample, and a second wall formed of a filtering material, for filtering excitation energy provided to the lumen to excite the sample.

In another aspect of the invention, a method for incubating a bioactivity or biomolecule of interest includes the steps of introducing a first component into at least a portion of a capillary of a capillary array, wherein each capillary of the capillary array comprises at least one wall defining a lumen for retaining the first component, and introducing an air bubble into the capillary behind the first component. The method further includes the step of introducing a second component into the capillary, wherein the second component is separated from the first component by the air bubble.

In one aspect of the invention, a method of incubating a sample of interest includes introducing a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method further includes removing the first liquid from the capillary tube, wherein the bound detectable particle is maintained within the capillary, and introducing a second liquid into the capillary tube.

Another aspect of the invention includes a recovery apparatus for a sample screening system, wherein the system includes a plurality of capillaries formed into an array. The recovery apparatus includes a recovery tool adapted to contact at least one capillary of the capillary array and recover a sample from the at least one capillary. The recovery apparatus further includes an ejector, connected with the recovery tool, for ejecting the recovered sample from the recovery tool.

Definitions

Unless defined otherwise, all technical and scientific terms used herein have the same meaning as commonly understood to one of ordinary skill in the art to which the invention belongs. Although any methods, devices and materials similar or equivalent to those described herein can be used in the practice or testing of the invention, the methods, devices and materials are now described.

As used herein and in the appended claims, the singular forms “a,” “and,” and “the” include plural referents unless the context clearly dictates otherwise. Thus, for example, reference to “a clone” includes a plurality of clones and reference to “the nucleic acid sequence” generally includes reference to one or more nucleic acid sequences and equivalents thereof known to those skilled in the art, and so forth.

An “amino acid” is a molecule having the structure wherein a central carbon atom (the β-carbon atom) is linked to a hydrogen atom, a carboxylic acid group (the carbon atom of which is referred to herein as a “carboxyl carbon atom”), an amino group (the nitrogen atom of which is referred to herein as an “amino nitrogen atom”), and a side chain group, R. When incorporated into a peptide, polypeptide, or protein, an amino acid loses one or more atoms of its amino acid carboxylic groups in the dehydration reaction that links one amino acid to another. As a result, when incorporated into a protein, an amino acid is referred to as an “amino acid residue.”

“Protein” or “polypeptide” refers to any polymer of two or more individual amino acids (whether or not naturally occurring) linked via a peptide bond, and occurs when the carboxyl carbon atom of the carboxylic acid group bonded to the β-carbon of one amino acid (or amino acid residue) becomes covalently bound to the amino nitrogen atom of amino group bonded to the β-carbon of an adjacent amino acid. The term “protein” is understood to include the terms “polypeptide” and “peptide” (which, at times may be used interchangeably herein) within its meaning. In addition, proteins comprising multiple polypeptide subunits (e.g., DNA polymerase III, RNA polymerase II) or other components (for example, an RNA molecule, as occurs in telomerase) will also be understood to be included within the meaning of “protein” as used herein. Similarly, fragments of proteins and polypeptides are also within the scope of the invention and may be referred to herein as “proteins.”

A particular amino acid sequence of a given protein (i.e., the polypeptide's “primary structure,” when written from the amino-terminus to carboxy-terminus) is determined by the nucleotide sequence of the coding portion of a mRNA, which is in turn specified by genetic information, typically genomic DNA (including organelle DNA, e.g., mitochondrial or chloroplast DNA). Thus, determining the sequence of a gene assists in predicting the primary sequence of a corresponding polypeptide and more particular the role or activity of the polypeptide or proteins encoded by that gene or polynucleotide sequence.

The term “isolated” means altered “by the hand of man” from its natural state; i.e., if it occurs in nature, it has been changed or removed from its original environment, or both. For example, a naturally occurring polynucleotide or a polypeptide naturally present in a living animal, a biological sample or an environmental sample in its natural state is not “isolated”, but the same polynucleotide or polypeptide separated from the coexisting materials of its natural state is “isolated”, as the term is employed herein. Such polynucleotides, when introduced into host cells in culture or in whole organisms, still would be isolated, as the term is used herein, because they would not be in their naturally occurring form or environment. Similarly, the polynucleotides and polypeptides may occur in a composition, such as a media formulation (solutions for introduction of polynucleotides or polypeptides, for example, into cells or compositions or solutions for chemical or enzymatic reactions).

“Polynucleotide” or “nucleic acid sequence” refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The tern therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxy-ribonucleotides, or modified forms of either nucleotide. A polynucleotides as used herein refers to, among others, single-and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term polynucleotide encompasses genomic DNA or RNA (depending upon the organism, i.e., RNA genome of viruses), as well as mRNA encoded by the genomic DNA, and cDNA.

The term “trace” means an extremely small but detectable quantity. When used in conjunction with DNA (e.g., “trace amount of DNA”), it is meant to describe DNA in quantities not suitable for analysis by traditional methods such as sequencing and library construction. When used in conjunction with cells (e.g., “trace amount of cells”), it is meant to describe approximately 1-1000 cells, which may also be called a “microcolony” if the cells were cultured from a single cell. Trace amounts of DNA or cells may also describe the amount of at least one species in the environmental sample or the environmental sample as a whole.

In one embodiment, the methods of the present inventiona are suitable for use in environmental samples where 1, 2, 3, 4, less than 5, less than 10, less than 100, less than 1000 cells of any one species is present in the sample.

In another embodiment, the methods of the present invention may be used when there is 0.1-200 million femtograms of any one organism present in an environmental sample. One skilled in the art would understand that the complexity of an organism's genome as compared to E. coli, for example, would require more DNA to obtain a full representation of the organism's genome.

The term “fragment,” “fragments,” and the grammatical equivalents thereof as used herein means a segment of sufficient size to allow ligation of a nucleic acid sequence into a circle by any method know in the art.

By rapidly screening for polynucleotides encoding polypeptides of interest, the invention provides not only a source of materials for the development of biologics, therapeutics, and enzymes for industrial applications, but also provides a new materials for further processing by, for example, directed evolution and mutagenesis to develop molecules or polypeptides modified for particular activity or conditions.

The invention is used to obtain and identify polynucleotides and related sequence specific information from, for example, infectious microorganisms present in the environment such as, for example, in the gut of various macroorganisms.

In another aspect, the methods and compositions of the invention provide for the identification of lead drug compounds present in an environmental sample. The methods of the invention provide the ability to mine the environment for novel drugs or identify related drugs contained in different microorganisms. There are several common sources of lead compounds (drug candidates), including natural product collections, synthetic chemical collections, and synthetic combinatorial chemical libraries, such as nucleotides, peptides, or other polymeric molecules that have been identified or developed as a result of environmental mining. Each of these sources has advantages and disadvantages. The success of programs to screen these candidates depends largely on the number of compounds entering the programs, and pharmaceutical companies have to date screened hundred of thousands of synthetic and natural compounds in search of lead compounds. Unfortunately, the ratio of novel to previously-discovered compounds has diminished with time. The discovery rate of novel lead compounds has not kept pace with demand despite the best efforts of pharmaceutical companies. There exists a strong need for accessing new sources of potential drug candidates. Accordingly, the invention provides a rapid and efficient method to identify and characterize environmental samples that may contain novel drug compounds.

The invention provides methods of identifying a nucleic acid sequence encoding a polypeptide having either known or unknown function. For example, much of the diversity in microbial genomes results from the rearrangement of gene clusters in the genome of microorganisms. These gene clusters can be present across species or phylogenetically related with other organisms.

For example, bacteria and many eukaryotes have a coordinated mechanism for regulating genes whose products are involved in related processes. The genes are clustered, in structures referred to as “gene clusters,” on a single chromosome and are transcribed together under the control of a single regulatory sequence, including a single promoter which initiates transcription of the entire cluster. The gene cluster, the promoter, and additional sequences that function in regulation altogether are referred to as an “operon” and can include up to 20 or more genes, usually from 2 to 6 genes. Thus, a gene cluster is a group of adjacent genes that are either identical or related, usually as to their function. Gene clusters are generally 15 kb to greater than 120 kb in length.

Some gene families consist of identical members. Clustering is a prerequisite for maintaining identity between genes, although clustered genes are not necessarily identical. Gene clusters range from extremes where a duplication is generated to adjacent related genes to cases where hundreds of identical genes lie in a tandem array. Sometimes no significance is discernable in a repetition of a particular gene. A principal example of this is the expressed duplicate insulin genes in some species, whereas a single insulin gene is adequate in other mammalian species.

Further, gene clusters undergo continual reorganization and, thus, the ability to create heterogeneous libraries of gene clusters from, for example, bacterial or other prokaryote sources is valuable in determining sources of novel proteins, particularly including enzymes such as, for example, the polyketide synthases that are responsible for the synthesis of polyketides having a vast array of useful activities. Other types of proteins that are the product(s) of gene clusters are also contemplated, including, for example, antibiotics, antivirals, antitumor agents and regulatory proteins, such as insulin.

As an example, polyketide syntheses enzymes fall in a gene cluster. Polyketides are molecules which are an extremely rich source of bioactivities, including antibiotics (such as tetracyclines and erythromycin), anti-cancer agents (daunomycin), immunosuppressants (FK506 and rapamycin), and veterinary products (monensin). Many polyketides (produced by polyketide syntheses) are valuable as therapeutic agents. Polyketide synthases are multifunctional enzymes that catalyze the biosynthesis of a huge variety of carbon chains differing in length and patterns of functionality and cyclization. Polyketide synthase genes fall into gene clusters and at least one type (designated type I) of polyketide synthases have large size genes and enzymes, complicating genetic manipulation and in vitro studies of these genes/proteins.

The ability to select and combine desired components from a library of polyketides and postpolyketide biosynthesis genes for generation of novel polyketides for study is appealing. The method(s) of the present invention make it possible to, and facilitate the cloning of, novel polyketide synthases, since one can generate gene banks with clones containing large inserts (especially when using the f-factor based vectors), which facilitates cloning of gene clusters.

Other biosynthetic genes include NRPS, glycosyl transferases and p450s. For example, a gene cluster can be ligated into a vector containing an expression regulatory sequences which can control and regulate the production of a detectable protein or protein-related array activity from the ligated gene clusters. Use of vectors which have an exceptionally large capacity for exogenous nucleic acid introduction are particularly appropriate for use with such gene clusters and are described by way of example herein to include artificial chromosome vectors, cosmids, and the f-factor (or fertility factor) of E. coli. For example, the f-factor of E. coli is a plasmid which affects high-frequency transfer of itself during conjugation and is ideal to achieve and stably propagate large nucleic acid fragments, such as gene clusters from samples of mixed populations of organisms.

The trace amounts of DNA isolated or derived from these microorganisms can preferably be amplified then inserted into a vector prior to probing for selected DNA. Such vectors are preferably those containing expression regulatory sequences, including promoters, enhancers and the like. Such polynucleotides can be part of a vector and/or a composition and still be isolated, in that such vector or composition is not part of its natural environment. Particularly preferred phages or plasmids, and methods for introduction and packaging into them, are described in detail in the protocol set forth herein.

The invention provides novel systems to clone and screen mixed populations of organisms present, for example, in environmental samples, for polynucleotides of interest, enzymatic activities and bioactivities of interest in vitro. The method(s) of the invention allow the cloning and discovery of novel bioactive molecules in vitro, and in particular novel bioactive molecules derived from uncultivated or cultivated samples. Large size gene clusters, genes and gene fragments can be cloned, sequenced and screened using the method(s) of the invention. Unlike previous strategies, the method(s) of the invention allow one to clone, screen and identify polynucleotides and the polypeptides encoded by these polynucleotides in vitro from a wide range of mixed population samples.

The invention allows one to screen for and identify polynucleotide sequences from complex mixed population samples. DNA libraries obtained from trace amounts of DNA from these samples can be created from cell free samples, so long as the sample contains nucleic acid sequences, or from samples containing cellular organisms or viral particles. The organisms from which the libraries may be prepared include prokaryotic microorganisms, such as Eubacteria and Archaebacteria, lower eukaryotic microorganisms such as fungi, algae and protozoa, as well as plants, plant spores and pollen. The organisms may be cultured organisms or uncultured organisms obtained from mixed population environmental samples, including extremophiles, such as thermophiles, hyperthermophiles, psychrophiles, psychrotrophs, halophiles, alkalophiles, and acidophiles.

Sources of nucleic acids used to construct a DNA library can be obtained from mixed population samples, such as, but not limited to, microbial samples obtained from Arctic and Antarctic ice, water or permafrost sources, materials of volcanic origin, materials from soil or plant sources in tropical areas, droppings from various organisms including mammals, invertebrates, dead and decaying matter, contaminated soil samples such as from radioactive waste sites and toxic spill sites, etc. Thus, for example, nucleic acids may be recovered from either a cultured or non-cultured organism and used to produce an appropriate DNA library (e.g., a recombinant expression library) for subsequent determination of the identity of the particular polynucleotide sequence or screening for bioactivity

The following outlines a general procedure for producing libraries from both culturable and non-culturable organisms as well as mixed population of organisms, which libraries can be probed, sequenced or screened to select therefrom nucleic acid sequences having an identified, desired or predicted biological activity (e.g., an enzymatic activity or a small molecule).

As used herein a mixed population sample is any sample containing organisms or polynucleotides or a combination thereof, which can be obtained from any number of sources (as described above), including, for example, insect feces, soil, water, etc. Any source of nucleic acids in purified or non-purified form can be utilized as starting material. Thus, the nucleic acids may be obtained from any source which is contaminated by an organism or from any sample containing cells. The mixed population sample can be an extract from any bodily sample such as blood, urine, spinal fluid, tissue, vaginal swab, stool, amniotic fluid or buccal mouthwash from any mammalian organism. For non-mammalian (e.g., invertebrates) organisms the sample can be a tissue sample, salivary sample, fecal material or material in the digestive tract of the organism. An environmental sample also includes samples obtained from extreme environments including, for example, hot sulfur pools, volcanic vents, and frozen tundra. In addition, the sample can come from a variety of sources. For example, in horticulture and agricultural testing the sample can be a plant, fertilizer, soil, liquid or other horticultural or agricultural product; in food testing the sample can be fresh food or processed food (for example infant formula, seafood, fresh produce and packaged food); and in environmental testing the sample can be liquid, soil, sewage treatment, sludge and any other sample in the environment which is considered or suspected of containing an organism or polynucleotides.

When the sample is a mixture of material (e.g., a mixed population of organisms), for example, blood, soil and sludge, it can be treated with an appropriate reagent which is effective to open the cells and expose or separate the strands of nucleic acids. Mixed populations can comprise pools of cultured organisms or samples. For example, samples of organisms can be cultured prior to analysis in order to purify a particular population and thus obtaining a purer sample. Organisms, such as actinomycetes or myxobacteria, known to produce bioactivities of interest can be enriched for, via culturing. Culturing of organisms in the sample can include culturing the organisms in microdroplets and separating the cultured microdroplets with a cell sorter into individual wells of a multi-well tissue culture plate from which further processing may be performed.

The sample can comprise nucleic acids from, for example, a diverse and mixed population of organisms (e.g., microorganisms present in the gut of an insect). When present in trace amounts, the DNA is subject to multiple displacement amplification. Nucleic acids are then isolated from the sample using any number of methods for DNA and RNA isolation. Such nucleic acid isolation methods are commonly performed in the art. Where the nucleic acid is RNA, the RNA can be reversed transcribed to DNA using primers known in the art. Where the DNA is genomic DNA, the DNA can be sheared using, for example, a 25 gauge needle.

The nucleic acids can be cloned into a vector. Cloning techniques are known in the art or can be developed by one skilled in the art, without undue experimentation. Vectors used in the present invention include: plasmids, phages, cosmids, phagemids, viruses (e.g., retroviruses, parainfluenzavirus, herpesviruses, reoviruses, paramyxoviruses, and the like), artificial chromosomes, or selected portions thereof (e.g., coat protein, spike glycoprotein, capsid protein). For example, cosmids and phagemids are typically used where the specific nucleic acid sequence to be analyzed or modified is large because these vectors are able to stably propagate large polynucleotides.

The vector containing the cloned DNA sequence can then be amplified by plating (i.e., clonal amplification) or transfecting a suitable host cell with the vector (e.g., a phage on an E. coli host). Alternatively (or subsequently to amplification), the cloned DNA sequence is used to prepare a library for screening by transforming a suitable organism. Hosts, known in the art are transformed by artificial introduction of the vectors containing the target nucleic acid by inoculation under conditions conducive for such transformation. One could transform with double stranded circular or linear nucleic acid or there may also be instances where one would transform with single stranded circular or linear nucleic acid sequences. By transform or transformation is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell. A transformed cell or host cell generally refers to a cell (e.g., prokaryotic or eukaryotic) into which (or into an ancestor of which) has been introduced, by means of recombinant DNA techniques, a DNA molecule not normally present in the host organism.

A particularly preferred type of vector for use in the invention contains an f-factor origin replication. The f-factor (or fertility factor) in E. coli is a plasmid which effects high frequency transfer of itself during conjugation and less frequent transfer of the bacterial chromosome itself. In a particular aspect cloning vectors referred to as “fosmids” or bacterial artificial chromosome (BAC) vectors are used. These are derived from E. coli f-factor which is able to stably integrate large segments of DNA. When integrated with DNA from a mixed uncultured mixed population sample, this makes it possible to achieve large genomic fragments in the form of a stable “mixed population nucleic acid library.”

The nucleic acids derived from a mixed population or sample may be inserted into the vector by a variety of procedures. In general, the nucleic acid sequence is inserted into an appropriate restriction endonuclease site(s) by procedures known in the art. Such procedures and others are deemed to be within the scope of those skilled in the art. A typical cloning scenario may have the DNA “blunted” with an appropriate nuclease (e.g., Mung Bean Nuclease), methylated with, for example, EcoR I Methylase and ligated to EcoR I linkers. The linkers are then digested with an EcoR I Restriction Endonuclease and the DNA size fractionated (e.g., using a sucrose gradient). The resulting size fractionated DNA is then ligated into a suitable vector for sequencing, screening or expression (e.g., a lambda vector and packaged using an in vitro lambda packaging extract).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation. Transformation of Pseudomonas fluorescens and yeast host cells can be achieved by electroporation, using techniques described herein.

When the host is a eukaryote, methods of transfection or transformation with DNA include conjugation, calcium phosphate co-precipitates, conventional mechanical procedures such as microinjection, electroporation, insertion of a plasmid encased in liposomes, or virus vectors, as well as others known in the art, may be used. Eukaryotic cells can also be cotransfected with a second foreign DNA molecule encoding a selectable marker, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). The eukaryotic cell may be a yeast cell (e.g., Saccharomyces cerevisiae), an insect cell (e.g., Drosophila sp.) or may be a mammalian cell, including a human cell.

Eukaryotic systems, and mammalian expression systems, allow for post-translational modifications of expressed mammalian proteins to occur. Eukaryotic cells which possess the cellular machinery for processing of the primary transcript, glycosylation, phosphorylation, and, advantageously secretion of the gene product should be used. Such host cell lines may include, but are not limited to, CHO, VERO, BHK, HeLa, COS, MDCK, Jurkat, HEK-293, and W138.

After the gene libraries have been generated one can perform “biopanning” of the libraries prior to expression screening. The “biopanning” procedure refers to a process for identifying clones having a specified biological activity by screening for sequence homology in the library of clones, using at least one probe DNA comprising at least a portion of a DNA sequence encoding a polypeptide having the specified biological activity; and detecting interactions with the probe DNA to a substantially complementary sequence in a clone. Clones (either viable or non-viable) are then separated by an analyzer (e.g., a FACS apparatus or an apparatus that detects non-optical markers).

The probe DNA used to probe for the target DNA of interest contained in clones prepared from polynucleotides in a mixed population of organisms can be a full-length coding region sequence or a partial coding region sequence of DNA for a known bioactivity. The sequence of the probe can be generated by synthetic or recombinant means and can be based upon computer based sequencing programs or biological sequences present in a clone. The DNA library can be probed using mixtures of probes comprising at least a portion of the DNA sequence encoding a known bioactivity having a desired activity. These probes or probe libraries are preferably single-stranded. The probes that are particularly suitable are those derived from DNA encoding bioactivities having an activity similar or identical to the specified bioactivity which is to be screened.

In another aspect, a nucleic acid library from a mixed population of organisms is screened for a sequence of interest by transfecting a host cell containing the library with at least one labeled nucleic acid sequence which is all or a portion of a DNA sequence encoding a bioactivity having a desirable activity and separating the library clones containing the desirable sequence by optical- or non-optical-based analysis.

In another aspect, in vivo biopanning may be performed utilizing a FACS-based machine. Complex gene libraries are constructed with vectors which contain elements which stabilize transcribed RNA. For example, the inclusion of sequences which result in secondary structures such as hairpins which are designed to flank the transcribed regions of the RNA would serve to enhance their stability, thus increasing their half life within the cell. The probe molecules used in the biopanning process consist of oligonucleotides labeled with reporter molecules that only fluoresce upon binding of the probe to a target molecule. Various dyes or stains well known in the art, for example those described in “Practical Flow Cytometry”, 1995 Wiley-Liss, Inc., Howard M. Shapiro, M.D., can be used to intercalate or associate with nucleic acid in order to “label” the oligonucleotides. These probes are introduced into the recombinant cells of the library using one of several transformation methods. The probe molecules interact or hybridize to the transcribed target mRNA or DNA resulting in DNA/RNA heteroduplex molecules or DNA/DNA duplex molecules. Binding of the probe to a target will yield a fluorescent signal which is detected and sorted by the FACS machine during the screening process.

The probe DNA can be at least about 10 bases, or, at least 15 bases. Other size ranges for probe DNA are at least about 15 bases to about 100 bases, at least about 100 bases to about 500 bases, at least about 500 bases to about 1,000 bases, at least about 1,000 bases to about 5,000 bases and at least about 5,000 bases to about 10,000 bases. In one aspect, an entire coding region of one part of a pathway may be employed as a probe. Where the probe is hybridized to the target DNA in an in vitro system, conditions for the hybridization in which target DNA is selectively isolated by the use of at least one DNA probe will be designed to provide a hybridization stringency of at least about 50% sequence identity, more particularly a stringency providing for a sequence identity of at least about 70%. Hybridization techniques for probing a microbial DNA library to isolate target DNA of potential interest are well known in the art and any of those which are described in the literature are suitable for use herein. Prior to fluorescence sorting the clones may be viable or non-viable. For example, in one aspect, the cells are fixed with paraformaldehyde prior to sorting.

Once viable or non-viable clones containing a sequence substantially complementary to the probe DNA are separated by a fluorescence analyzer, polynucleotides present in the separated clones may be further manipulated. In some instances, it may be desirable to perform an amplification of the target DNA that has been isolated. In this aspect, the target DNA is separated from the probe DNA after isolation. In one aspect, the clone can be grown to expand the clonal population. Alternatively, the host cell is lysed and the target DNA amplified. It is then amplified before being used to transform a new host (e.g., subcloning). Long PCR (Barnes, W M, Proc. Natl. Acad. Sci, USA, Mar. 15, 1994) can be used to amplify large DNA fragments (e.g., 35 kb). Numerous amplification methodologies are now well known in the art.

Where the target DNA is identified in vitro, the selected DNA is then used for preparing a library for further processing and screening by transforming a suitable organism. Hosts can be transformed by artificial introduction of a vector containing a target DNA by inoculation under conditions conducive for such transformation.

The resultant libraries (enriched for a polynucleotide of interest) can then be screened for clones which display an activity of interest. Clones can be shuttled in alternative hosts for expression of active compounds, or screened using methods described herein.

Having prepared a multiplicity of clones from DNA selectively isolated via hybridization technologies described herein, such clones are screened for a specific activity to identify clones having a specified characteristic.

The screening for activity may be effected on individual expression clones or may be initially effected on a mixture of expression clones to ascertain whether or not the mixture has one or more specified activities. If the mixture has a specified activity, then the individual clones may be rescreened for such activity or for a more specific activity.

Prior to, subsequent to or as an alternative to the in vivo biopanning described above is an encapsulation technique such as GMDs, which may be employed to localize at least one clone in one location for growth or screening by a fluorescent analyzer (e.g. FACS). The separated at least one clone contained in the GMD may then be cultured to expand the number of clones or screened on a FACS machine to identify clones containing a sequence of interest as described above, which can then be broken out into individual clones to be screened again on a FACS machine to identify positive individual clones. Screening in this manner using a FACS machine is described in patent application Ser. No. 08/876,276, filed Jun. 16, 1997. Thus, for example, if a clone has a desirable activity, then the individual clones may be recovered and rescreened utilizing a FACS machine to determine which of such clones has the specified desirable activity.

Further, it is possible to combine some or all of the above aspects such that a normalization step is performed prior to generation of the expression library, the expression library is then generated, the expression library so generated is then biopanned, and the biopanned expression library is then screened using a high throughput cell sorting and screening instrument. Thus there are a variety of options, including: (i) generating the library and then screening it; (ii) normalize the target DNA, generate the expression library and screen it; (iii) normalize, generate the library, biopan and screen; or (iv) generate, biopan and screen the library.

The library may, for example, be screened for a specified enzyme activity. For example, the enzyme activity screened for may be one or more of the six IUB classes; oxidoreductases, transferases, hydrolases, lyases, isomerases and ligases. The recombinant enzymes which are determined to be positive for one or more of the IUB classes may then be rescreened for a more specific enzyme activity.

Alternatively, the library may be screened for a more specialized protein, e.g. enzyme, activity. For example, instead of generically screening for hydrolase activity, the library may be screened for a more specialized activity, i.e. the type of bond on which the hydrolase acts. Thus, for example, the library may be screened to ascertain those hydrolases which act on one or more specified chemical functionalities, such as: (a) amide (peptide bonds), i.e. proteases; (b) ester bonds, i.e. esterases and lipases; (c) acetals, i.e., glycosidases etc.

As described with respect to one of the above aspects, the invention provides a process for activity screening of clones containing trace amounts of DNA derived from a mixed population of organisms or more than one organism.

Biopanning polynucleotides from a mixed population of organisms by separating the clones or polynucleotides positive for sequence of interest with a fluorescent analyzer that detects fluorescence, to select polynucleotides or clones containing polynucleotides positive for a sequence of interest, and screening the selected clones or polynucleotides for specified bioactivity. In one aspect, the polynucleotides are contained in clones having been prepared by recovering trace amounts of DNA of a plurality of microorganisms, which DNA is selected by hybridization to at least one DNA sequence which is all or a portion of a DNA sequence encoding a bioactivity having a desirable activity.

In another aspect, a DNA library derived from a plurality of microorganisms is subjected to a selection procedure to select therefrom DNA which hybridizes to one or more probe DNA sequences which is all or a portion of a DNA sequence encoding an activity having a desirable activity by contacting a DNA library with a fluorescent labeled DNA probe under conditions permissive of hybridization so as to produce a double-stranded complex of probe and members of the DNA library.

The present invention offers the ability to screen for many types of bioactivities. For instance, the ability to select and combine desired components from a library of polyketides and postpolyketide biosynthesis genes for generation of novel polyketides for study is appealing. The method(s) of the present invention make it possible to and facilitate the cloning of novel polyketide synthase genes and/or gene pathways, and other relevant pathways or genes encoding commercially relevant secondary metabolites, since one can generate gene banks with clones containing large inserts (especially when using vectors which can accept large inserts, such as the f-factor based vectors), which facilitates cloning of gene clusters.

The biopanning approach described above can be used to create libraries enriched with clones carrying sequences substantially homologous to a given probe sequence. Using this approach libraries containing clones with inserts of up to 40 kbp or larger can be enriched approximately 1,000 fold after each round of panning. This enables one to reduce the number of clones to be screened after 1 round of biopanning enrichment. This approach can be applied to create libraries enriched for clones carrying sequence of interest related to a bioactivity of interest, for example, polyketide sequences.

Hybridization screening using high density filters or biopanning has proven an efficient approach to detect homologues of pathways containing genes of interest to discover novel bioactive molecules that may have no known counterparts. Once a polynucleotide of interest is enriched in a library of clones it may be desirable to screen for an activity. For example, it may be desirable to screen for the expression of small molecule ring structures or “backbones”. Because the genes encoding these polycyclic structures can often be expressed in E. coli, the small molecule backbone can be manufactured, even if in an inactive form. Bioactivity is conferred upon transferring the molecule or pathway to an appropriate host that expresses the requisite glycosylation and methylation genes that can modify or “decorate” the structure to its active form. Thus, even if inactive ring compounds, recombinantly expressed in E. coli are detected to identify clones which are then shuttled to a metabolically rich host, such as Streptomyces (e.g., Streptomyces diversae or venezuelae) for subsequent production of the bioactive molecule. It should be understood that E. coli can produce active small molecules and in certain instances it may be desirable to shuttle clones to a metabolically rich host for “decoration” of the structure, but not required. The use of high throughput robotic systems allows the screening of hundreds of thousands of clones in multiplexed arrays in microtiter dishes.

One approach to detect and enrich for clones carrying these structures is to use FACS screening, a procedure described and exemplified in U.S. Ser. No. 08/876,276, filed Jun. 16, 1997. Polycyclic ring compounds typically have characteristic fluorescent spectra when excited by ultraviolet light. Thus, clones expressing these structures can be distinguished from background using a sufficiently sensitive detection method. High throughput FACS screening can be utilized to screen for small molecule backbones in, for example, E. coli libraries. Commercially available FACS machines are capable of screening up to 100,000 clones per second for UV active molecules. These clones can be sorted for further FACS screening or the resident plasmids can be extracted and shuttled to Streptomyces for activity screening.

In another aspect, a bioactivity or biomolecule or compound is detected by using various electromagnetic detection devices, including, for example, optical, magnetic and thermal detection associated with a flow cytometer. Flow cytometer typically use an optical method of detection (fluorescence, scatter, and the like) to discriminate individual cells or particles from within a large population. There are several non-optical technologies that could be used alone or in conjunction with the optical methods to enable new discrimination/screening paradigms.

Magnetic field sensing is one such techniques that can be used as an alternative or in conjunction with, for example, fluorescence based methods. Hall-Effect Sensors are one example of sensors that can be employed. Superconducting Quantum Interference Devices (“SQUIDS”) are the most sensitive sensors for magnetic flux and magnetic fields, so far developed. A standardized criteria for the sensitivity of a SQUID is its energy resolution. This is defined as the smallest change in energy that the SQUID can detect in one second (or in a bandwidth of 1 Hz). Typical values are 10⁻³³ J/Hz. The utility of SQUIDS can be found in the presence of magnetosomes in certain types of bacterial that contain chains of permanent single magnetic domain particles of magnetite (FE₃O₄) of gregite (Fe₃S₄). The magnetic field (or residual magnetic field) of a cell that contains a magnetosome is detected by positioning a SQUID in close proximity to the flow stream of a flow cytometer. Using this method cells or cells containing, for example, magnetic probes can be isolated based on their magnetic properties. As another example, changes in the synthetic pathway of magnetosome containing bacteria can be measured using a similar technique. Such techniques can be used to identify agents which modulate the synthetic pathway of magnetosomes.

Measuring dynamic charge properties is another techniques that can be used as an alternative or in conjunction with, for example, fluorescence based methods. Multipole Coupling Spectroscopy (“MCS”) directly measures the dynamic charge properties of systems without the need for labeling. Structural changes that occur when molecules interact result in representative changes in charge distribution, and these produce a dielectric based spectra or “signature” that reveals the affinity, specificity and functionality of each interaction. Similar changes in charge distribution occur in cellular systems. By observing the changes in these signatures, the dynamics of molecular pathways and cellular function can be resolved in their native conditions. MCS utilizes a small microwave (500 MHz to 50 GHz) transceiver that could be positioned in close proximity to the flow stream of a flow cytometer. Because of the short measurement times (e.g., microseconds) required, a complete MCS signature for each cell within the stream of a flow cytometer can be generated and analyzed. Certain cells can then be sorted and/or isolated based on either spectral features that are known a priori or based on some statistical variation from a general population. Examples of uses for this technique include selection of expression mutants, small molecule pre-screening, and the like.

In one screening approach, biomolecules from candidate clones can be tested for bioactivity by susceptibility screening against test organisms such as Staphylococcus aureus, Micrococcus luteus, E. coli, or Saccharomyces cerevisiae. FACS screening can be used in this approach by co-encapsulating clones with the test organism.

An alternative to the above-mentioned screening methods provided by the present invention is an approach termed “mixed extract” screening. The “mixed extract” screening approach takes advantage of the fact that the accessory genes needed to confer activity upon the polycyclic backbones are expressed in metabolically rich hosts, such as Streptomyces, and that the enzymes can be extracted and combined with the backbones extracted from E. coli clones to produce the bioactive compound in vitro. Enzyme extract preparations from metabolically rich hosts, such as Streptomyces strains, at various growth stages are combined with pools of organic extracts from E. coli libraries and then evaluated for bioactivity. Another approach to detect activity in the E. coli clones is to screen for genes that can convert bioactive compounds to different forms. For example, a recombinant enzyme was recently discovered that can convert the low value daunomycin to the higher value doxorubicin. Similar enzyme pathways are being sought to convert penicillins to cephalosporins.

Screening may be carried out to detect a specified enzyme activity by procedures known in the art. For example, enzyme activity may be screened for one or more of the six IUB classes; oxidoreductases, transferases, hydrolases, lyases, isomerases and ligases. The recombinant enzymes which are determined to be positive for one or more of the IUB classes may then be rescreened for a more specific enzyme activity. Alternatively, the library may be screened for a more specialized enzyme activity. For example, instead of generically screening for hydrolase activity, the library may be screened for a more specialized activity, i.e. the type of bond on which the hydrolase acts. Thus, for example, the library may be screened to ascertain those hydrolases which act on one or more specified chemical functionalities, such as: (a) amide (peptide bonds), i.e. proteases; (b) ester bonds, i.e. esterases and lipases; (c) acetals, i.e., glycosidases.

FACS screening can also be used to detect expression of UV fluorescent molecules in any host, including metabolically rich hosts, such as Streptomyces. For example, recombinant oxytetracylin retains its diagnostic red fluorescence when produced heterologously in S. lividans TK24. Pathway clones, which can be sorted by FACS, can thus be screened for polycyclic molecules in a high throughput fashion.

Recombinant bioactive compounds can also be screened in vivo using “two-hybrid” systems, which can detect enhancers and inhibitors of protein-protein or other interactions such as those between transcription factors and their activators, or receptors and their cognate targets. In this aspect, both the small molecule pathway and the reporter construct are co-expressed. Clones altered in reporter expression can then be sorted by FACS and the pathway clone isolated for characterization.

As indicated, common approaches to drug discovery involve screening assays in which disease targets (macromolecules implicated in causing a disease) are exposed to potential drug candidates which are tested for therapeutic activity. In other approaches, whole cells or organisms that are representative of the causative agent of the disease, such as bacteria or tumor cell lines, are exposed to the potential candidates for screening purposes. Any of these approaches can be employed with the present invention.

The present invention also allows for the transfer of cloned pathways derived from uncultivated samples into metabolically rich hosts for heterologous expression and downstream screening for bioactive compounds of interest using a variety of screening approaches briefly described above.

Recovering Desirable Bioactivities

In one aspect, after viable or non-viable cells, each containing a different expression clone from the gene library are screened, and positive clones are recovered, DNA can be isolated from positive clones utilizing techniques well known in the art. The DNA can then be amplified either in vivo or in vitro by utilizing any of the various amplification techniques known in the art. In vivo amplification would include transformation of the clone(s) or subclone(s) into a viable host, followed by growth of the host. In vitro amplification can be performed using techniques such as the polymerase chain reaction. Once amplified the identified sequences can be “evolved” or sequenced.

Evolution

In one aspect, the present invention manipulates the identified polynucleotides to generate and select for encoded variants with altered activity or specificity. Clones found to have the bioactivity for which the screen was performed can be subjected to directed mutagenesis to develop new bioactivities with desired properties or to develop modified bioactivities with particularly desired properties that are absent or less pronounced in the wild-type activity, such as stability to heat or organic solvents. Any of the known techniques for directed mutagenesis are applicable to the invention. For example, mutagenesis techniques for use in accordance with the invention include those described below.

Alternatively, it may be desirable to variegate a polynucleotide sequence obtained, identified or cloned as described herein. Such variegation can modify the polynucleotide sequence in order to modify (e.g., increase or decrease) the encoded polypeptide's activity, specificity, affinity, function, etc. Such evolution methods are known in the art or described herein, such as, shuffling, cassette mutagenesis, recursive ensemble mutagenesis, sexual PCR, directed evolution, exonuclease-mediated reassembly, codon site-saturation mutagenesis, amino acid site-saturation mutagenesis, gene site saturation mutagenesis, introduction of mutations by non-stochastic polynucleotide reassembly methods, synthetic ligation polynucleotide reassembly, gene reassembly, oligonucleotide-directed saturation mutagenesis, in vivo reassortment of polynucleotide sequences having partial homology, naturally occurring recombination processes which reduce sequence complexity, and any combination thereof.

The clones enriched for a desired polynucleotide sequence, which are identified as described above, may be sequenced to identify the DNA sequence(s) present in the clone, which sequence information can be used to screen a database for similar sequences or functional characteristics. Thus, in accordance with the present invention it is possible to isolate and identify: (i) DNA having a sequence of interest (e.g., a sequence encoding an enzyme having a specified enzyme activity), (ii) associate the sequence with known or unknown sequence in a database (e.g., database sequence associated with an enzyme having an activity (including the amino acid sequence thereof)), and (iii) produce recombinant enzymes having such activity.

Sequencing may be performed by high through-put sequencing techniques. The exact method of sequencing is not a limiting factor of the invention. Any method useful in identifying the sequence of a particular cloned DNA sequence can be used. In general, sequencing is an adaptation of the natural process of DNA replication. Therefore, a template (e.g., the vector) and primer sequences are used. One general template preparation and sequencing protocol begins with automated picking of bacterial colonies, each of which contains a separate DNA clone which will function as a template for the sequencing reaction. The selected clones are placed into media, and grown overnight. The DNA templates are then purified from the cells and suspended in water. After DNA quantification, high-throughput sequencing is performed using a sequencer, such as Applied Biosystems, Inc., Prism 377 DNA Sequencers. The resulting sequence data can then be used in additional methods, including searching a database or databases.

Database Searches and Alignment Algorithms

A number of source databases are available that contain either a nucleic acid sequence and/or a deduced amino acid sequence for use with the invention in identifying or determining the activity encoded by a particular polynucleotide sequence. All or a representative portion of the sequences (e.g., about 100 individual clones) to be tested are used to search a sequence database (e.g., GenBank, PFAM or ProDom), either simultaneously or individually. A number of different methods of performing such sequence searches are known in the art. The databases can be specific for a particular organism or a collection of organisms. For example, there are databases for the C. elegans, Arabadopsis. sp., M. genitalium, M. jannaschii, E. coli, H. influenzae, S. cerevisiae and others. The sequence data of the clone is then aligned to the sequences in the database or databases using algorithms designed to measure homology between two or more sequences.

Such sequence alignment methods include, for example, BLAST (Altschul et al., 1990), BLITZ (MPsrch) (Sturrock & Collins, 1993), and FASTA (Person & Lipman, 1988). The probe sequence (e.g., the sequence data from the clone) can be any length, and will be recognized as homologous based upon a threshold homology value. The threshold value may be predetermined, although this is not required. The threshold value can be based upon the particular polynucleotide length. To align sequences a number of different procedures can be used. Typically, Smith-Waterman or Needleman-Wunsch algorithms are used. However, as discussed faster procedures such as BLAST, FASTA, PSI-BLAST can be used.

For example, optimal alignment of sequences for aligning a comparison window may be conducted by the local homology algorithm of Smith (Smith and Waterman, Adv Appl Math, 1981; Smith and Waterman, J Teor Biol, 1981; Smith and Waterman, J Mol Biol, 1981; Smith et al, J Mol Evol, 1981), by the homology alignment algorithm of Needleman (Needleman and Wuncsch, 1970), by the search of similarity method of Pearson (Pearson and Lipman, 1988), by computerized implementations of these algorithms (GAP, BESTFIT, FASTA, and TFASTA in the Wisconsin Genetics Software Package Release 7.0, Genetics Computer Group, 575 Science Dr., Madison, Wis., or the Sequence Analysis Software Package of the Genetics Computer Group, University of Wisconsin, Madison, Wis.), or by inspection, and the best alignment (i.e., resulting in the highest percentage of homology over the comparison window) generated by the various methods is selected. The similarity of the two sequence (i.e., the probe sequence and the database sequence) can then be predicted.

Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

One example of an algorithm used in the methods of the invention is BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands.

The BLAST algorithm also performs a statistical analysis of the similarity between two sequences (see, e.g., Karlin & Altschul, Proc. Natl. Acad. Sci. USA 90:5873 (1993)). One measure of similarity provided by BLAST algorithm is the smallest sum probability (P(N)), which provides an indication of the probability by which a match between two nucleotide sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

Sequence homology means that two polynucleotide sequences are homologous (i.e., on a nucleotide-by-nucleotide basis) over the window of comparison. A percentage of sequence identity or homology is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence homology. This substantial homology denotes a characteristic of a polynucleotide sequence, wherein the polynucleotide comprises a sequence having at least 60 percent sequence homology, typically at least 70 percent homology, often 80 to 90 percent sequence homology, and most commonly at least 99 percent sequence homology as compared to a reference sequence of a comparison window of at least 25-50 nucleotides, wherein the percentage of sequence homology is calculated by comparing the reference sequence to the polynucleotide sequence which may include deletions or additions which total 20 percent or less of the reference sequence over the window of comparison.

Sequences having sufficient homology can then be further identified by any annotations contained in the database, including, for example, species and activity information. Accordingly, in a typical mixed population sample, a plurality of nucleic acid sequences will be obtained, cloned, sequenced and corresponding homologous sequences from a database identified. This information provides a profile of the polynucleotides present in the sample, including one or more features associated with the polynucleotide including the organism and activity associated with that sequence or any polypeptide encoded by that sequence based on the database information. As used herein “fingerprint” or “profile” refers to the fact that each sample will have associated with it a set of polynucleotides characteristic of the sample and the environment from which it was derived. Such a profile can include the amount and type of sequences present in the sample, as well as information regarding the potential activities encoded by the polynucleotides and the organisms from which polynucleotides were derived. This unique pattern is each sample's profile or fingerprint.

In some instances it may be desirable to express a particular cloned polynucleotide sequence once its identity or activity is determined or a demonstrated identity or activity is associated with the polynucleotide. In such instances the desired clone, if not already cloned into an expression vector, is ligated downstream of a regulatory control element (e.g., a promoter or enhancer) and cloned into a suitable host cell. Expression vectors are commercially available along with corresponding host cells for use in the invention.

As representative examples of expression vectors which may be used there may be mentioned viral particles, baculovirus, phage, plasmids, phagemids, cosmids, fosmids, bacterial artificial chromosomes, viral nucleic acid (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus, yeast, etc.) Thus, for example, the DNA may be included in any one of a variety of expression vectors for expressing a polypeptide. Such vectors include chromosomal, nonchromosomal and synthetic DNA sequences. Large numbers of suitable vectors are known to those of skill in the art, and are commercially available. The following vectors are provided by way of example; ZAP Express, Lambda ZAP®-CMV, Lambda ZAP® II, Lambda gt10, Lambda gt11, pMyr, pSos, pCMV-Script, pCMV-Script XR, pBK Phagemid, pBK-CMV, pBK-RSV, pBluescript II Phagemid, pBluescript II KS+, pBluescript II SK+, pBluescript II SK−, Lambda FIX II, Lambda DASH II, Lambda EMBL3 and EMBL4, EMBL3, EMBL4, SuperCos I and pWE15, pWE15, SuperCos I, pPCR-Script Amp, pPCR-Script Cam, pCMV-Script, pBC KS+, pBC KS−, pBC SK+, pBC SK−, psiX174, pNH8A, pNH16a, pNH18A, pNH46A (Stratagene); PT7BLUE, pSTBlue, pCITE, pET, ptriEx, pForce (Novagen); pIND-E, pIND Vector, pIND/Hygro, pIND(SP1)/Hygro, pIND/GFP, pIND(SP1)/GFP, pIND/V5-His and pIND(SP1)/V5-His Tag, pIND TOPO TA, pShooter™ Targeting Vectors, pTracer™ GFP Reporter Vectors, pcDNA© Vector Collection, EBV Vectors, Voyager™ VP22 Vectors, pVAX1-DNA vaccine vector, pcDNA4/His-Max, pBC1 Mouse Milk System (Invitrogen); pQE70, pQE60, pQE-9, pQE-16, pQE-30/pQE-80, pQE 31/pQE 81, pQE-32/pQE 82, pQE-40, pQE-100 Double Tag (Qiagen); pTRC99a, pKK223-3, pKK233-3, pDR540, pRIT5, pWLNEO, pSV2CAT, pOG44, pXT1, pSG (Stratagene), pSVK3, pBPV, pMSG, pSVL (Pharmacia). However, any other plasmid or vector may be used as long as they are replicable and viable in the host.

The nucleic acid sequence in the expression vector is operatively linked to an appropriate expression control sequence(s) (promoter) to direct mRNA synthesis. Particular named bacterial promoters include lacI, lacZ, T3, T7, gpt, lambda PR, PL, SP6, trp, lacUV5, PBAD, araBAD, araB, trc, proU, p-D-HSP, HSP, GAL4 UAS/E1b, TK, GAL1, CMV/TetO₂ Hybrid, EF-1a CMV, EF-1a CMV, EF-1a CMV, EF, EF-1a, ubiquitin C, rsv-ltr, rsv, b-lactamase, nmt1, and gal10. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector also contains a ribosome binding site for translation initiation and a transcription terminator. The vector may also include appropriate sequences for amplifying expression. Promoter regions can be selected from any desired gene using CAT (chloramphenicol transferase) vectors or other vectors with selectable markers.

In addition, the expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The nucleic acid sequence(s) selected, cloned and sequenced as hereinabove described can additionally be introduced into a suitable host to prepare a library which is screened for the desired enzyme activity. The selected nucleic acid is preferably already in a vector which includes appropriate control sequences whereby a selected nucleic acid encoding an enzyme may be expressed, for detection of the desired activity. The host cell can be a higher eukaryotic cell, such as a mammalian cell, or a lower eukaryotic cell, such as a yeast cell, or the host cell can be a prokaryotic cell, such as a bacterial cell. The selection of an appropriate host is deemed to be within the scope of those skilled in the art from the teachings herein.

In some instances it may be desirable to perform an amplification of the nucleic acid sequence present in a sample or a particular clone that has been isolated. In this aspect the nucleic acid sequence is amplified by PCR reaction or multiple displacement amplification or similar reaction known to those of skill in the art. Commercially available amplification kits are available to carry out such amplification reactions.

In addition, it is important to recognize that the alignment algorithms and searchable database can be implemented in computer hardware, software or a combination thereof. Accordingly, the isolation, processing and identification of nucleic acid sequences and the corresponding polypeptides encoded by those sequence can be implemented in and automated system.

Naked Biopanning involves the direct screening or enrichment for a gene or gene cluster from environmental genomic DNA. The enrichment for or isolation of the desired genomic DNA is performed prior to any cloning, gene-specific PCR or any other procedure that may introduce unwanted bias affecting downstream processing and applications due to toxicity or other issues. Several methodologies can be described for this type of sequence based discovery. These generally include the use of nucleic acid probe(s) that is(are) partially or completely homologous to the target sequence in conjunction with the binding of the probe-target complex to a solid phase support. The probe(s) may be polynucleotide or modified nucleic acid, such as peptide nucleic acid (PNA) and may be used with other facilitating elements such as proteins or additional nucleic acids in the capture of target DNA. An amplification step which does not introduce sequence bias may be used to ensure adequate yield for downstream applications.

An example of a Naked Biopanning approach can be found in the use of RecA protein and a complement-stabilized D-loop (csD-loop) structure (Jayasena & Johnston, 1993; Sena and Zarling, 1993) to target genomic DNA of interest. It does not involve complete denaturation of the target DNA and therefore is of particular interest when one is attempting to capture large genomic fragments. The following method incorporates the ClonCapture™ cDNA selection procedure (CLONTECH Laboratories, Inc.), with some modification, to take advantage of csD-loop formation, a stable structure which may be used to capture genomic DNA containing an internal target sequence.

Environmental genomic DNA is cleaved into fragments (fragment size depends upon type of target and desired downstream insert size if making a pre-enriched library) using mechanical shearing or restriction digest. Fragments are size selected according to desired length and purified. A biotinylated dsDNA probe is produced, based upon existing knowledge of conserved regions within the target, by PCR from a positive clone or by synthetic means. The probe can be internally (ex. incorporation of biotin 21-dCTP) or end labeled with biotin. It must be purified to remove any unincorporated biotin. The probe is heat denatured (5 min. at 95° C.) and placed immediately on ice. The denatured probe is then reacted with RecA and an ATP mix containing ATP and a nonhydrolyzable analog (15 min. at 37° C.). The target DNA is added and incubated with the RecA/biotinylated probe nucleofilaments to form the csD-loop structure (20 min. at 37° C.). The RecA is then removed by treatment with proteinase K and SDS. After inactivating the proteinase K with PMSF, washed and blocked (with sonicated salmon sperm DNA) streptavidin paramagnetic beads are transferred to the reaction and incubated to bind the csD-loop complex to the support (rotate 30 min. at room temp.). The unbound DNA is removed and may be saved for use as target for a different probe. The beads are thoroughly washed and the enriched population is eluted using an alkaline buffer and transferred off. The enriched DNA is then ethanol precipitated and is ready for ligation and pre-enriched library preparation.

Other stable complexes may be used instead of the RecA/csD-loop structure for the capture of genomic DNA. For instance, PNAs may be used, either as “openers” to allow insertion of a probe into dsDNA (Bukanov et al., 1998), or as tandem probes themselves (Lohse et al., 1999). In the first case, PNAs bind to two short tracts of homopurines that are in close proximity to each other. They form P-loop structures, which displace the unbound strand and make it available for binding by a probe, which can then be used to capture the target using an affinity capture method involving a solid phase. Likewise, PNAs may be used in a “double-duplex invasion” to form a stable complex and allow target recovery.

Simpler methods may be used in the retrieval of targets from environmental genomic DNA that involve complete denaturation of the DNA fragments. After cutting genomic DNA into fragments of the desired length via mechanical shearing or through the use of restriction enzymes, the target DNA may be bound to a solid phase using a direct hybridization affinity capture scheme. A nucleic acid probe is covalently bound to a solid phase such as a glass slide, paramagnetic bead, or any type of matrix in a column, and the denatured target DNA is allowed to hybridize to it. The unbound fraction may be collected and re-hybridized to the same probe to ensure a more complete recovery, or to a host of different probes, as a part of a cascade scenario, where a population of environmental genomic DNA is subsequently panned for a number of different genes or gene clusters.

Linkers containing restriction sites and sites for common primers may be added to the ends of the genomic fragments using sticky-ended or blunt-ended ligations (depending upon the method used for cutting the genomic DNA). These enable one to amplify the size-selected inserted fragment population by PCR without significant sequence bias. Thus, after using any of the abovementioned techniques for isolation or enrichment, one may help to ensure adequate recovery for downstream processing. Furthermore, the recovered population is ready for cutting and ligation into a suitable vector as well as containing the priming sites for sequencing at any time.

A variation of the above scheme involves including a tag from a combinatorial synthesis of polynucleotide tags (Brenner et al., 1999) within the linker that is attached onto the ends of the genomic fragments. This allows each fragment within the starting population to have its own unique tag. Therefore, when amplified with common primers, each of these uniquely tagged fragments give rise to a multitude of in vitro clones which are then bound to the paramagnetic bead containing millions of copies of the complementary, covalently bound anti-tag. A fluorescently labeled, target specific probe may be subsequently hybridized to the target-containing beads. The beads may be sorted using FACS, where the positives may be sequenced directly from the beads and the insert may be cut out and ligated into the desired vector for further processing. The negative population may be hybridized with other probes and resorted as part of the cascade scenario previously described.

Transposon technology may allow the insertion of environmental genomic DNA into a host genome through the use of transposomes (Goryshin & Reznikoff, 1998) to avoid bias resulting from expression of toxic genes. The host cells are then cultured to provide more copies of target DNA for discovery, isolation, and downstream processes.

Host cells may be genetically engineered (transduced or transformed or transfected) with the vectors. The engineered host cells can be cultured in conventional nutrient media modified as appropriate for activating promoters, selecting transfonnants or amplifying genes. The culture conditions, such as temperature, pH and the like, are those previously used with the host cell selected for expression, and will be apparent to the ordinarily skilled artisan.

The clones which are identified as having the specified protein, e g. enzyme, activity may then be sequenced to identify the DNA sequence encoding an protein, e.g. enzyme, having the specified activity. Thus, in accordance with the present invention it is possible to isolate and identify: (i) DNA encoding an protein, e.g. enzyme, having a specified protein, e.g. enzyme, activity, (ii) proteins, e.g. enzymes, having such activity (including the amino acid sequence thereof) and (iii) produce recombinant proteins, e.g. enzymes, having such activity.

The present invention may be employed for example, to identify uncultured microorganisms with proteins, e.g. enzymes, having, for example, the following activities which may be employed for the following uses:
1. Lipase/Esterase
   a. Enantioselective hydrolysis of esters (lipids)/thioesters
      1) Resolution of racemic mixtures
      2) Synthesis of optically active acids or alcohols from mesodiesters
   b. Selective syntheses
      1) Regiospecific hydrolysis of carbohydrate esters
      2) Selective hydrolysis of cyclic secondary alcohols
   c. Synthesis of optically active esters, lactones, acids, alcohols
      1) Transesterification of activated/nonactivated esters
      2) Interesterification
      3) Optically active lactones from hydroxyesters
      4) Regio- and enantioselective ring opening of anhydrides
   d. Detergents
   e. Fat/Oil conversion
   f. Cheese ripening
2. Protease
   a. Ester/amide synthesis
   b. Peptide synthesis
   c. Resolution of racemic mixtures of amino acid esters
   d. Synthesis of non-natural amino acids
   e. Detergents/protein hydrolysis
3. Glycosidase/Glycosyl transferase
   a. Sugar/polymer synthesis
   b. Cleavage of glycosidic linkages to form mono, all-and oligosaccharides
   c. Synthesis of complex oligosaccharides
   d. Glycoside synthesis using UDP-galactosyl transferase
   e. Transglycosylation of disaccharides, glycosyl fluorides, aryl galactosides
   f. Glycosyl transfer in oligosaccharide synthesis
   g. Diastereoselective cleavage of p-glucosylsulfoxides
   h. Asymmetric glycosylations
   i. Food processing
   j. Paper processing
4. Phosphatase/Kinase
   a. Synthesis/hydrolysis of phosphate esters
      1) Regio-, enantioselective phosphorylation
      2) Introduction of phosphate esters
      3) Synthesize phospholipid precursors
      4) Controlled polynucleotide synthesis
   b. Activate biological molecule
   c. Selective phosphate bond formation without protecting groups
5. Mono/Dioxygenase
   a. Direct oxyfunctionalization of unactivated organic substrates
   b. Hydroxylation of alkanes, aromatics, steroids
   c. Epoxidation of alkenes
   d. Enantioselective sulphoxidation
   e. Regio- and stereoselective Bayer-Villiger oxidation
6. Haloperoxidase
   a. Oxidative addition of halide ion to nucleophilic sites
   b. Addition of hypohalous acids to olefinic bonds
   c. Ring cleavage of cyclopropanes
   d. Activated aromatic substrates converted to ortho and para derivatives
   e. 1.3 diketones converted to 2-halo-derivatives
   f. Heteroatom oxidation of sulfur and nitrogen containing substrates
   g. Oxidation of enol acetates, alkynes and activated aromatic rings
7. Lignin peroxidase/Diarylpropane peroxidase
   a. Oxidative cleavage of C—C bonds
   b. Oxidation of benzylic alcohols to aldehydes
   c Hydroxylation of benzylic carbons
   d. Phenol dimerization
   e. Hydroxylation of double bonds to form diols
   f. Cleavage of lignin aldehydes
8. Epoxide hydrolase
   a. Synthesis of enantiomerically pure bioactive compounds
   b. Regio- and enantioselective hydrolysis of epoxide Aromatic and olefinic epaxidation by monoaxygenases to form epoxides
   c. Resolution of racemic epoxides
   d. Hydrolysis of steroid epoxides
9. Nitrile hydratase/nitriluse
   a. Hydrolysis of aliphatic nitrites to carboxamides
   b. Hydrolysis of aromatic, heterocyclic, unsaturated aliphatic nitriles to corresponding acids
   c. Hydrolysis of acrylonitrile
   d. Production of aromatic and carboxamides, carboxylic acids (nicotinamide, picolinamide, isonicotinamide)
   e. Regioselective hydrolysis of acrylic dinitrile
   f. α-amino acids from α-hydroxynitriles
10. Transaminase
   a. Transfer of amino groups into oxo-acids
11. Amidase/Acylase
   a. Hydrolysis of amides, amidines, and other C—N bonds
   b. Non-natural amino acid resolution and synthesis

### EXAMPLE 1

### DNA Isolation and Library Construction

The following outlines the procedures used to generate a gene library from a mixed population of organisms.

DNA isolation. DNA is isolated using the IsoQuick Procedure as per manufacturer's instructions (Orca, Research Inc., Bothell, Wash.). DNA can be normalized according to Example 2 below. Upon isolation the DNA is sheared by pushing and pulling the DNA through a 25G double-hub needle and a 1-cc syringes about 500 times. A small amount is run on a 0.8% agarose gel to make sure the majority of the DNA is in the desired size range (about 3-6 kb).

Blunt-ending DNA. The DNA is blunt-ended by mixing 45 ul of 10× Mung Bean Buffer, 2.0 ul Mung Bean Nuclease (150 u/ul) and water to a final volume of 405 ul. The mixture is incubate at 370C for 15 minutes. The mixture is phenol/chloroform extracted followed by an additional chloroform extraction. One ml of ice cold ethanol is added to the final extract to precipitate the DNA. The DNA is precipitated for 10 minutes on ice. The DNA is removed by centrifugation in a microcentrifuge for 30 minutes. The pellet is washed with 1 ml of 70% ethanol and repelleted in the microcentrifuge. Following centrifugation the DNA is dried and gently resuspended in 26 ul of TE buffer.

Methylation of DNA. The DNA is methylated by mixing 4 ul of 10× EcoR I Methylase Buffer, 0.5 ul SAM (32 mM), 5.0 ul EcoR I Methylase (40 u/ul) and incubating at 370C, 1 hour. In order to insure blunt ends, add to the methylation reaction: 5.0 ul of 100 mM MgCl2, 8.0 ul of dNTP mix (2.5 mM of each dGTP, dATP, dTTP, dCTP), 4.0 ul of Klenow (5 u/ul) and incubate at 120C for 30 minutes.

After 30 minutes add 450 ul 1×STE. The mixture is phenol/chloroform extracted once followed by an additional chloroform extraction. One ml of ice cold ethanol is added to the final extract to precipitate the DNA. The DNA is precipitated for 10 minutes on ice. The DNA is removed by centrifugation in a microcentrifuge for 30 minutes. The pellet is washed with 1 ml of 70% ethanol, repelleted in the microcentrifuge and allowed to dry for 10 minutes.

Ligation. The DNA is ligated by gently resuspending the DNA in 8 ul EcoR I adaptors (from Stratagene's cDNA Synthesis Kit), 1.0 ul of 10× Ligation Buffer, 1.0 ul of 10 mM rATP, 1.0 ul of T4 DNA Ligase (4 Wu/ul) and incubating at 4oC for 2 days. The ligation reaction is terminated by heating for 30 minutes at 70oC.

Phosphorylation of adaptors. The adaptor ends are phosphorylated by mixing the ligation reaction with 1.0 ul of 10× Ligation Buffer, 2.0 ul of 10 mM rATP, 6.0 ul of H2O, 1.0 ul of polynucleotide kinase (PNK) and incubating at 37oC for 30 minutes. After 30 minutes 31 ul H2O and 5 ml 10×STE are added to the reaction and the sample is size fractionate on a Sephacryl S-500 spin column. The pooled fractions (1-3) are phenol/chloroform extracted once followed by an additional chloroform extraction. The DNA is precipitated by the addition of ice cold ethanol on ice for 10 minutes. The precipitate is pelleted by centrifugation in a microfuge at high speed for 30 minutes. The resulting pellet is washed with 1 ml 70% ethanol, repelleted by centrifugation and allowed to dry for 10 minutes. The sample is resuspended in 10.5 ul TE buffer. Do not plate. Instead, ligate directly to lambda arms as above except use 2.5 ul of DNA and no water.

Sucrose Gradient (2.2 ml) Size Fractionation. Stop ligation by heating the sample to 65oC for 10 minutes. Gently load sample on 2.2 ml sucrose gradient and centrifuge in mini-ultracentrifuge at 45K, 20oC for 4 hours (no brake). Collect fractions by puncturing the bottom of the gradient tube with a 20G needle and allowing the sucrose to flow through the needle. Collect the first 20 drops in a Falcon 2059 tube then collect 10 1-drop fractions (labeled 1-10). Each drop is about 60 ul in volume. Run 5 ul of each fraction on a 0.8% agarose gel to check the size. Pool fractions 1-4 (about 10-1.5 kb) and, in a separate tube, pool fractions 5-7 (about 5-0.5 kb). Add 1 ml ice cold ethanol to precipitate and place on ice for 10 minutes. Pellet the precipitate by centrifugation in a microfuge at high speed for 30 minutes. Wash the pellets by resuspending them in 1 ml 70% ethanol and repelleting them by centrifugation in a microfuge at high speed for 10 minutes and dry. Resuspend each pellet in 10 ul of TE buffer.

Test Ligation to Lambda Arms. Plate assay by spotting 0.5 ul of the sample on agarose containing ethidium bromide along with standards (DNA samples of known concentration) to get an approximate concentration. View the samples using UV light and estimate concentration compared to the standards. Fraction 1-4=>1.0 ug/ul. Fraction 5-7=500 ng/ul.

Prepare the following ligation reactions (5 μl reactions) and incubate 4oC, overnight:

| | | | | Lambda | | T4 DNA |
|---|---|---|---|---|---|---|
| | | 10X Ligase | 10 mM | arms | Insert | Ligase |
| Sample | H₂O | Buffer | rATP | (ZAP) | DNA | (4 Wu/(l) |
| Fraction 1-4 | 0.5 ul | 0.5 ul | 0.5 ul | 1.0 ul | 2.0 ul | 0.5 ul |
| Fraction 5-7 | 0.5 ul | 0.5 ul | 0.5 ul | 1.0 ul | 2.0 ul | 0.5 ul |

Test Package and Plate. Package the ligation reactions following manufacturer's protocol. Stop packaging reactions with 500 ul SM buffer and pool packaging that came from the same ligation. Titer 1.0 ul of each pooled reaction on appropriate host (OD₆₀₀=1.0) [XLI-Blue MRF]. Add 200 ul host (in mM MgSO₄) to Falcon 2059 tubes, inoculate with 1 ul packaged phage and incubate at 37° C. for 15 minutes. Add about 3 ml 48° C. top agar [50 ml stock containing 150 ul IPTG (0.5M) and 300 ul X-GAL (350 mg/ml)] and plate on 100 mm plates. Incubate the plates at 37° C., overnight.

Amplification of Libraries (5.0×10⁵ recombinants from each library). Add 3.0 ml host cells (OD₆₀₀=1.0) to two 50 ml conical tube and inoculate with 2.5×10⁵ pfu of phage per conical tube. Incubate at 37° C. for 20 minutes. Add top agar to each tube to a final volume of 45 ml. Plate each tube across five 150 mm plates. Incubate the plates at 37° C. for 6-8 hours or until plaques are about pin-head in size. Overlay the plates with 8-10 ml SM Buffer and place at 4° C. overnight (with gentle rocking if possible).

Harvest Phage. Recover phage suspension by pouring the SM buffer off each plate into a 50-ml conical tube. Add 3 ml of chloroform, shake vigorously and incubate at room temperature for 15 minutes. Centrifuge the tubes at 2K rpm for 10 minutes to remove cell debris. Pour supernatant into a sterile flask, add 500 ul chloroform and store at 4° C.

Titer Amplified Library. Make serial dilutions of the harvested phage (for example, 10⁻⁵=1 ul amplified phage in 1 ml SM Buffer; 10⁻⁶=1 ul of the 10⁻³ dilution in 1 ml SM Buffer). Add 200 ul host (in 10 mM MgSO₄) to two tubes. Inoculate one tube with 10 ul 10⁻⁶ dilution (10⁻⁵). Inoculate the other tube with 1 ul 10⁻⁶ dilution (10⁻⁶). Incubate at 37° C. for 15 minutes. Add about 3 ml 48° C. top agar [50 ml stock containing 150 ul IPTG (0.5M) and 375 ul X-GAL (350 mg/ml)] to each tube and plate on 100 mm plates. Incubate the plates at 37° C., overnight. Excise the ZAP II library to create the pBLUESCRIPT library according to manufacturers protocols (Stratagene).

### EXAMPLE 2

### Enzymatic Activity Assay

The following is a representative example of a procedure for screening an expression library prepared in accordance with Example 1. In the following, the chemical characteristic Tiers are as follows:
Tier 1: Hydrolase
Tier 2: Amide, Ester and Acetal
Tier 3: Divisions and subdivisions are based upon the differences between individual substrates that are covalently attached to the functionality of Tier 2 undergoing reaction; as well as substrate specificity.
Tier 4: The two possible enantiomeric products which the protein, e.g. enzyme, may produce from a substrate.

Although the following example is specifically directed to the above-mentioned tiers, the general procedures for testing for various chemical characteristics is generally applicable to substrates other than those specifically referred to in this Example.

Screening for Tier 1-hydrolase; Tier 2-amide. Plates of the library prepared as described in Example 1 are used to multiply inoculate a single plate containing 200 μl of LB Amp/Meth, glycerol in each well. This step is performed using the High Density Replicating Tool (HDRT) of the Beckman Biomek with a 1% bleach, water, isopropanol, air-dry sterilization cycle between each inoculation. The single plate is grown for 2h at 37° C. and is then used to inoculate two white 96-well Dynatech microtiter daughter plates containing 250 μl of LB Arnp/Meth, glycerol in each well. The original single plate is incubated at 37° C. for 18 h, then stored at 80° C. The two condensed daughter plates are incubated at 37° C. also for 18 h. The condensed daughter plates are then heated at 70° C. for 45 min. to kill the cells and inactivate the host E. coli proteins, e.g. enzymes. A stock solution of 5 mg/mL morphourea phenylalanyl-7-amino-4-trifluoromethyl coumarin (MuPheAFC, the ‘substrate’) in DMSO is diluted to 600 μM with 50 mM pH 7.5 Hepes buffer containing 0.6 mg/ml of the detergent dodecyl maltoside.

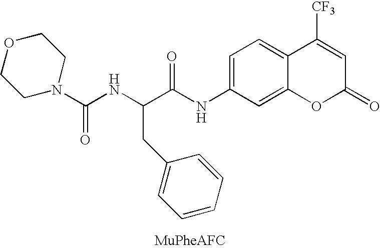

Fifty μl of the 600 μM MuPheAFC solution is added to each of the wells of the white condensed plates with one 100 μl mix cycle using the Biomek to yield a final concentration of substrate of ˜100 μM. The fluorescence values are recorded (excitation 400 nm, emission=505 nm) on a plate reading fluorometer immediately after addition of the substrate (t=O). The plate is incubated at 70° C. for 100 min, then allowed to cool to ambient temperature for 15 additional minutes. The fluorescence values are recorded again (t=100). The values at t=0 are subtracted from the values at t=100 to determine if an active clone is present.

The data will indicate whether one of the clones in a particular well is hydrolyzing the substrate. In order to determine the individual clone which carries the activity, the source library plates are thawed and the individual clones are used to singly inoculate a new plate containing LB Amp/Meth, glycerol. As above, the plate is incubated at 37° C. to grow the cells, heated at 70° C. to inactivate the host proteins, e.g. enzymes, and 50 μl of 600 μM MuPheAFC is added using the Biomek. Additionally three other substrates are tested. They are methyl umbelliferone heptanoate, the CBZ-arginine rhodamine derivative, and fluorescein-conjugated casein (˜3.2 mol fluorescein per mol of casein).

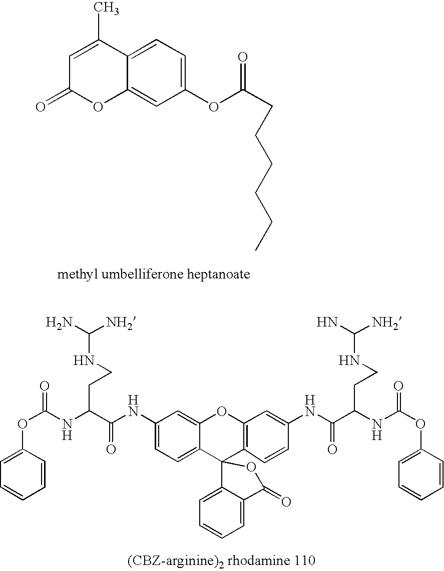

The umbelliferone and rhodamine are added as 600 μM stock solutions in 50 μl of Hepes buffer. The fluorescein-conjugated casein is also added in 50 μl at a stock concentration of 20 and 200 mg/ml. After addition of the substrates the t=0 fluorescence values are recorded, the plate is incubated at 70° C., and the t=100 min. values are recorded as above.

These data indicate which plate the active clone is in, where the arginine rhodamine derivative is also turned over by this activity, but the lipase substrate. methyl umbelliferone heptanoate, and protein, fluorescein-conjugated casein, do not function as substrates, the Tier 1 classification is ‘hydrolase’ and the Tier 2 classification is amide bond. No cross reactivity should be seen with the Tier 2-ester classification.

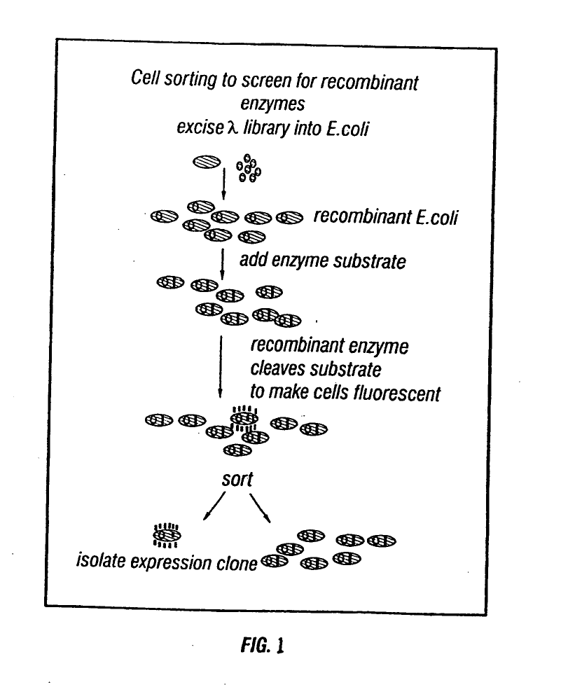

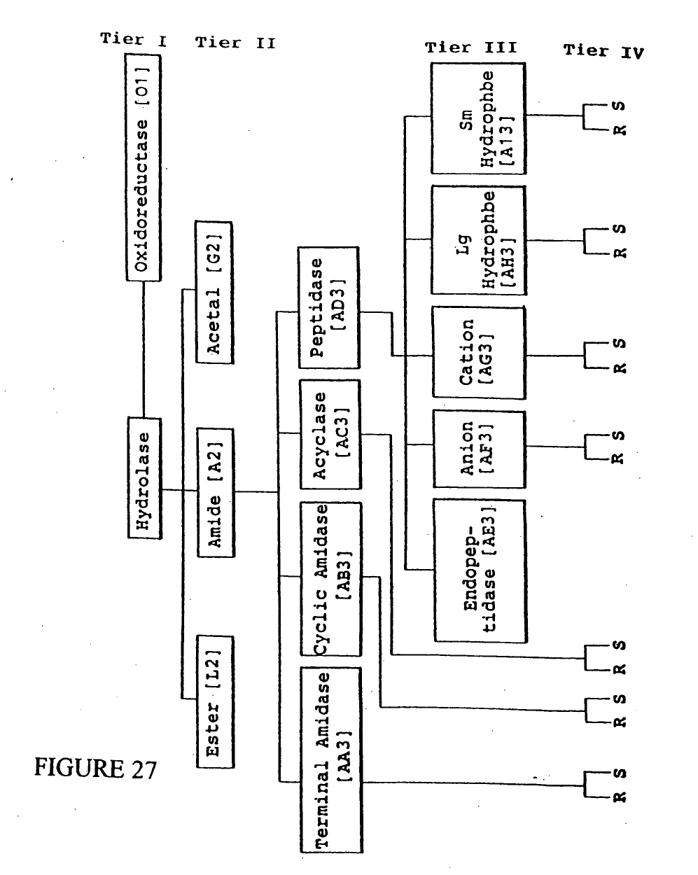

As shown in FIG. 27, a recombinant clone from the library which has been characterized in Tier 1 as hydrolase and in Tier 2 as amide may then be tested in Tier 3 for various specificities. In FIG. 1, the various classes of Tier 3 are followed by a parenthetical code which identifies the substrates of Table 1 which are used in identifying such specificities of Tier 3.

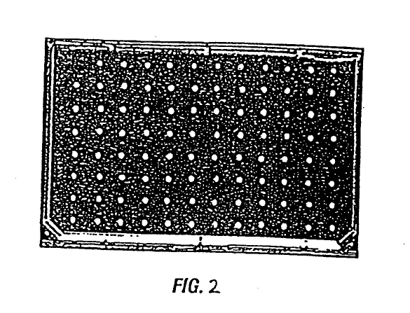

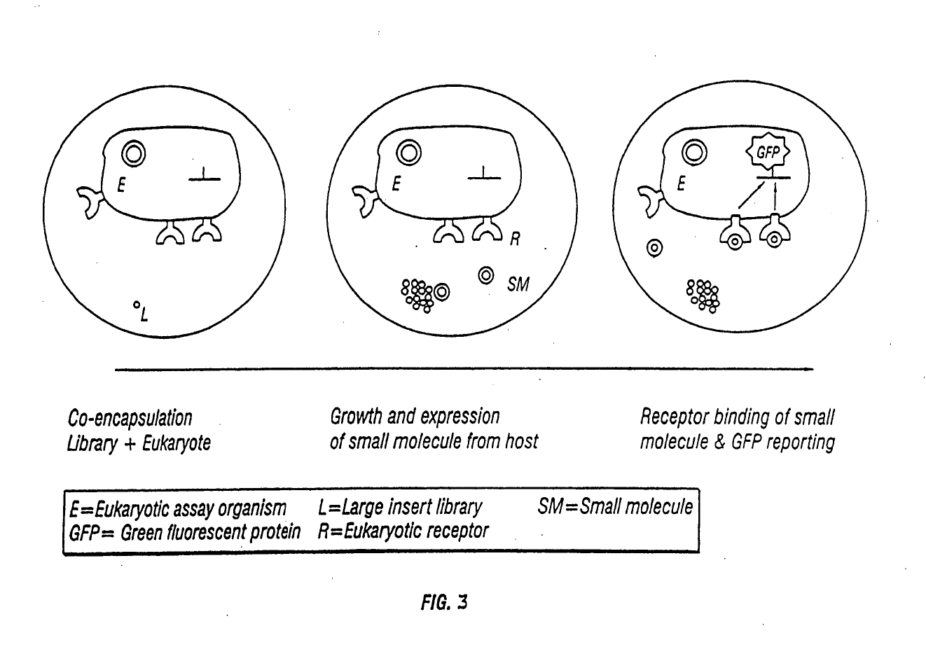

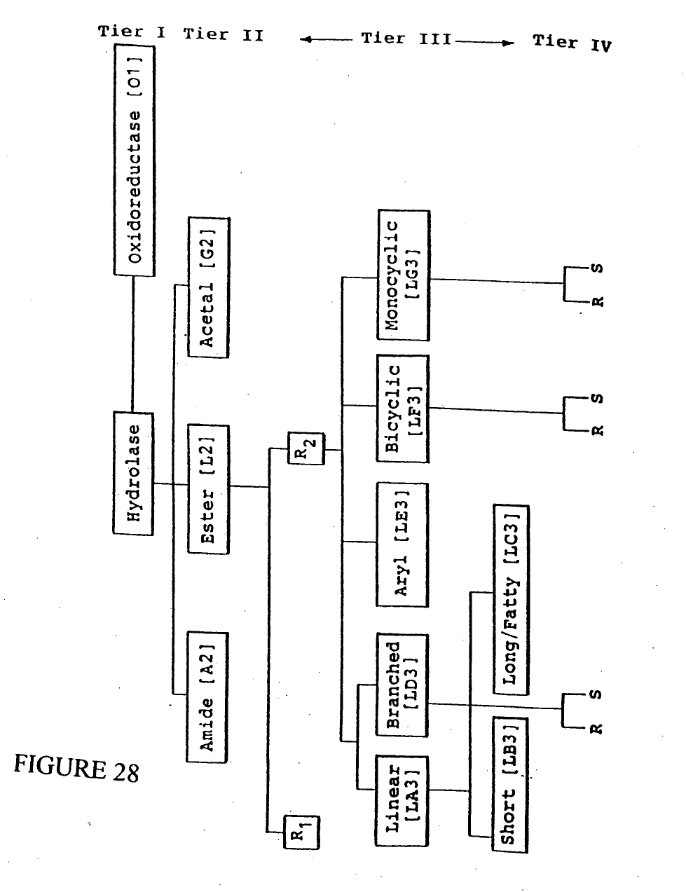

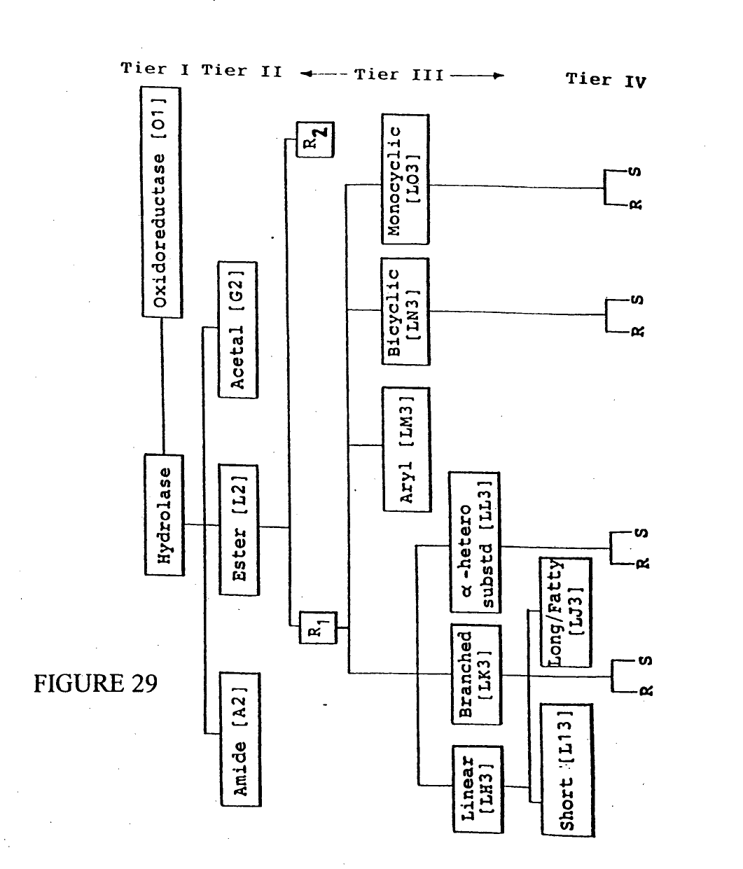

As shown in FIGS. 28 and 29, a recombinant clone from the library which has been characterized in Tier 1 as hydrolase and in Tier 2 as ester may then be tested in Tier 3 for various specificities. In FIGS. 2 and 3, the various classes of Tier 3 are followed by a parenthetical code which identifies the substrates of Tables 3 and 4 which are used in identifying such specificities of Tier 3. In FIGS. 2 and 3, R₂ represents the alcohol portion of the ester and R₁ represents the acid portion of the ester.

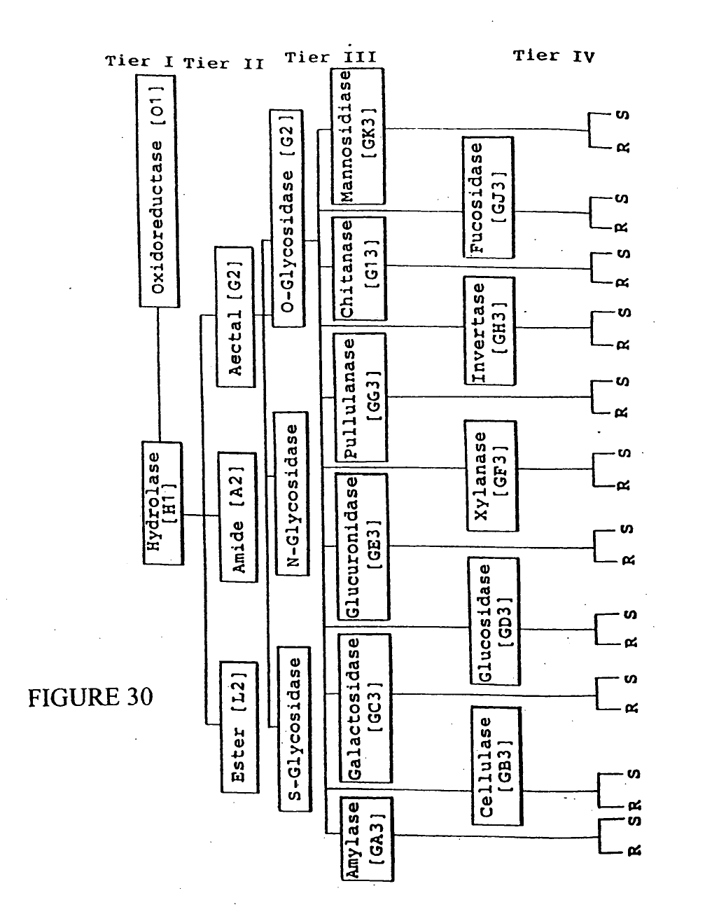

As shown in FIG. 30, a recombinant clone from the library which has been characterized in Tier 1 as hydrolase and in Tier 2 as acetal may then be tested in Tier 3 for various specificities. In FIG. 29, the various classes of Tier 3 are followed by a parenthetical code which identifies the substrates of Table 5 which are used in identifying such specificities of Tier 3.

Proteins, e.g. enzymes, may be classified in Tier 4 for the chirality of the product(s) produced by the enzyme. For example, chiral amino esters may be determined using at least the following substrates:

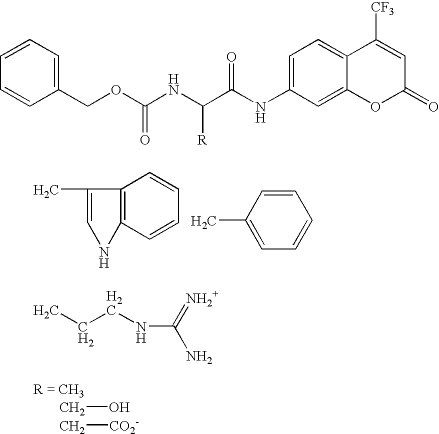

For each substrate which is turned over the enantioselectivity value, E, is determined according to the equation below:

$$E = \frac{ln [ 1 - c ( 1 + {ee}_{p} ) ]}{ln [ 1 - c ( 1 - {ee}_{p} ) ]}$$

where eeₚ=the enantiomeric excess (ee) of the hydrolyzed product and c=the percent conversion of the reaction. See Wong and Whitesides, Proteins, e.g. enzymes, in Synthetic Organic Chemistry, 1994, Elsevier, Tarrytown, N.Y., pp. 9-12.

The enantiomeric excess is determined by either chiral high performance liquid chromatography (HPLC) or chiral capillary electrophoresis (CE). Assays are performed as follows: two hundred μl of the appropriate buffer is added to each well of a 96-well white microtiter plate, followed by 50 μl of partially or completely purified protein, e.g. enzyme, solution; 50 μl of substrate is added and the increase in fluorescence monitored versus time until 50% of the substrate is consumed or the reaction stops, whichever comes first.

### EXAMPLE 3

### Construction of a Stable, Large Insert Picoplankton Genomic DNA Library

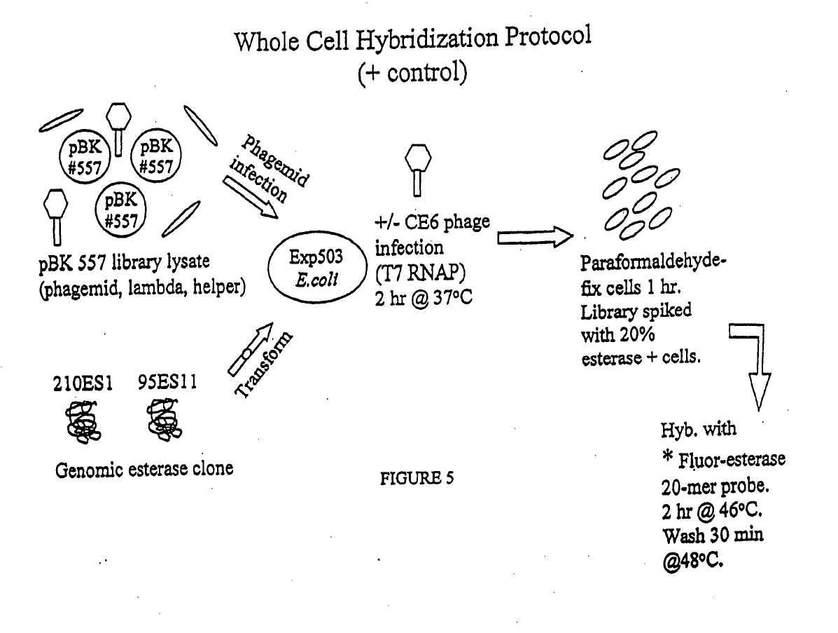

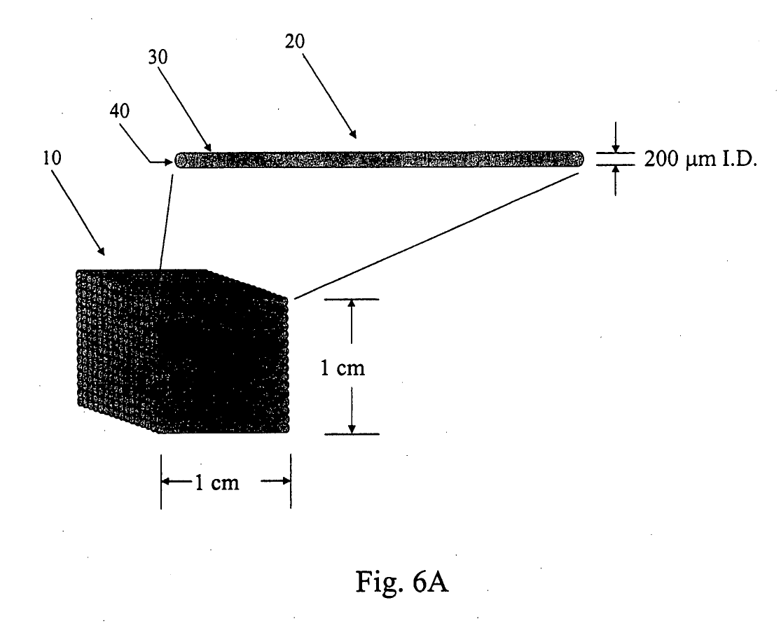

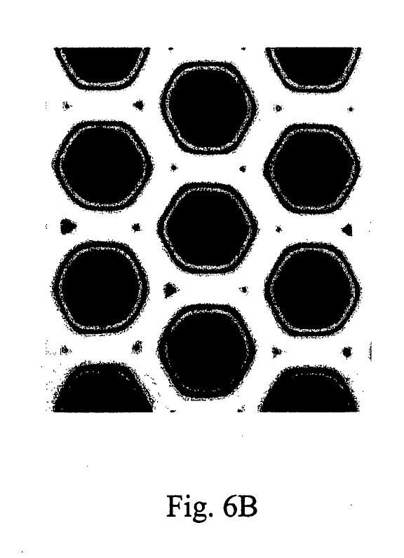

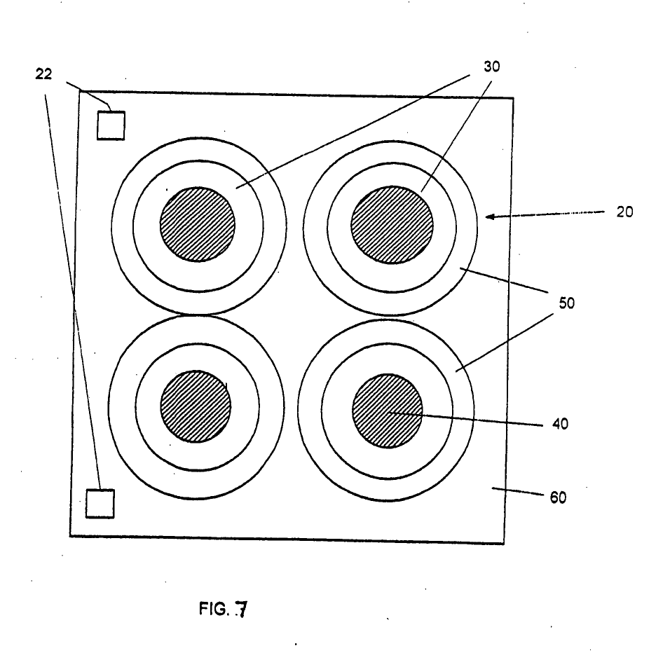

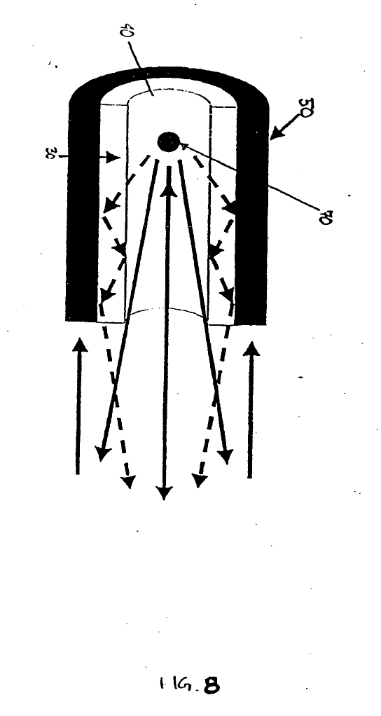

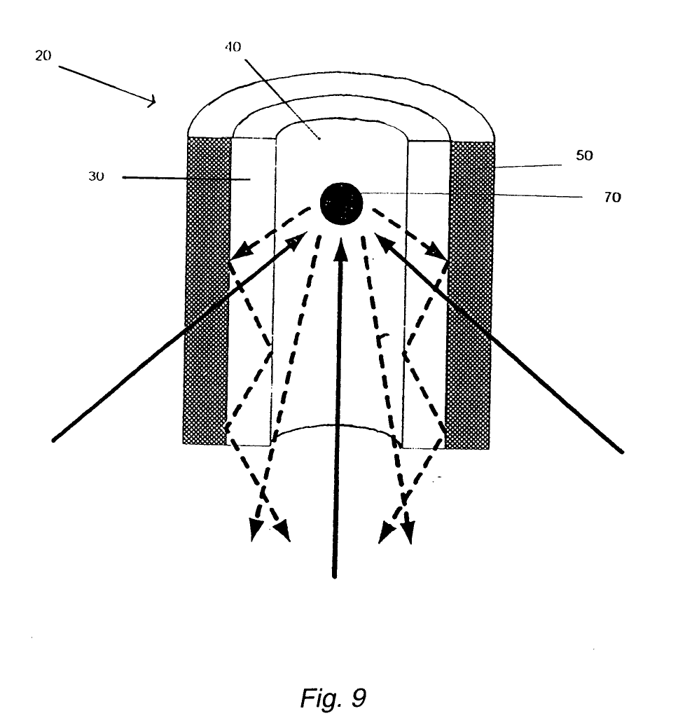

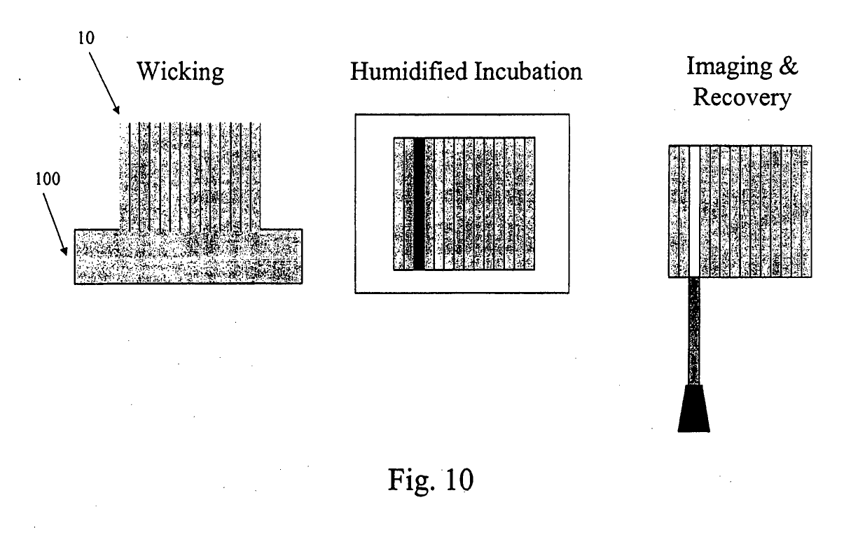

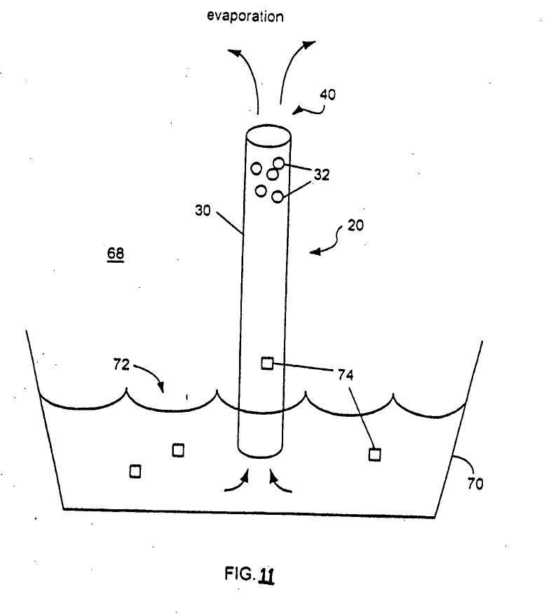

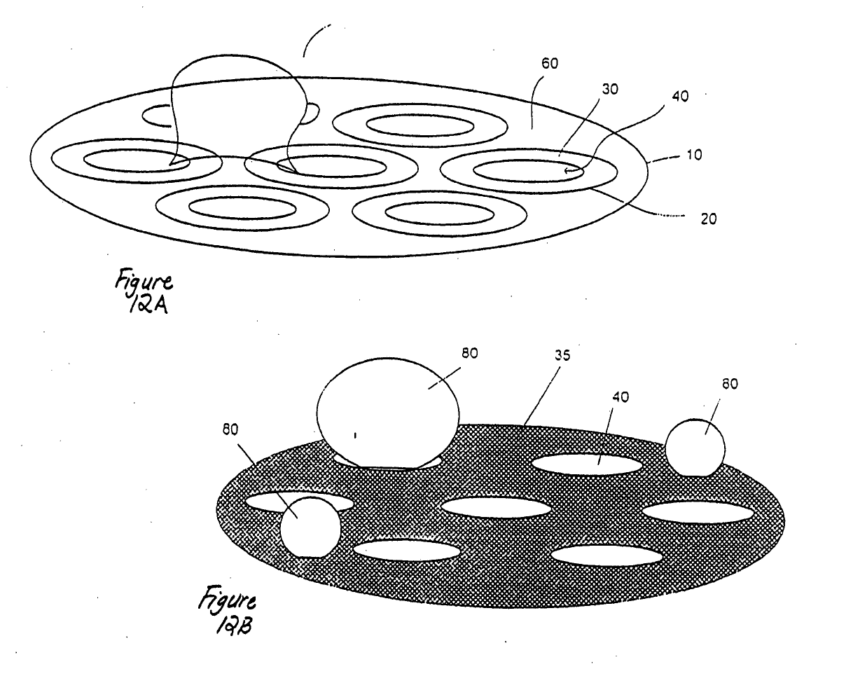

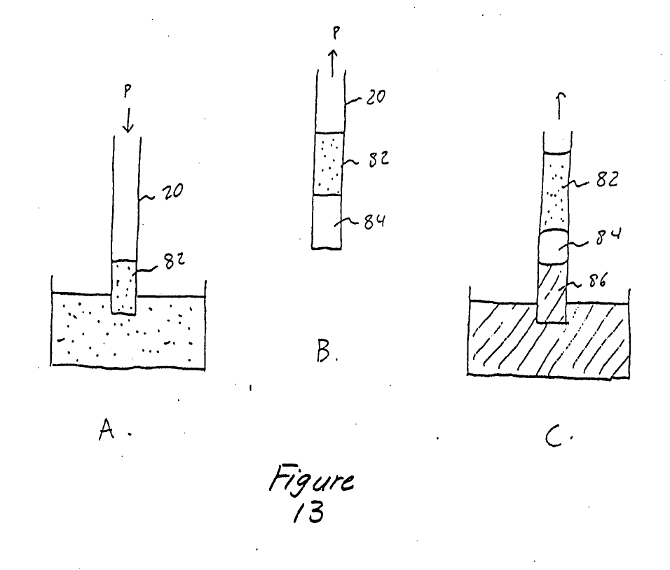

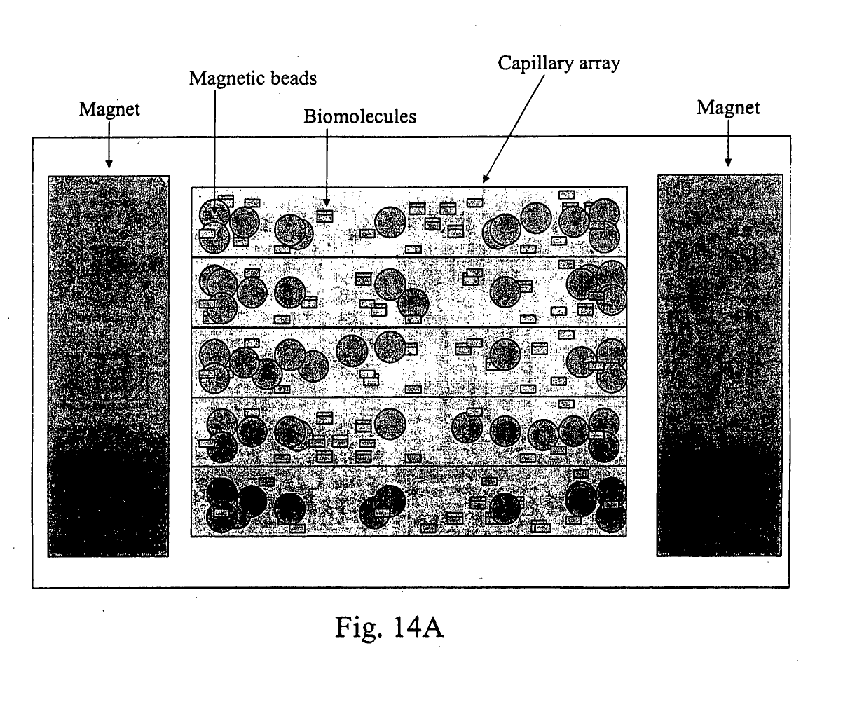

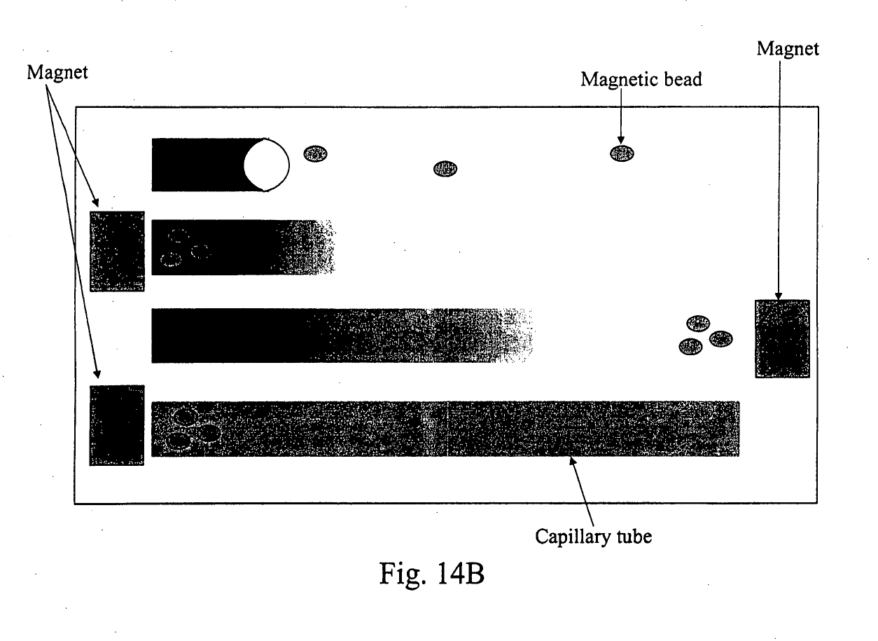

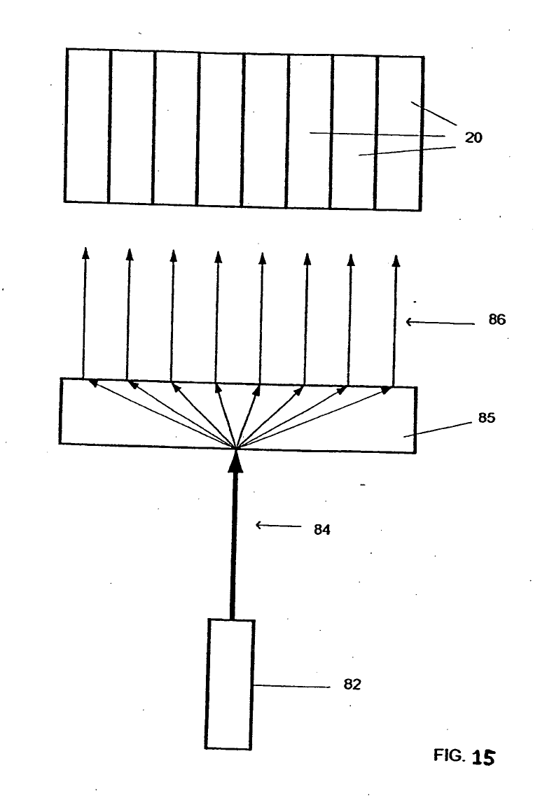

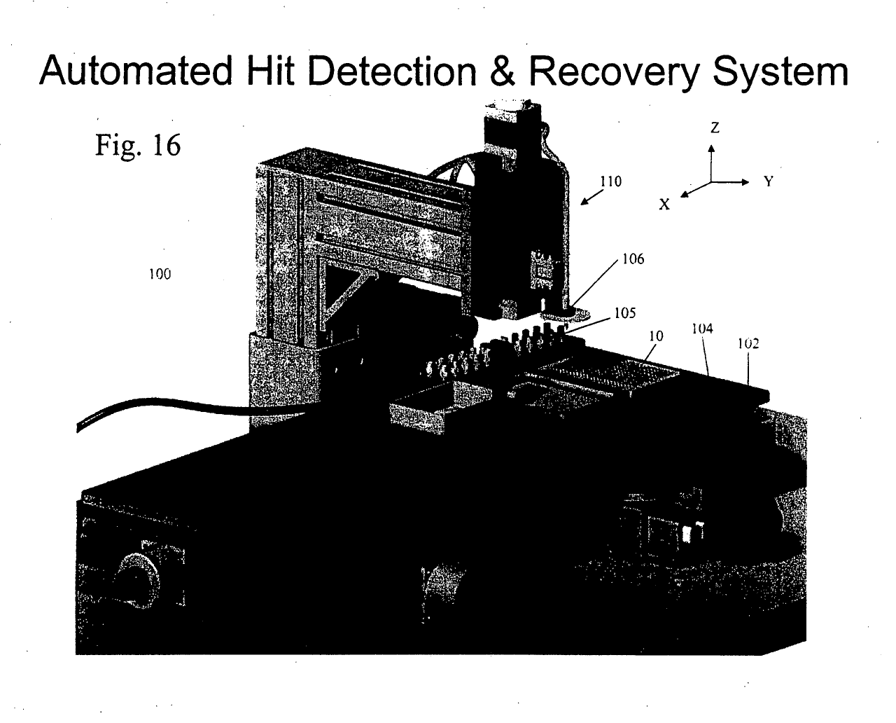

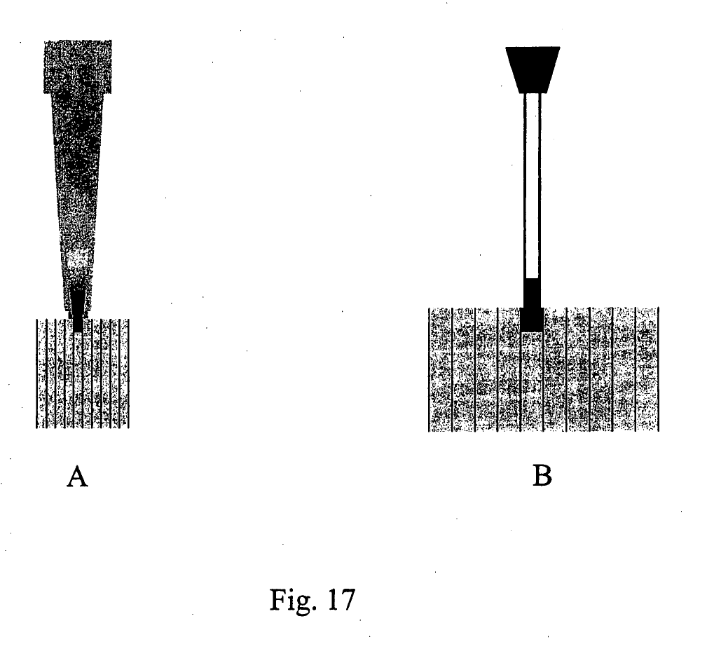

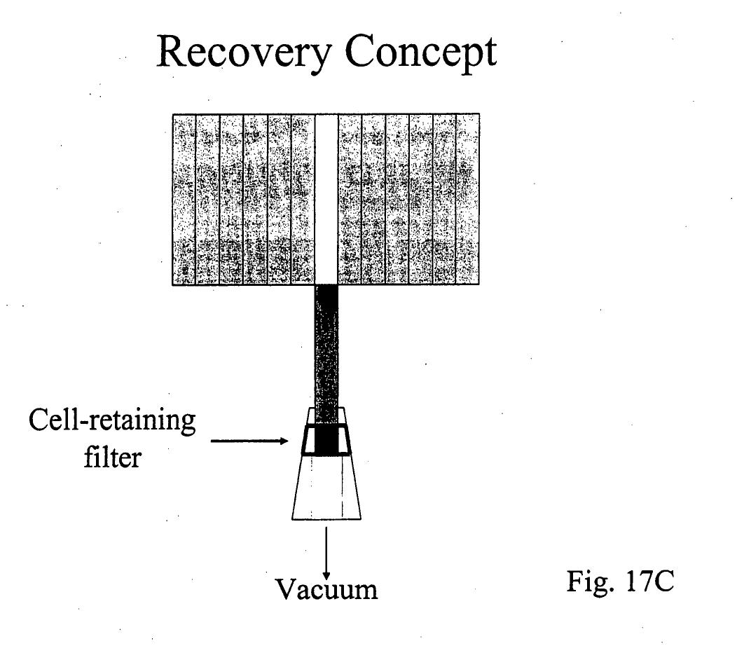

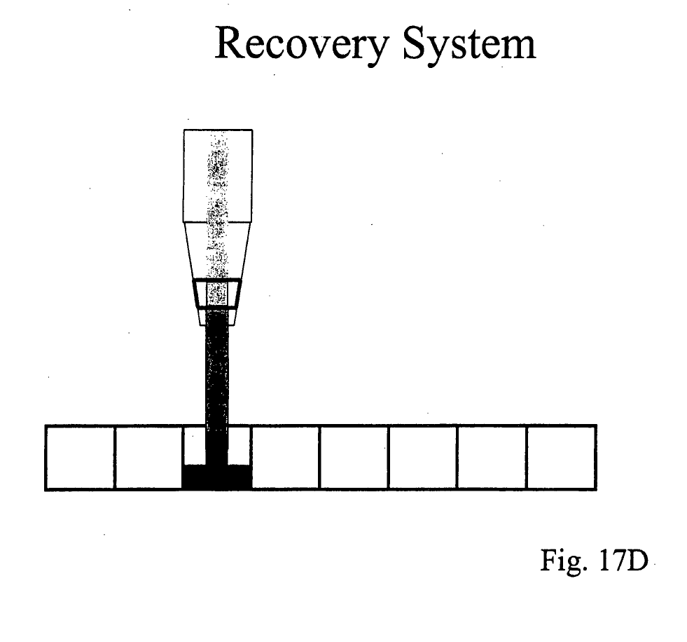

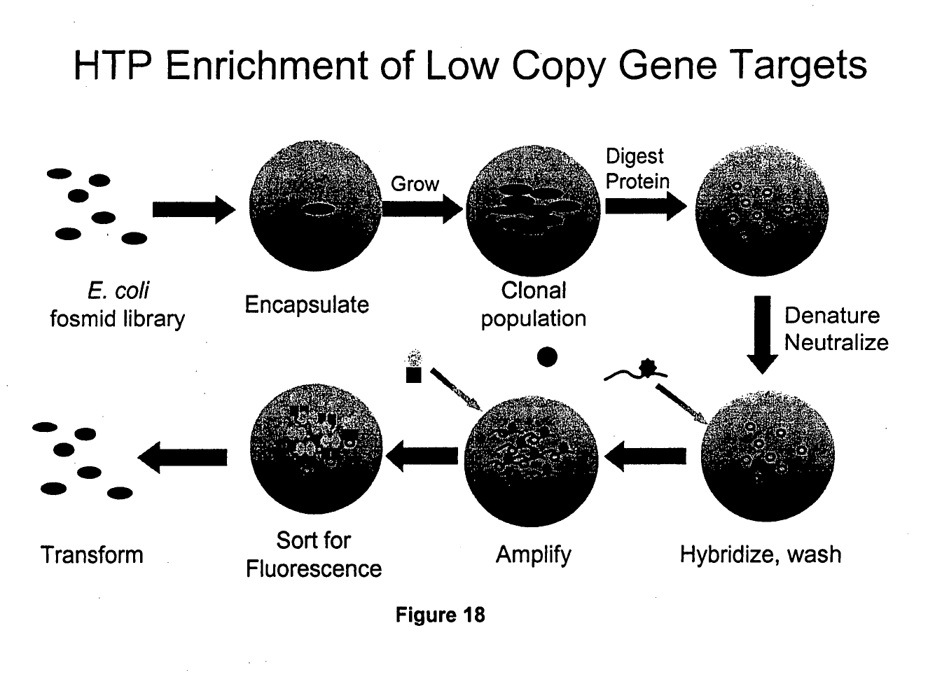

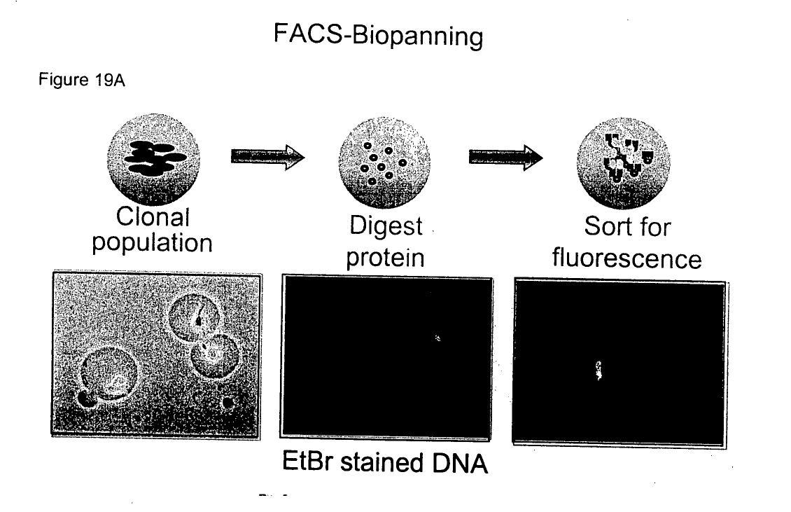

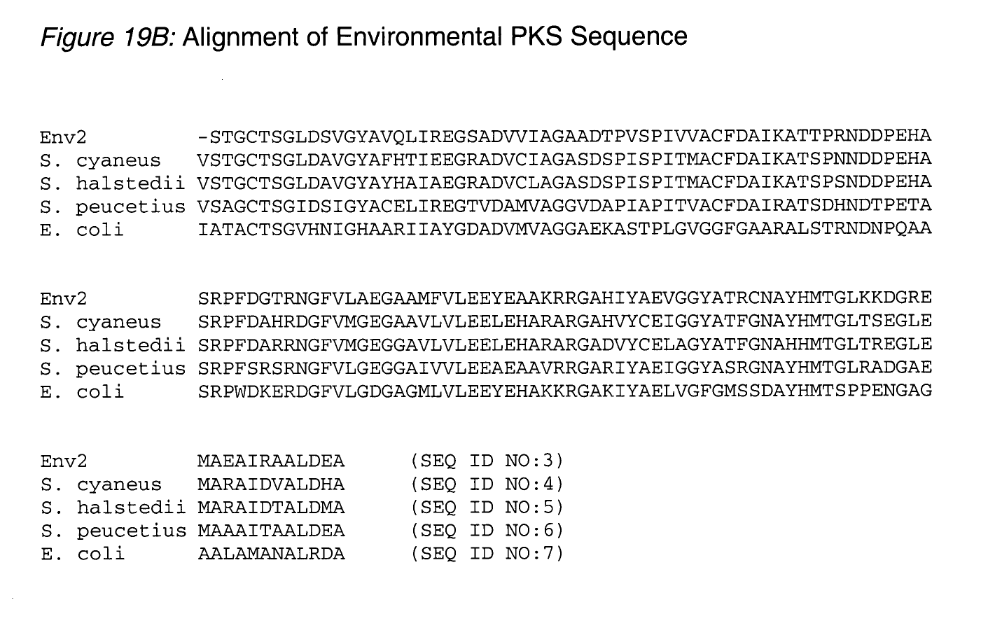

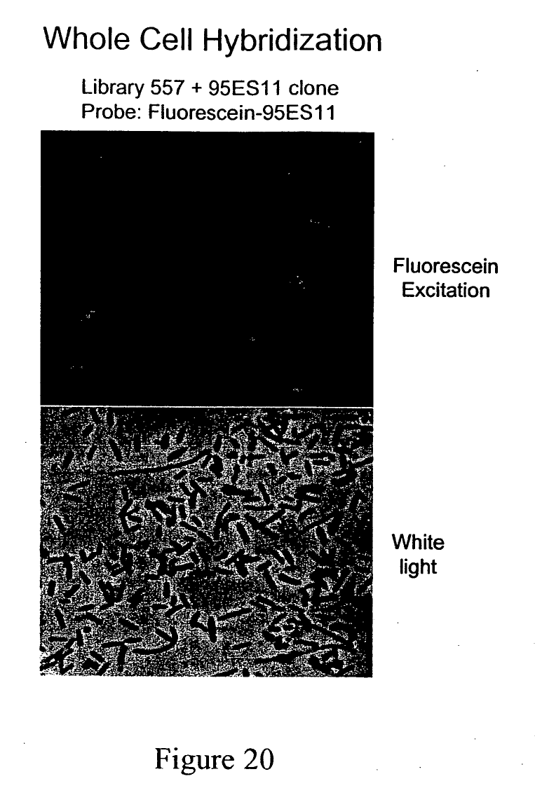

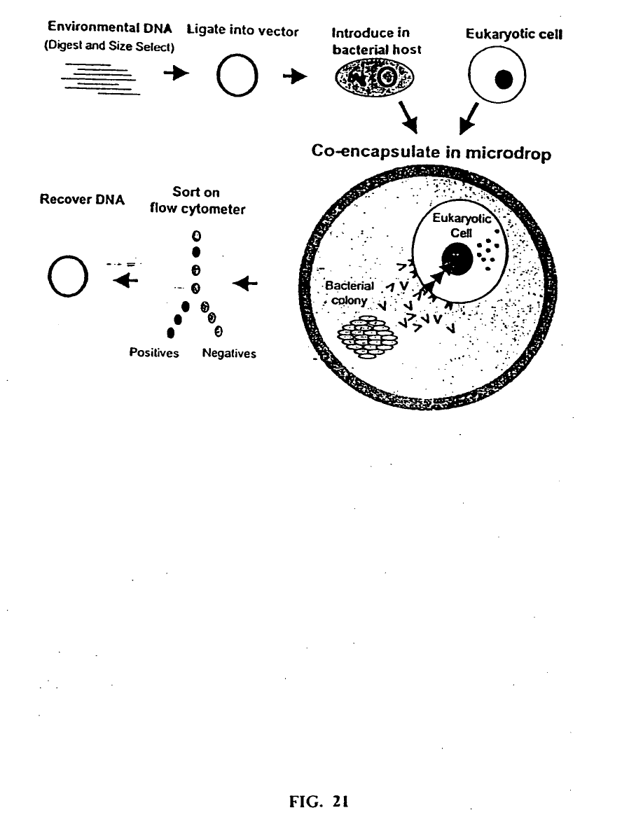

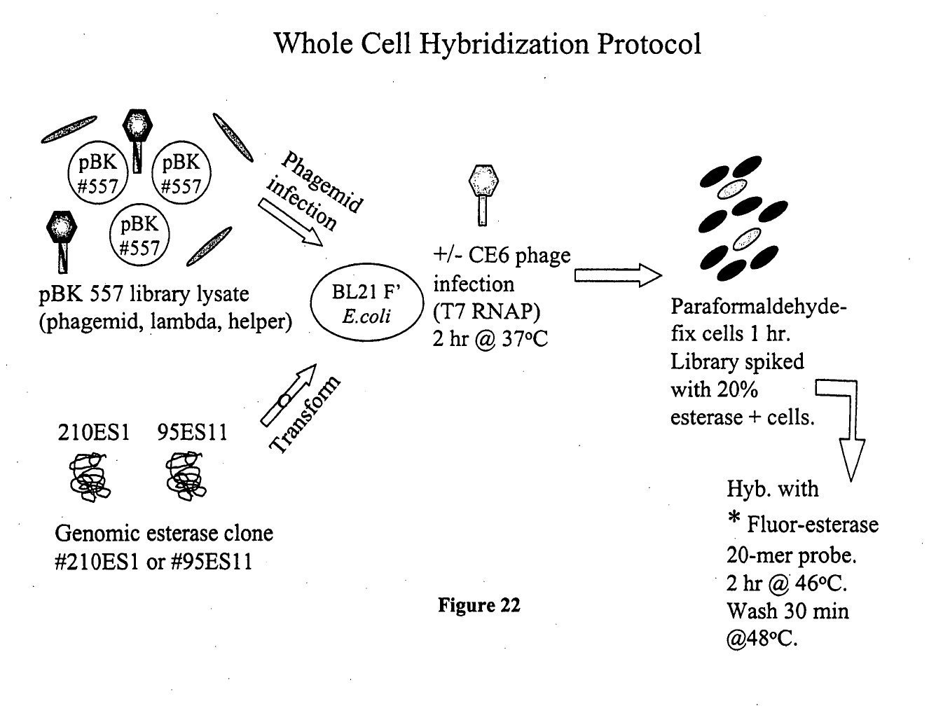

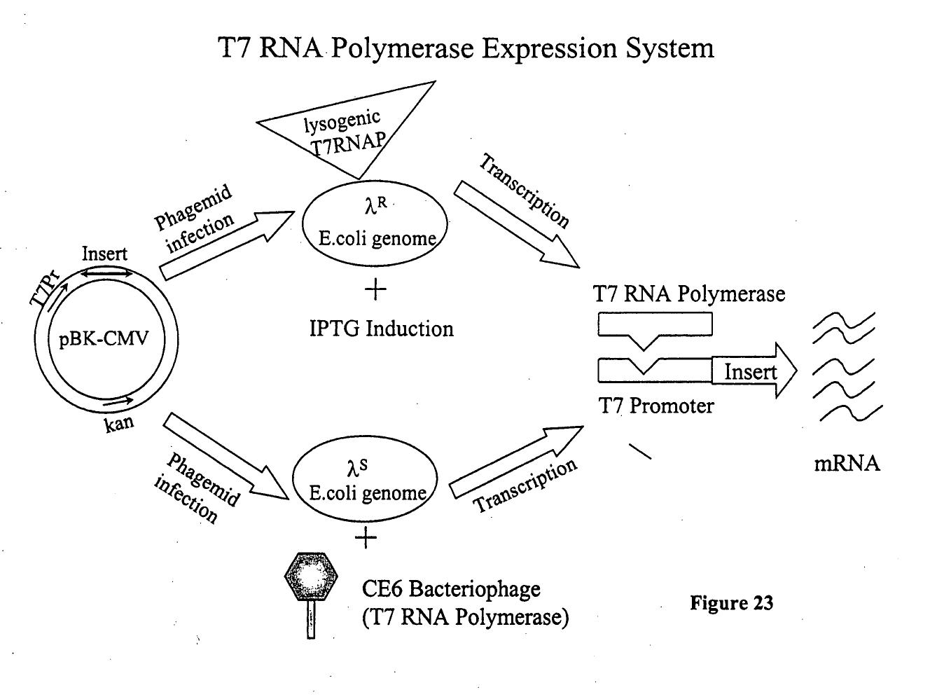

FIG. 5 shows an overview of the procedures used to construct an environmental library from a mixed picoplankton sample. A stable, large insert DNA library representing picoplankton genomic DNA was prepared as follows.

Cell collection and preparation of DNA. Agarose plugs containing concentrated picoplankton cells were prepared from samples collected on an oceanographic cruise from Newport, Oreg. to Honolulu, Hi. Seawater (30 liters) was collected in Niskin bottles, screened through 10 μm Nitex, and concentrated by hollow fiber filtration (Amicon DC10) through 30,000 MW cutoff polyfulfone filters. The concentrated bacterioplankton cells were collected on a 0.22 11m, 47 mm Durapore filter, and resuspended in 1 ml of 2×STE buffer (1M NaCl, 0.1M EDTA, 10 mM Tris, pH 8.0) to a final density of approximately 1×10¹⁰ cells per ml. The cell suspension was mixed with one volume of 1% molten Seaplaque LMP agarose (FMC) cooled to 40° C., and then immediately drawn into a 1 ml syringe. The syringe was sealed with parafilm and placed on ice for 10 min. The cell-containing agarose plug was extruded into 10 ml of Lysis Buffer (10 mM Tris pH 8.0, 50 mM NaCl, 0.1M EDTA, 1% Sarkosyl, 0.2% sodium deoxycholate, 1 mg/ml lysozyme) and incubated at 37° C. for one hour. The agarose plug was then transferred to 40 ml of ESP Buffer (1% Sarkosyl, 1 mg/ml proteinase K, in 0.5M EDTA), and incubated at 55° C. for 16 hours. The solution was decanted and replaced with fresh ESP Buffer, and incubated at 55° C. for an additional hour. The agarose plugs were then placed in 50 mM EDTA and stored at 4° C. shipboard for the duration of the oceanographic cruise.

One slice of an agarose plug (72 μl) prepared from a sample collected off the Oregon coast was dialyzed overnight at 4° C. against 1 ml of buffer A (100 mM NaCI, 10 mM Bis Tris Propane-HCl, 100 μg/ml acetylated BSA: pH 7.0 (@ 25° C.) in a 2 ml microcentrifuge tube. The solution was replaced with 250 μl of fresh buffer A containing 10 mM MgCl₂ and 1 mM DTT and incubated on a rocking platform for 1 hr at room temperature. The solution was then changed to 250 μl of the same buffer containing 4 U of Sau3A1 (NEB), equilibrated to 37° C. in a water bath, and then incubated on a rocking platform in a 37° C. incubator for 45 min. The plug was transferred to a 1.5 ml microcentrifuge tube and incubated at 68° C. for 30 min to inactivate the protein, e.g. enzyme, and to melt the agarose. The agarose was digested and the DNA dephosphorylased using Gelase and HK-phosphatase (Epicentre), respectively, according to the manufacturer's recommendations. Protein was removed by gentle phenol/chloroform extraction and the DNA was ethanol precipitated, pelleted, and then washed with 70% ethanol. This partially digested DNA was resuspended in sterile H₂O to a concentration of 2.5 ng/μl for ligation to the pFOS1 vector.

PCR amplification results from several of the agarose plugs (data not shown) indicated the presence of significant amounts of archaeal DNA. Quantitative hybridization experiments using rRNA extracted from one sample, collected at 200 m of depth off the Oregon Coast, indicated that planktonic archaea in (this assemblage comprised approximately 4.7% of the total picoplankton biomass (this sample corresponds to “PACI”-200 m in Table 1 of DeLong et al., high abundance of Archaea in Antarctic marine picoplankton, Nature, 371:695-698, 1994). Results from archaeal-biased rDNA PCR amplification performed on agarose plug lysates confirmed the presence of relatively large amounts of archaeal DNA in this sample. Agarose plugs prepared from this picoplankton sample were chosen for subsequent fosmid library preparation. Each 1 ml agarose plug from this site contained approximately 7.5×10⁵ cells, therefore approximately 5.4×10⁵ cells were present in the 72 μl slice used in the preparation of the partially digested DNA.

Vector arms were prepared from pFOS1 as described (Kim et al., Stable propagation of casmid sized human DNA inserts in an f-factor based vector, Nucl. Acids Res., 20:10832-10835, 1992). Briefly, the plasmid was completely digested with AstII, dephosphorylated with HK phosphatase, and then digested with BamHI to generate two arms, each of which contained a cos site in the proper orientation for cloning and packaging ligated DNA between 35-45 kbp. The partially digested picoplankton DNA, isolated by partial fragment gel electrophoresis (PFGE), was ligated overnight to the PFOS1 arms in a 15 μl ligation reaction containing 25 ng each of vector and insert and 1 U of T4 DNA ligase (Boehringer-Mannheim). The ligated DNA in four microliters of this reaction was in vitro packaged using the Gigapack XL packaging system (Stratagene), the fosmid particles transfected to E. coli strain DH10B (BRL), and the cells spread onto LB_{cm15} plates. The resultant fosmid clones were picked into 96-well microliter dishes containing LB_{cm15} supplemented with 7% glycerol. Recombinant fosmids, each containing cat 40 kb of picoplankton DNA insert, yielded a library of 3,552 fosmid clones, containing approximately 1.4×10⁸ base pairs of cloned DNA. All of the clones examined contained inserts ranging from 38 to 42 kbp. This library was stored frozen at −80° C. for later analysis.

Numerous modifications and variations of the present invention are possible in light of the above teachings; therefore, within the scope of the claims, the invention may be practiced other than as particularly described.

### EXAMPLE 4

### CsCl-Bisbenzimide Gradients

Gradient Visualization by UV:

Visualize gradient by using the UV handlamp in the dark room and mark bandings of the standard which will show the upper and lower limit of GC-contents.

Harvesting of the Gradients:

1. Connect Pharmacia-pump LKB P1 with fraction collector (BIO-RAD model 2128).
2. Set program: rack 3, 5 drops (about 100 ul), all samples.
3. Use 3 microtiter-dishes (Costar, 96 well cell culture cluster).
4. Push yellow needle into bottom of the centrifuge tube.
5. Start program and collect gradient. Don't collect first and last 1-2 ml depending on where your markers are.
   Dialysis
1. Follow microdialyzer instruction manual and use Spectra/Por CE Membrane MWCO 25,000 (wash membrane with ddH20 before usage).
2. Transfer samples from the microtiter dish into microdialyzer (Spectra/Por,
3. MicroDialyzer) with multipipette. (Fill dialyzer completely with TE, get rid of any air bubble, transfer samples very fast to avoid new air-bubbles).
4. Dialyze against TE for 1 hr on a plate stirrer.

### DNA Estimation with PICOGREEN™

1. Transfer samples (volume after dialysis should be increased 1.5-2 times) with multipipette back into microtiter dish.
2. Transfer 100 ul of the sample into Polytektronix plates.
3. Add 100 ul Picogreen-solution (5 ul Picogreen-stock-solution+995 ul TE buffer) to each sample.
4. Use WPR-plate-reader.
5. Estimate DNA concentration.

### EXAMPLE 5

### Bis-Benzimide Separation of Genomic DNA

A sample composed of genomic DNA from Clostridium perfringens (27% G+C), Escherichia coli (49% WC) and Micrococcus lysodictium (72% G+C) was purified on a cesium-chloride gradient. The cesium chloride (Rf=1.3980) solution was filtered through a 0.2 m filter and 15 ml were loaded into a 35 ml OptiSeal tube (Beckman). The DNA was added and thoroughly mixed. Ten micrograms of bis-benzimide (Sigma; Hoechst 33258) were added and mixed thoroughly. The tube was then filled with the filtered cesium chloride solution and spun in a VTi5O rotor in a Beckman L8-70 Ultracentrifuge at 33,000 rpm for 72 hours. Following centrifugation, a syringe pump and fractionator (Brandel Model 186) were used to drive the gradient through an ISCO UA-5 UV absorbance detector set to 280 nm. Three peaks representing the DNA from the three organisms were obtained. PCR amplification of DNA encoding rRNA from a 10-fold dilution of the E. coli peak was performed with the following primers to amplify eubacterial sequences:

| | |
|---|---|
| Forward primer: (27F) | |
| 5-AGAGTTTGATCCTGGCTCAG-3 | (SEQ ID NO:1) |
| Reverse primer: (1492R) | |
| 5-GGTTACCTTGTTACGACTT-3 | (SEQ ID NO:2) |

### EXAMPLE 6

### FACS/Biopanning

Infection of library lysates into Exp503 E. coli strain. 25 ml LB+Tet culture of Exp503 were cultured overnight at 37 C. The next day the culture was centrifuged at 4000 rpm for 10 minutes and the supernatant decanted. 20 ml 10 mM MgSO₄ was added and the OD₆₀₀ checked. Dilute to OD 1.0.

In order to obtain a good representation of the library, at least 2-fold (and preferably 5-fold) of the library lysate titer was used. For example: Titer of library lysate is 2×106 cfu/ml. Need to plate at least 4×106 cfu. Can plate approx. 500,000 microcolonies/150 mm LB-Kan plate. Need 8 plates. Can plate 1 ml of reaction/plate- need 8 mls of cells+lysate.

2-fold (ex. 2 ml) of library lysate was mixed with appropriate amount (e.g., 6 ml) of OD 1.0 Exp503. The sample was incubated at 37oC for at least 1 hour. Plated 1 ml reaction on 150 mm LB-Kan plate×8 plates and incubated overnight at 30oC. Harvesting, induction, and fixing of library in Exp503 cells. Scrape all cells from plates into 20 ml LB using a rubber policeman. Dilute cells approx. 1:100 (200 ul cells/20 ml LB) and incubate at 37oC until culture is OD 0.3. Add 1:50 dilution of 20% sterile Glucose and incubate at 37oC until culture is OD 1.0. Add 1:100 dilution of 1M MgSO4. Transfer 5 ml of culture to a fresh tube and the remaining culture can be used as an uninduced control if desired or discarded. Add MOI 5 of CE6 bacteriophage to the remaining 5 ml of culture. (CE6 codes for T7 RNA Polymerase) (e.g., OD 1=8×108 cells/ml×5 ml=4×109 cells×MOI 5=2×1010 bacteriophage needed). Incubate culture+CE6 for 2 hr at 37oC. Cool on ice and centrifuge cells at 4000 rpm for 10 min. Wash with 10 ml PBS. Fix cells in 600 ul PBS+1.8 ml fresh, filtered 4% paraformaldehyde. Incubate on ice for 2 hrs. (4% Paraformaldehyde: Heat 8.25 ml PBS in flask at 65oC. Add 100 ul 1M NaOH and 0.5 g paraformaldehyde (stored at 4oC.) Mix until dissolved. Add 4.15 ml PBS. Cool to 0oC. Adjust pH to 7.2 with 0.5 M NaH2PO4. Cool to 0oC. Syringe filter. Use within 24 hrs). After fixing, centrifuge at 4000 rpm for 10 min. Resuspend in 1.8 ml PBS and 200 ul 0.1% NP40. Store at 4oC overnight.

Hybridization of fixed cells. Centrifuge fixed cells at 4000 rpm for 10 min. Resuspend in 1 ml 40 mM Tris pH7.6/0.2% NP40. Transfer 100 ul fixed cells to an Eppendorf tube. Centrifuge for 1 min and remove supernatant. Resuspend each reaction in 50 ul Hybridization buffer (0.9 M NaCl; 20 mM Tris pH7.4; 0.01% SDS; 25% formamide—can be made in advance and stored at −20oC.). Add 0.5 nmol fluorescein-labeled primer to the appropriate reactions. Incubate with rocking at 46oC for 2 hr. (Hybridization temperature may depend on sequence of primer and template.) Add 1 ml wash buffer to each reaction, rinse briefly and centrifuge for 1 min. Discard supernatant. (Wash buffer: 0.9 M NaCl; 20 mM Tris pH 7.4; 0.01% SDS). Add another 1 ml of wash buffer to each reaction, and incubate at 48oC with rocking for 30 min. Centrifuge and remove supernatant. Visualize cells under microscope using WIB filter.

FACS sorting. Dilute cells in 1 ml PBS. If cells are clumping, sonicate for 20 seconds at 1.5 power. FAC sort the most highly fluorescent single-cells and collect in 0.5 ml PCR strip tubes (approximately one 96-well plate/library). PCR single-cells with vector specific primers to amplify the insert in each cell. Electrophorese all samples on an agarose gel and select samples with single inserts. These can be re-amplified with Biotin-labeled primers, hybridized to insert-specific primers, and examined in an ELISA assay. Positive clones can then be sequenced. Alternatively, the selected samples can be re-amplified with various combinations of insert-specific primers, or sequenced directly.

### EXAMPLE 7

### Large Insert FACS Biopanning Protocol

1. Encapsulate 1 vial of 3% home-made SeaPlaque gel. Each vial of gel can make 10⁶ GMD. Take 100 ul melt frozen fosmid pMF21/DH10B library, OD600=0.4 to encapsulate, centrifuge down to 10 ul. Melt agarose gel, add 100 ul FBS (fetal bovine serum) and vortex. Place in 50 C water in a beaker. Add 10 ul culture, vortex and add to 17 ml mineral oil. Shake for about 30 times, place on the One Cell machine. Blend at 2600 rpm 1 min at room temperature and 2600 rpm 9 minutes on ice. Wash with PBS twice. Resuspend in 10 ml LB+Apr⁵⁰, shake at 37° C. for 4 hours at 230 rpm. Check microscopically to see the growth and size of microcolonies.
2. Centrifuge at 1500 rpm for 6 min. GMDs are resuspend in 5 ml of 2×SSC and can be saved at 4° C. for several days. Take 200 ul GMD in 2×SSC for each reaction.
3. Resuspend in 10 ml 2×SSC/5% SDS. Incubate 10 min at RT shaking or rotating. Centrifuge.
4. Resuspend in 5 ml lysis solution containing proteinase K. Incubate 30 min at 37° C. shaking or rotating. Centrifuge.

Lysis Solution:

| | |
|---|---|
| 50 mM Tris pH8 | 1.5 ml 1M Tris |
| 50 mM EDTA | 1.5 ml 0.5M EDTA |
| 100 mM NaCl | 300 ul 5M NaCl |
| 1% Sarkosyl | 0.75 ml 20% Sarkosyl |
| 250 ug/ml Proteinase K | 375 ul proteinase K stock (10 mg/ml) |
| | 11.325 ml dH2O |

5. Resuspend in 5 ml denaturing solution. Incubate 30 min at RT shaking or rotating. Centrifuge at 1500 rpm for 5 min.

Denaturing Solution:

0.5M NaOH/1.5M NaCl
6. Resuspend in 5 ml neutralizing solution. Incubate 30 min at RT shaking or rotating. Centrifuge.

Neutralizing Solution:

0.5M Tris pH8/1.5M NaCl
7. Wash in 2×SSC briefly.
8. Aliquot 200 ul /R×N into microcentrifuge tubes, microcentrifuge and take out the 2×SSC. Add 130 ul “DIG EASY HYB” to prehyb for 45 minutes at 37° C. Do prehyb and hyb in Personal Hyb Oven.
9. Aliquot oligo probe and denature at 85° C. for 5 minutes, place on ice immediately. Add appropriate amount of probe (0.5-1 nmol/R×N) and return to rotating hyb. oven for O/N.
10. Prepare a 1% (10 mg/ml) solution of Blocking Reagent in PBS. Store at 4° C. for the day use.
11. Wash GMD's with 0.8 ml of 2×SSC/0.1% SDS RT 15 min, rotating. At the meantime, prewarm next wash solution.
12. Wash GMD's with 0.8 ml of 0.5×SSC/0.1% SDS 2×15 min at appropriate temp, rotating. If more stringency is required, the 2^{nd} wash can be done in 0.1×SSC/0.1% SDS.
13. Wash with 0.8 ml/R×N 2×SSC briefly.
14. Block the reaction w/130 ul 1% Blocking Reagent in PBS at RT for 30 minutes.
15. Add 1.4 ul anti-DIG-POD (so 1:100) and incubate at RT for 3 hours.
16. Wash GMDs w/0.8 ml PBS/RN 3×7 minutes at 37° C.
17. Prepare a tyramide working solution by diluting the tyramide stock solution 1:85 in Amplification buffer/0.0015% H₂O₂. Apply 130 ul tyramide working solution at RT and incubate in the dark at RT for 30 minutes.
18. Wash 3× for 7 min. in 0.8 ml PBS buffer @37° C.
19. Visualize by microscope and FACS sort.

### EXAMPLE 8

### Biopanning Protocol

Preparing Insert DNA from the Lambda DNA

PCR amplify inserts using vector specific primers CA98 and CA103.

CA98: ACTTCCGGCTCGTATATTGTGTGG
CA103: ACGACTCACTATAGGGCGAATTGGG

These primers match perfectly to lambda ZAP Express clones (pBKCMV).
Reagents: Lambda DNA prepared from the libraries to be panned (Librarians)
   Roche Expand Long Template PCR System #1-759-060
   Pharmacia dNTP mix #27-2094-01 or
   Roche PCR Nucleotide Mix (10 mM) #1-581-295 or
   Roche dNTP's—PCR grade #1-969-064
1. Make the insert amplification mix:
   X μl dH₂O (final 50 μl)
   5 μl 10× Expand Buffer #2 (22.5 mM MgCl₂)
   0.5 or 0.625 μl dNTP mix (20 mM each dNTP)
   10 ng (approx) lambda DNA per library (usually 1 μl or 1 μl 1:10 diln)
   1-2 μl CA98 (100 ng/μl or 15 μM)
   1-2 μl CA103 (100 ng/μl or 15 μM)
   0.5 μl Expand Long polymerase mix
2. PCR amplify:

Robocycler

| | | |
|---|---|---|
| 95° C. | 3 minute | x1 cycle |
| 95° C. | 1 minute | x30 cycles |
| 65° C. | 45 seconds | |
| 68° C. | 8 minute | |
| 68° C. | 8 minute | x1 cycle |
| 6° C. | | ∞ |

3. Analyze 5 μl of reaction product on a gel.
   Note: The reaction product should be a strong smear of products usually ranging from 0.5-5 kb in size and centered around 1.5-2 kb.
   Prepare Biotinylated Hook
Reagents: PCR reagents
   Biotin-14-dCTP (BRL #19518-018)
   Individual dNTP stock solutions (Roche dNTP's #1-969-064)
   Gene specific template and primers
   PCR purification kit (Roche #1732668 or Qiagen Qiaquick #28106)
1. Make 10× biotin dNTP mix:
   150 μl biotin-14-dCTP
   3 μl 100 mM dATP
   3 μl 100 mM dGTP
   3 μl 100 mM dTTP
   1.5 μl 100 mM dCTP
2. Make PCR mix:
   74 μl water
   10μl 10× Expand Buffer #1
   10 μl 10× biotin dNTP mix (step #1)
   2 μl Primer #1 (100 ng/μl)
   2 μl Primer #2 (100 ng/μl)
   1 μl template (gene specific) (100 ng/μl)
   1 μl Expand Long polymerase mix
3. PCR amplify:

Robocycler

| | | |
|---|---|---|
| 95° C. | 3 minute | x1 cycle |
| 95° C. | 45 seconds | x30 cycles |
| *° C. | 45 seconds | |
| 68° C. | ** minute | |
| 68° C. | 8 minute | x1 cycle |
| 6° C. | | ∞ |

*Use an annealing temperature appropriate for your primers.
** Allow 1 minute/kb of target length.

4. Cleanup the reaction product using a PCR purification kit. Elute in 50 μl 5T.1F or Qiagen's EB buffer (10 mM Tris pH 8.5).
5. Check 5 μl on an agarose gel.
   Note: The product may be slightly larger than expected due to the incorporated of biotin.
   Biopanning
Reagents: Streptavidin-conjugated paramagnetic beads (CPG MPG-Streptavidin 10 mg/ml #MSTR0502)(Dynal Dynabeads M-280 Streptavidin)
Sonicated, denatured salmon sperm DNA (heated to 95° C., 5 min) (Stratagene # 201190)
PCR reagents
dNTP mix
   Magnetic particle separator
   Topo-TA cloning kit with Top10F′ comp cells (Invitrogen #K4550-40)
   High Salt Buffer: 5M NaCl, 10 mM EDTA, 10 mM Tris pH 7.3
1. Make the following reaction mix for each library/hook combination:
   5 μg insert DNA (PCR amplified lambda DNA)
   100 ng Biotinylated hook (100 ng total if using more than one hook)
   4.5 μl 20×SSC for a 3× final concentration (or High Salt buffer)
   X μl dH₂O for a final volume of 30 μl
2. Denature by heating to 95° C. for 10 min. (Robocycler works well for this step).
3. Hybridize at 70° C. for 90 min. (Robocycler)
4. Prepare 100 μl of MPG beads for each sample:
   Wash 100 μl beads two times with 1 ml 3×SSC
   Resuspend in: 50 μl 3×SSC (or High Salt buffer)
      10 μl Sonicated, denatured salmon sperm DNA (10 mg/ml) to block (or 100 ng total)
      (Do not ice)
5. Add the hybridized DNA to the washed and blocked beads.
6. Incubate at room temp for 30 min, agitating gently in the hybridization oven.
7. Wash twice at room temp with 1 ml 0.1×SSC/0.1% SDS, (or high salt buffer) using magnetic particle separator.
8. Wash twice at 42° C. with 1 ml 0.1×SSC/0.1% SDS (or high salt buffer) for 10 min each. (magnet)
9. Wash once at room temp with 1 ml 3×SSC. (magnet)
10. Elute DNA by resuspending the beads in 50 μl dH₂O and heating the beads to 70° C. for 30 min or 85° C. for 10 min. in the hyb oven (or thermomixer at 500 rpm). Separate using magnet, and discard the beads.
11. PCR amplify 1-5 μl of the panned DNA using the same protocol as Preparing Insert DNA from the Lambda DNA above.
12. Check 5 μl on agarose gel.
   Note: The reaction product should be a strong smear of products usually ranging from 0.5-5 kb in size and centered around 1.5-2 kb.
13. Clone 1-4 μl into pCR2.1-TopoTA cloning vector.
14. Transform 2×3 μl into Top10F′ chemically comp cells. Plate each transformation on 2×150 mm LB-kan plates. Incubate at 30° C. overnight.
   (Ideal density is ˜3000 colonies per plate).
   Repeat transformation if necessary to get a representative number of colonies per library. Archive the Biopanned DNA.
15. Transfer plates to Hybridization group, along with appropriate templates and a single primer for run off PCR ³²P-labeling reactions.
   Analysis of Results
1. Filter lifts from plates will be performed, and hybridized to the appropriate probe. Resultant films will be given to the Biopanned.
2. Align films to original colony plates. Colonies corresponding to positive “dots-on-film” should be toothpicked, patched onto an LB-Kan plate, and inoculated in 4 ml TB-Kan. For automation, inoculate 1 ml TB-kan in a 96-well plate and incubate 18 hrs. at 37° C.
3. Overnight cultures are mini-prepped (Biomek if possible). Digest with EcoRI to determine insert size.
   2 μl DNA
   0.5 μl EcoRI
   1 μl 10× EcoRI buffer
   6.5 μl dH₂O
   Incubate at 37° C. for 1 hr. Check insert size on agarose gel.
Large insert clones (>500 bp) are then PCR confirmed if possible with gene specific primers.
4. Putative positive clones are then sequenced.
5. Glycerol stocks should be made of all interesting clones (>500 bp).

### EXAMPLE 9

### High Throughput Cultivation of Marine Microbes from Sea Sample

1. Preparation of Cell Suspension

Cells were obtained after filtering 110 L of surface water through a 0.22 μm membrane. The cell pellet was then resuspended with seawater and a volume of 100 μL was used for cell encapsulation. This provided cell numbers of approximately 10⁷ cells per mL.
2. Cell Encapsulation into GMDs

The following reagents were used: CelMix™ Emulsion Matrix and CelGel™ Encapsulation Matrix (One Cell Systems, Inc., Cambridge, Mass.), Pluronic F-68 solution and Dulbecco's Phosphate Buffered Saline (PBS, without Ca2+ and Mg2+). Scintillation vials each containing 15 ml of CelMix™ emulsion matrix were placed in a 40oC water bath and were equilibrated to 40oC for a minimum of 30 minutes. 30 ul of Pluronic Solution F-68 (10%) was added to each of 6 vials of melted CelGel™ agarose. The agarose mixture was incubated to 40oC for a minimum of 3 minutes. 100 ul of cells (resuspended in PBS) were added per 6 vials of the CelGel™ bottles and the resulting mixture was incubated at 40oC for 3 minutes. Using a 1 ml pipette and avoiding air bubbles, the CelGel™-cell mixture was added dropwise to the warmed CelMix™ in the scintillation vial. This mixture was then emulsified using the CellSys100™ MicroDrop maker as follows: 2200 rpm for 1 minute at room temperature (RT), then 2200 rpm for 1 minute on ice, then 1100 rpm for 6 minutes on ice, resulting in an encapsulation mixture comprised of microdrops that were approximately 10-20 microns in diameter. The encapsulation mixture was then divided into two 15 ml conical tubes and in each vial, the emulsion was overlayed with 5 ml of PBS. The vials tubes were then centrifuged at 1800 rpm in a bench top centrifuge for 10 minutes at RT, resulting in a visible Gel MicroDrop (GMD) pellet. The oil phase was then removed with a pipette and disposed of in an oil waste container. The remaining aqueous supernatant was aspirated and each pellet was resuspended in 2 ml of PBS. Each resuspended pellet was then overlayed with 10 ml of PBS. The GMD suspension was then centrifuged at 1500 rpm for 5 minutes at RT. Overlaying process is repeated and the GMD suspension is centrifuged again to remove all free-living bacteria. The supernatant was then removed and the pellet was resuspended in 1 ml of seawater. 10 ul of the GMD suspension was then examined under the microscope in order to check for uniform GMD size and containment of then encapsulated organism into the GMD. This protocol resulted in 1 to 4 cells encapsulated in each GMD.
3. Sorting of GMDs Containing Single Cells for Identification by 16S rRNA Gene Sequence

On the first day of cultivation we sorted occupied GMDs that contained one to 4 cells, although most had only single cells. The sorting was done in a Mo-Flo instrument (Cytomation) by staining the cells inside the GMDs with Syto9 and then selecting green fluorescence (from the stain) and side-scatter as parameters for sorting gates. The staining was necessary since the cells are much smaller than E. coli and therefore show very low light-scatter signals. The target GMDs were sorted into a 96-well plate containing a PCR mixture and ready to be amplified immediately after sorting. We used a Hotstart enzyme (Qiagen) such as no reaction would occur before boiling for 15 min and therefore allows to work at room temperature before amplification. Before starting the PCR it was necessary to radiate the PCR mixture with a Stratalinker (Stratagene) at full power for 14 min to cross-link any potential genomic DNA present in the mixture before sorting. The primers used include the pair 27F and 1392R and 27F and 1522R according to the positions in E. coli gene sequence. The primers were obtained from IDT-DNA Technologies and were purified by HPLC. The primer concentration used in the reactions was 0.2 μM. We used a “touchdown” program consisting of 3 stages: a) boiling 15 min, b) 15 cycles decreasing the annealing temperature from 62 to 55° C. by 0.5 degrees per cycle, c) a series of cycles (20-40) increasing the annealing time 1 sec per cycle starting with 30 sec but keeping the temperature constant at 55° C. All the other stages of the PCR were as recommended by manufacturer. This protocol allowed the amplification of the 16S rRNA gene from individual cells encapsulated or small consortia of cells. The PCR products were then cloned into TOPO-TA (Invitrogen) cloning vectors and sequenced by dye-termination cycle sequencing (Perkin-Elmer ABI).

Cell Growth of Encapsulated Cells Inside GMDs

The encapsulated GMDs were placed into chromatography columns that allowed the flow of culture media providing nutrients for growth and also washed out waste products from cells. The experiment consisted of 4 treatments including the use of seawater, and amendments (inorganic nutrients including trace metals and vitamins, amino acids including trace metals and vitamins, and diluted rich organic marine media). This different set of nutrients provided a gradient to bias different microbial populations. The seawater used as base for the media was filter sterilized through a 1000 kDa and a 0.22 μm filter membranes prior to amendment and introduction to the columns. The cells were then incubated for a period of 17 weeks and cell growth was monitored by phase contrast microscopy. Cell identification was done by 16S rRNA gene sequence of grown colonies.
4. Sorting of GMDs Containing Colonies Consisting of One or More Cell Types

To identify the diversity and the community composition of the different treatments we performed a “bulk sorting” of the GMDs. This was done by taking a subsample of the GMDs from each column and run them into the Flow-cytometer. We selected as gating criteria forward- and side-scatter as occupied GMDs with a colony of 10 or more cells of individual cell sizes ranging from 0.5 to 5 μm were easy to discriminate from empty GMDs. We verified each time by phase contrast microscopy that we selected the correct gate for sorting. We then sorted a total of 300 GMDs per each individual PCR reaction (prepared as above) and ran the reaction in a thermocycler for a total of 50 to 60 cycles to have enough PCR product to be visualized by gel electrophoresis. The resulting PCR reactions from the same column were combined (2 to 4 replicates), cloned and sequenced as above to assess the phylogenetic diversity from each column and observe the bias effect resulting from the use of different nutrient regimes.

Gene Sequencing and Phylogenetic Analyses

The gene sequences were aligned and compared to our 16S rRNA database with the ARB phylogenetic program. Maximum Parsimony and neighbor joining trees were constructed using the amplified gene sequences (approximately 1400 bp).

### EXAMPLE 10

### Microextraction Procedure

A single copy of Streptomyces containing clones from a mixed population are FACS-sorted onto agar, allowed to develop into individual colonies, and bioassayed as individual clones.

Construction of a Clone Expressing a Bioactive Metabolite

A genomic library of Streptomyces murayamaensis is constructed in pJO436 (Bierman et al., Gene 1991 116:43-49) vector and hybridized with probes for polyketide synthase. A clone (1B) which hybridized was chosen and shuttled into Streptomyces venezuelae ATCC 10712 strain. The vector pMF 17 was also introduced into S. diversa as a negative control. When bioassayed on solid media, clone 1B expressed strong bioactivity towards Micrococcus luteus demonstrating that the insert present in clone 1B encoded a bioactive polyketide molecule.

### EXAMPLE 11

### FACS-Sorting of S. venezuelae Clones

The S. venezuelae exconjugant spores containing clone 1B, as well as pJO436 vector, are FACS-sorted in 48-well, 96-well, and 384-well format into corresponding plates containing MYM agar+Apramycin 50 ug/ml. The single spore clones were allowed to germinate, grow and sporulate for 4-5 days.

Natural product extraction procedure: After the clones were fully grown and sporulated for 4-5 days, following volumes of solvent methanol were added to the each well containing the clones.
48 well format: 0.8 ml
96 well format: 0.100 ml
384 well format: 0.06 ml
   The plates were incubated at room temperature overnight.
   The next day, the following volumes were recovered from the wells containing the clones.
48 well format: 0.3 ml
96 well format: 0.060 ml
384 well format: 0.030 ml

The extracts were assayed from a single well, and after combining extracts from 2, 4 and 10 wells. The methanol extract was dried and resuspended in 40 ul of methanol:water and 20 ul of which was assayed against M. luteus as the indicator strain.

A single colony of S. venezuelae containing clone 1B produced enough bioactive molecule, in 48-well, 96-well as well as 384-well format, to be extracted by the microextraction procedure and to be detected by bioassay.

### EXAMPLE 12

### Expression of Actinorhodin Pathway in S. venezuelae 10712

When Sau3A pIJ2303 library constructed in pJO436 was introduced into S. venezuelae, one exconjugant which appeared blue-grey in color was spotted. This exconjugant showed blue pigment on R2-S agar demonstrating the successful expression of a heterologous pathway (actinorhodin) pathway in S. venezuelae. JO436

Segregational Stability of S. venezuelae 10712 (pJO436::Actinorhodin)

Since Streptomyces clones for small molecule production are grown in absence of antibiotic selection, it was important to determine how stable the S. venezuelae pJO436 recombinant clones are. The S. venezuelae 10712 (pJO436::actinorhodin) clone was used as an example.

The act clone was grown in R2-S liquid cultures with and without apramycin and total cell count was done by plating on R2-S agar with and without apramycin. The act clone gave 100% and 96% apramycin resistant colonies when grown with and without apramycin, respectively. This demonstrates that S. venezuelae pJO436 clones are quite stable segregationally.

Expression Stability of S. venezuelae 10712 (pJO436::Actinorhodin)

Expression of the actinorhodin gene cluster in S. venezuelae 10712 has been demonstrated. However, when this clone was grown in liquid cultures it failed to produce actinorhodin, as determined by the absence of its blue color. Nonetheless, when mycelia from such cultures were plated on solid media, actinorhodin producing colonies were clearly evident. The majority of the colonies produced a faint blue color while a few colonies produced abundant actinorhodin. These colonies which produce actinorhodin abundantly have been named as HBC (hyper blue clones) clones.

These observations demonstrate that perhaps in HBC clones, a host mutation has occurred which allows very efficient actinorhodin expression. Mutations which could lead to efficient actinorhodin expression could include a variety of targets such as, elimination of negative regulators like cutRS, overexpression of positive regulators, or efficient expression of pathways which provide precursors for actinorhodin. The hyper production of actinorhodin by the HBC clones thus strongly demonstrates that it is indeed possible for us to construct a strain which is more optimized for heterologous expression of small molecules, by random mutagenesis or by specific cutRS knockout mutagenesis.

Construction of a Jadomycin Blocked Mutant of S. venezuelae

Orf1 of the jadomycin biosynthetic gene cluster was chosen as a target. Primers were designed so as to amplify jad-L and jad-R fragments with proper restriction sites for future subcloning. S. venezuelae is reasonably sensitive to hygromycin and therefore, hygromycin resistance gene will be used to disrupt the orf-1 gene. The strategy used for disrupting the jadomycin orf-1 is described in the attached figure. The hyg-disrupted copy of the orf-1 gene will then be placed on pKC1218 and used for gene replacement in the S. venezuelae 10712, as well as VS153 chromosome.

Expression of the Yellow Clone in S. venezuelae

The single arm rescue technique to recover the yellow clone insert from S. lividans clone 525Sm575 was described. The recovered clone #3 was mated into S. venezuelae 10712 as well as VS153. Yellow color was evident after several days on both 10712 as well as VS153 plates but absent in the pJO436 vector alone controls. Three 10712 yellow clones were grown in liquid R2-S medium and all three produced yellow color profusely. This experiment has validated S. venezuelae as a host and pJO436 as the vector for heterologous expression for the second time, the first time being with the actinorhodin gene cluster. This yellow clone insert could now be used in validation of different strains in our strain improvement program.

Development of a Mating Protocol in a Microtiter Plate Format.

In order to have the individual E. coli donor clones archived, we are attempting to develop a mating protocol in a microtiter plate format. According to this protocol, we plan to sort the E. coli library into a 96-well microtiter plate. The matings with S. diversa would then be done in on a R2-S agar plate in an array format corresponding to the 96-well microtiter plate containing the E. coli clones. The bioassays can be either conducted on the mating R2-S plate or the clones can be first replica plated on to another suitable agar plate and then bioassayed. This approach will allow us to go back to the E. coli clones once we detect a bioactive clone among the S. diversa exconjugant library. The E. coli clone can then be mated back into S. diversa for re-transformation and confirmation of the bioactivity.

In a preliminary experiment, matings were done by spotting S. diversa spores together with E. coli donor cells on R2-S agar plate (rather than spreading). After about 8 hours the plate was overlayed as usual with apramycin and nalidixic acid. The exconjugants appeared only on those spots were E. coli donor was added, but not on those spots containing S. diversa spores alone. These initial data are very promising, although some more standardization needs to be done to develop this technique fully.

### EXAMPLE 13

### Production of Single Cells or Fragmented Mycelia

In order to produce single cells or fragmented mycelia, 25 ml MYM media was inoculated (see recipe below) in 250 ml baffled flask with 100 ul of Streptomyces 10712 spore suspension and incubated overnight at 30° C. 250 rpm. After a 24 hour incubation, 10 ml was transferred to 50 ml conical polypropylene centrifuge tube and centrifuged at 4,000 rpm for 10 minutes @ 25° C. Supernatant was decanted and the pellet was resuspended in 10 ml 0.05M TES buffer. The cells were sorted into MYM agar plates (sort 1 cell per drop, 5 cells per drop, 10 cells per drop) and we incubated the plates at 30° C.

MYM media (Stuttard, 1982, J. Gen. Microbiol. 128:115-121) contains: 4 g maltose, 10 g malt ext., 4 g yeast extract, 20 g agar, pH 7.3, water to 1 L.

### EXAMPLE 14

### An Exemplary Method for the Discovery of Novel Enzymes

The following describes a method for the discovery of novel enzymes requiring large substrates (e.g., cellulases, amylases, xylanases) using the ultra high throughput capacity of the flow cytometer. As these substrates are too large to get into a bacterial cell, a strategy other than single intracellular detection must be employed in order to use the flow cytometer. For this purpose, we have adapted the gel microdrop (GMD) technology (One Cell Systems, Inc.) Specifically, the enzyme substrate is captured within the GMD and the enzyme allowed to hydrolyze the substrate within this microenvironment. However, this method is not limited to any particular gel microdrop technology. Any microdrop-forming material that can be derivatized with a capture molecule can be used. The basic experimental design is as follows: Encapsulate individual bacteria containing DNA libraries within the GMDs and allow the bacteria to grow to a colony size containing hundreds to thousands of cells each. The GMDs are made with agarose derivatized with biotin, which is commercially available (One Cell Systems). After appropriate colony growth, streptavidin is added to serve as a bridge between a biotinylated substrate and the biotin-labeled agarose. Finally, the biotinylated substrate will be added to the GMD and captured within the GMD through the biotin-streptavidin-biotin bridge. The bacterial cells will be lysed and the enzyme released from the cells. The enzyme will catalyze the hydrolysis of the substrate, thereby increasing the fluorescence of the substrate within the GMD. The fluorescent substrate will be retained within GMD through the biotin-streptavidin-biotin bridge and thus, will allow isolation of the GMD based on fluorescence using the flow cytometer. The entire microdrop will be sorted and the DNA from the bacterial colony recovered using PCR techniques. This technique can be applied to the discovery of any enzyme that hydrolyzes a substrate with the result of an increased fluorescence. Examples include but are not limited to glycosidases, proteases, lipases, ferullic acid esterases, secondary amidases, and the like.

One system uses a biotin capture system to retain secreted antibodies within the GMD. The system is designed to isolate hybridomas that secrete high levels of a desired antibody. This basic design is to form a biotin-streptavidin-biotin sandwich using the biotinylated agarose, streptavidin, and a biotinylated capture antibody that recognizes the secreted antibody. The “captured” antibody is detected by a fluoresceinated reporter antibody. The flow cytometer is then used to isolate the microdrop based on increased fluorescence intensity. The potentially unique aspect to the method described here is the use of large fluorogenic substrates for the determination of enzyme activity within the GMD. Additionally, this example uses bacterial cells containing DNA libraries instead of eukaryotic cells and is not confined to secreted proteins as the bacterial cells will be lysed to allow access to the enzymes.

The fluorogenic substrates can be easily tailored to the particular enzyme of interest. Described below is a specific example of the chemical synthesis of an esterase substrate. Additionally, two examples are given which describe the different possible chemical combinations that can be used to make a wide variety of substrates.

### Example of Reaction Sequence Leading to GMD-Attachable Substrate

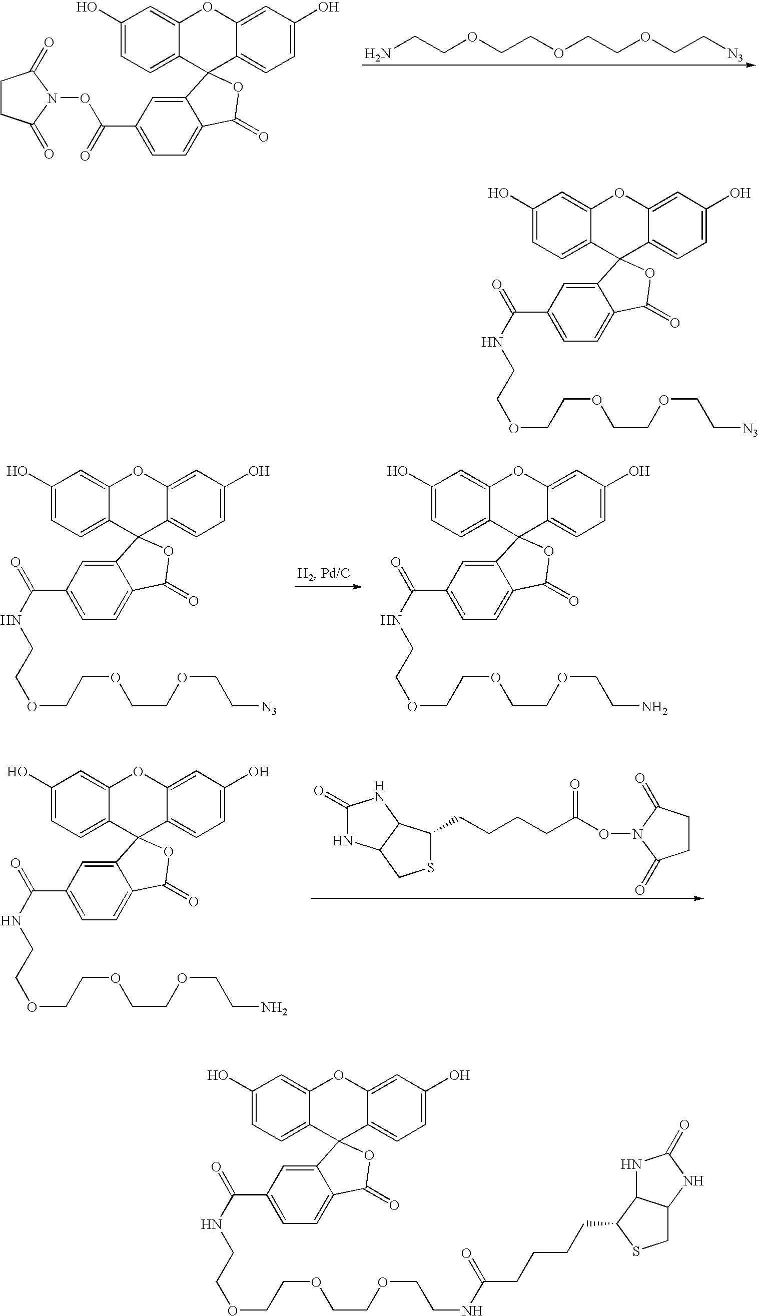

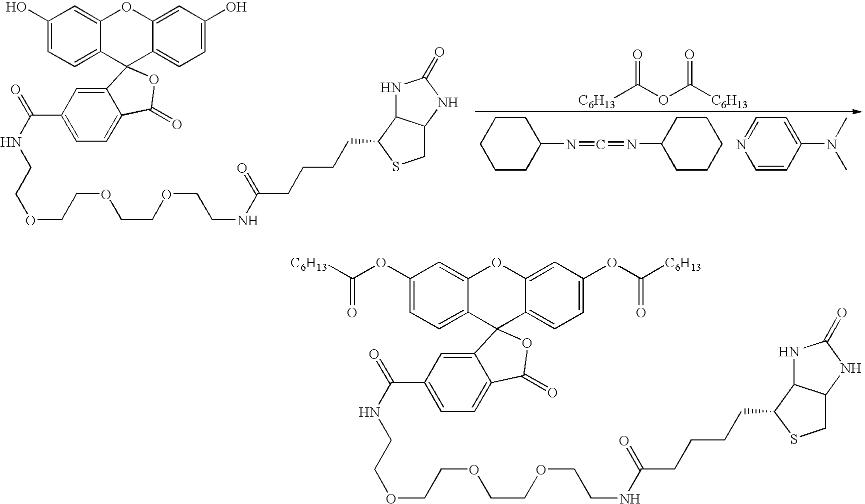

In the first step, 1-amino-11-azido-3,6,9-trioxaundecane [Reference 3], an asymmetric spacer, is attached to N-hydroxysuccinamide ester of 5-carboxyfluorescein (Molecular Probes). After reduction of the azide functional group on the end of the attached spacer (step 2), activated biotin (Molecular Probes) is attached to the amine terminus (step 3), and the sequence is completed by esterification of phenolic groups of the fluorescein moiety (step 4). The resulting compound can be used as a substrate in screens for esterase activity.
Design of GMD-Attachable Fluorogenic Substrates

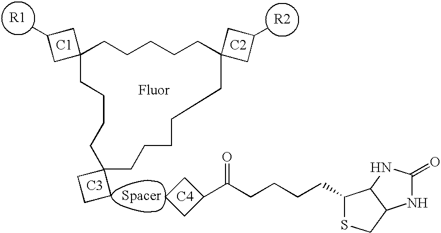

Fluor—core fluorophore structure, capable of forming fluorogenic derivatives, e.g. coumarins, resorufins, xanthenes, and others.

Spacer—a chemically inert moiety providing connection between biotin moiety and the fluorophore. Examples include alkanes and oligoethyleneglycols. The choice of the type and length of the spacer will affect synthetic routes to the desired products, physical properties of the products (such as solubility in various solvents), and the ability of biotin to bind to deep pockets in avidin.

C1, C2, C3, C4—connector units, providing covalent links between the core fluorophore structure and other moieties. C1 and C2 affect the specificity of the substrates towards different enzymes. C3 and C4 determine stability of the desired product and synthetic routes to it. Examples include ether, amine, amide, ester, urea, thiourea, and other moieties.

R1 and R2—functional groups, attachment of which provides for quenching of fluorescence of the fluorophore. These groups determine the specificity of substrates towards different enzymes. Examples include straight and branched alkanes, mono- and oligosaccharides, unsaturated hydrocarbons and aromatic groups.
Design of GMD-Attachable Fluorescence Resonance Energy Transfer Substrates

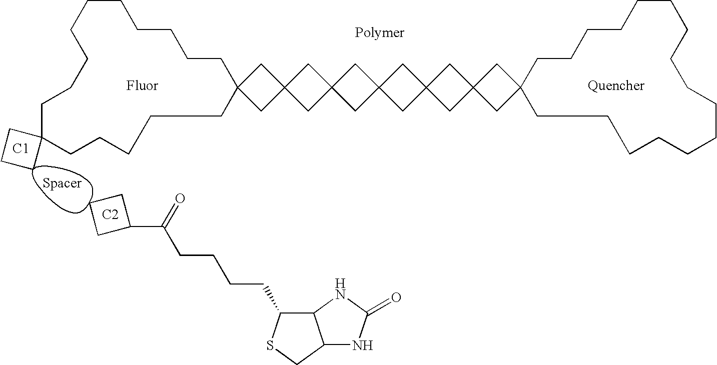

Fluor—A fluorophore. Examples include acridines, coumarins, fluorescein, rhodamine, BODIPY, resorufin, porphyrins, etc.

Quencher—A moiety, which is capable of quenching fluorescence of the fluorophore when located at a close enough distance. Quencher can be the same moiety as the fluorophore or a different one.

Polymer is a moiety, consisting of several blocks, a bond between which can be cleaved by an enzyme. Examples include amines, ethers, esters, amides, peptides, and oligosaccharides,

C1 and C2 are equivalent to C3 and C4 in the previous design.

Spacer is equivalent to Spacer in the previous design.

References:
[1] Gray, F, Kenney, J. S., Dunne, J. F. Secretion capture and report web: use of affinity derivatized agarose microdroplets for the selection of hybridoma cells. J Immunol. Meth. 1995, 182, 155-163.
[2] Powell, K. T. and Weaver, J. C. Gel microdroplets and flow cytometry: Rapid determination of antibody secretion by individual cells within a cell population. Bio/technology 1990, 8, 333-337.
[3] Schwabacher, A. W.; Lane, J. W.; Schiesher, M. W.; Leigh, K. M.; Johnson, C. W. J. Org. Chem. 1998, 63, 1727-1729.

### EXAMPLE 15

### An Exemplary Ultra High Throughput Screen: a Recombinant Approach

This example demonstrates an ultra high throughput screen for the discovery of novel anticancer agents. This method uses a recombinant approach to the discovery of bioactive molecules. The examples use complex DNA libraries from a mixed population of uncultured microorganisms that provide a vast source of natural products through recombinant expression from whole gene pathways. The two objectives of this Example include:
1) Engineering of mammalian cell lines as reporter cells for cancer targets to be used in ultra-high throughput assay system.
2) Detection of novel anticancer agents using an ultra high throughput FACS-based screening format.

The present invention provides a new paradigm for screening technologies that brings the small molecule libraries and target together in a three dimensional ultra high throughput screen using the flow cytometer. In this format, it is possible to achieve screening rates of up to 10⁸ per day. The feasibility of this system is tested using assays focused on the discovery of novel anti-cancer agents in the areas of signal transduction and apoptosis. Development of a validated assay should have a profound impact on the rate of discovery of novel lead compounds.

Experimental Design and Methods

1. Development of Cell Lines

The goal of this example is to develop an ultra high throughput screening format that can be used to discover novel chemotherapeutic agents active against a range of molecular targets known to be important in cancers. The feasibility of this approach will be tested using mammalian cell lines that respond to activation of the epidermal growth factor receptor (EGFR) with induction of expression of a reporter protein. The EGFR-responsive cells will be brought together with our microbial expression host within a microdrop (see Example 13 and co-pending U.S. Pat. No. 6,280,926, and U.S. application Ser. No. 09/894,956, both herein incorporated by reference). These expression hosts will be Streptomyces or E coli and will contain libraries derived from a mixed population of organisms, i.e. high molecular weight environmental DNA (10-100 kb fragments) cloned into the appropriate vectors and transferred to the host. These large DNA fragments will contain biosynthetic operons which consist of the genes necessary to produce a bioactive small molecule. A bioactive molecule from the microbial host will elicit a biological response in the mammalian cell which will induce expression of a fluorescent reporter. The entire microdrop will be individually sorted on the flow cytometer based on fluorescence and the DNA from the host recovered. The mixed population libraries may contain from 10⁴-10¹⁰ clones, including 10⁵, 10⁶, 10⁷, 10⁸, 10⁹, or any multiple thereof.

An assay based on the EGF receptor was chosen because of its possible role in the pathogenesis of several human cancers. The EGF-mediated signal transduction pathway is very well characterized and several inhibitors of the EGF receptor have been found from natural sources (21,22). The EGFR is one of the early oncogenes discovered (erbB) from the avian erythroblastosis retrovirus and due to a deletion of nearly all of the extracellular domain, is constitutively active (23). Similar types of mutations have been found in 20-30% of cases of glioblastoma multiforme, a major human brain tumor (24). Overexpression of EGFR correlates with a poor prognosis in bladder cancer (25), breast cancer (26,27), and glioblastoma multiforme (28). Most of these cancers occur in an EGF-secreting background and demonstrates an autocrine growth mechanism in these cancers. Additionally, EGFR is over-expressed in 40-80% of non-small cell lung cancers and EGF is overexpressed in half of primary lung cancers, with patient prognosis significantly reduced in cases with concurrent expression of EGFR and EGF (29,30). For these reasons, inhibitors of the EGF receptor are potentially useful as chemotherapeutic agents for the treatment of these cancers.

The goal of this experiment is to create mammalian cell lines that serve as reporter cells for anticancer agents. HeLa cells endogenously express the EGFR as confirmed by FACS analysis using the anti-EGFR antibody, Ab-1 (Calbiochem). In contrast, CHO cells have little or no expression of the EGFR. The gene encoding EGFR was obtained from Dr. Gordon Gill (University of California, San Diego) and cloned it into the pcDNA3/hygro vector. The resulting vector was transfected into CHO cells and stable transformants selected with hygromycin. Enrichment of high EGFR-expressing CHO cells was performed through two rounds of FACS sorting using the anti-EGFR antibody. For detection of the activated pathway, a parallel approach is being taken utilizing both the PathDetect system from Stratagene (San Diego, Calif.) and the Mercury Profiling system from Clontech (San Diego, Calif.). The Path Detect system has been validated by researchers as a means of detecting mitogenic stimuli (31,32).

The EGFR is a tyrosine kinase receptor that functions through the MAP-kinase pathway to activate the transcription factor Elk-1 (33). The PathDetect product includes a fusion trans-activator plasmid (pFA-Elk1) that encodes for expression of a fusion protein containing the activation domain of the Elk-1 transcription activator and the DNA binding domain of the yeast GAL4. A second plasmid contains a synthetic promoter with five tandem repeats of the yeast GAL4 binding sites that control expression of the Photinus pyralis luciferase gene. The luciferase gene was removed and replaced with the gene encoding for the destabilized version of the enhanced green fluorescent protein (EGFP) (plasmid designated pFR-d2EGFP). The two plasmids were transfected together into the EGFR/CHO and HeLa cells at a ratio of 10:1 (pFR-EGFP:pFA-Elk1) and stable transformants selected using the neomycin resistance gene located on the pFA-Elk1 plasmid. Thus, ligand binding to the EGFR will initiate a signal transduction cascade that results in activation of the Elk1 portion of the fusion protein, allowing the DNA binding domain of the yeast GAL4 to bind to its promoter and turn on expression of EGFP.

Stimulation in the presence of serum is not surprising as this signal transduction pathway is common to most growth factors and it is likely that many growth factors including EGF are present in the serum. After 24 hours of significant serum starvation, this response is greatly reduced (FIG. 2A). The next step will be to selectively stimulate these cells with recombinant EGF (Calbiochem) and isolate the highly responsive single clones using the flow cytometer. These clones will be selected by sorting simultaneously for high levels of GFP and the EGFR. The EGFR will be detected using an anti-EGFR antibody with a secondary antibody labeled with phycoerythrin. This system has the advantage that use of the yeast GAL4 promoter in these cells should keep background or spurious induction of EGFP to a minimum.

The second group of cell lines uses the Mercury Profiling system to assay the same EGFR pathway. This system responds to activation of the pathway with an increase in the expression of human placental secreted alkaline phosphatase (SEAP). A fluorescent signal will be obtained by the addition of the phosphatase substrate ELF-97-phosphate (Molecular Probes), which yields a bright fluorescent precipitate upon cleavage. The advantage of this approach over the PathDetect system is the ability to amplify the signal through enzyme catalysis for low-level activation of the pathway. This parallel approach will increase the probability of success in finding bioactive compounds. In the Mercury Profiling system, a vector containing the cis-acting enhancer element SRE and the TATA box from the thymidine kinase promoter is used to drive expression of alkaline phosphatase (pTA-SEAP). This system relies on the endogenous transactivators present in the cell, such as Elk-1, to bind the SRE element on the vector and drive expression of SEAP upon stimulation of EGFR. The pTA-SEAP vector was transfected into the EGFR/CHO and HeLa cells and stable transformants selected using neomycin. Again, stimulation of the pathway occurred in the presence of serum factors in the media. Upon serum starvation, this response was greatly reduced (FIG. 2B). Single high expressing clones will be isolated following stimulation with EGF and sorting using a flow cytometer.

Development of Ultra High Throughput FACS Assay

A complex mixed population libraries (>10⁶ primary clones/library) was generated that provided access to the untapped biodiversity that exist in the >99% uncultivable microorganisms. These novel libraries require the development of ultra high throughput screening methods to obtain complete coverage of the library. We propose developing an assay using the flow cytometer that allows detection of up to 10⁸ clones/day.

In this assay format (FIG. 1), an expression host (Streptomyces, E. coli) and a mammalian reporter cell will be co-encapsulated together within a microdrop. The microdrop holds the cells in close proximity to each other and provide a microenvironment that facilitates the exchange of biomolecules between the two cell types. The reporter cell will have a fluorescent readout and the entire microdrop will be run through the flow cytometer for clonal isolation. The DNA from the genes or pathway of interest will subsequently be recovered using in vitro molecular techniques. This assay format will be validated for the discovery of both EGFR inhibitors as well as for small molecules that induce apoptosis. With validation of this format, we will progress to the ultra high throughput screening phase designed to discover novel chemotherapeutic agents active against these important molecular mechanisms underlying tumorigenesis.

The feasibility of this approach will be analyzed initially using the engineered cell lines described above that respond to activation by EGF with increased expression of a reporter protein (i.e. EGFP or alkaline phosphatase). Additionally, this initial study will use an E. coli host that over-expresses human EGF as a secreted protein directed to the bacterial periplasm (34). This approach will allow us to validate the assay format prior to screening for inhibitors of the EGFR pathway using our E. coli and Streptomyces expression libraries. For this experiment, the engineered cell lines will be co-encapsulated together with the E. coli host at a ratio of one to one. The EGF-expressing bacteria will be allowed to grow and form a colony within the microdrop. Due to the vastly higher growth rate of bacteria, a colony of bacteria will form prior to any or minimal cell division of the eukaryotic cell. This colony will then provide a significantly increased concentration of the bioactive molecule. The bacterial colony will be selectively lysed using the antibiotic polymyxin at a concentration that allows cell survival (35). This antibiotic acts to perforate bacterial cell walls and should result in the release of EGF from these cells without affecting the eukaryotic cell. In the final discovery assays, this lysis treatment should not be necessary as the small molecule products will likely be able to freely diffuse out of the cell. The EGF will activate the signal transduction pathway in the eukaryotic cell and turn on expression of the reporter protein.

The microdrops will be run through the flow cytometer and those microdrops exhibiting an increased fluorescence will be sorted. The DNA from the sorted microdrops will be recovered using PCR amplification of the insert encoding for EGF. For the reporter cells expressing secreted alkaline phosphatase, a couple of additional steps are required to achieve a fluorescent readout. As the enzyme is secreted from the cell, it is possible to prevent the diffusion of the protein from the microdrop by selectively capturing it within the matrix of the microdrop. This can be accomplished by using microdrops made with agarose derivatized with biotin. By forming a sandwich with streptavidin and a biotinylated anti-alkaline phosphatase antibody, it is possible to capture alkaline phosphatase where it can catalyze the conversion of the ELF-97 phosphate substrate within the microdrop (FIG. 3A). This technique was successfully developed by One Cell Systems for the isolation of high expressing hybridomas (36, 37). In our hands, with the encapsulation of the SEAP expressing cells, we have shown that upon addition of the Elf-97 phosphatase substrate, a fluorescent precipitate forms within the microdrop (FIGS. 3B&C).

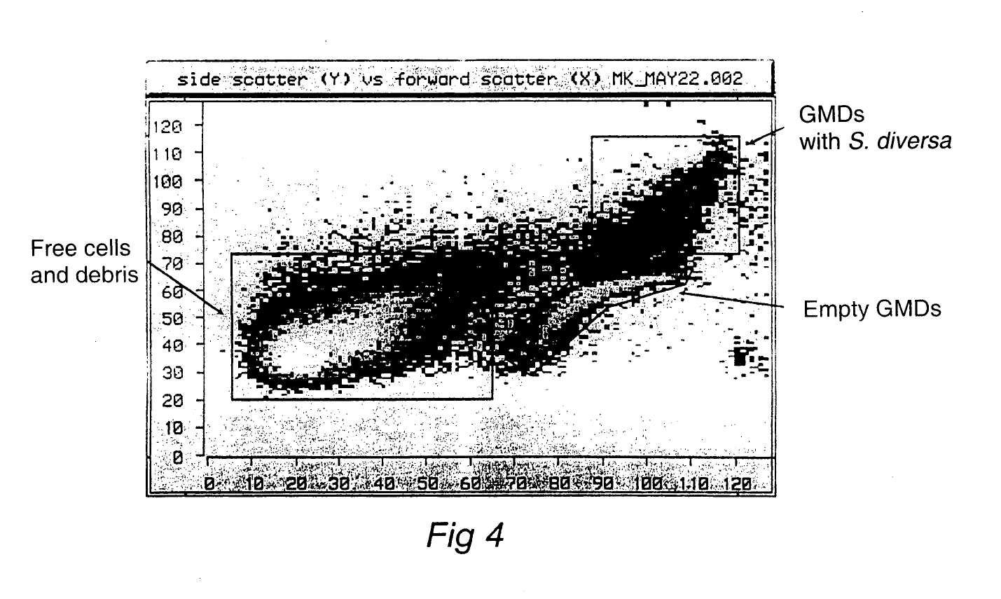

Initial experiments demonstrate the feasibility of co-encapsulating E. coli and mammalian cells (e.g., CHO) within microdrops. Microdrops were formed using 3% agarose dropped in oil and blended at 2600 rpm. The E. coli and CHO cells were encapsulated at a ratio of 1:1 (FIG. 4A). After 6 hours, the single bacterial cell grew into a colony containing thousands of cells (FIG. 4B). The cells within the microdrops were stained with propidium iodide to determine viability and approximately 70-85% of the CHO cells remained viable after 24 hours. Subsequent steps include determining the response of encapsulated clonal EGF-responsive mammalian cells to varying concentrations of EGF in the presence and absence of EGFR inhibitors such as Tyrphostin A46 or Tyrphostin A48 (Calbiochem). In addition, E. coli clones producing high levels of secreted EGF will be isolated using the Quantikine human EGF immunoassay (R&D Systems). Finally, these two cell types will be brought together within the microdrop and a change in fluorescence of the eukaryotic cell will be analyzed on the flow cytometer in the presence and absence of the EGFR inhibitors. A positive result in this experiment would be an increase in fluorescence that can be blocked by the EGFR inhibitors.

The next step will be to mix the EGF-expressing E. coli with non-expressing cells at varying ratios from 1:1,000 to 1:1,000,000 to mimic the conditions of an mixed population library discovery screen. The bacterial mixtures and the mammalian cells will be co-encapsulated as described above. The highly fluorescent microdrops will be individually sorted by the flow cytometer. To confirm a positive hit, the DNA will be recovered by PCR amplification using primers directed against the EGF gene. To improve the signal to noise ratio, it is likely that it will be necessary to undergo several rounds of enrichment before isolation of positive EGF-expressing clones, especially for the higher mixture ratios.

In this case, the microdrops will first be sorted in bulk, the microdrop material removed with GELase (Epicentre Technologies) and the bacteria allowed to grow. The encapsulation protocol will be repeated with fresh eukaryotic cells until a highly enriched population is observed. At this point, single microdrops will be isolated and recovery of the EGF-expressing clone confirmed by PCR. With validation of this assay, the goal will be to screen for inhibitors of the EGFR using our mixed population libraries expressed in optimized E. coli and Streptomyces hosts. This assay will be done in the presence of EGF and the assay endpoint will be a decrease in fluorescence. This format is not limited to only EGFR inhibitors as any protein within this pathway could be inhibited and would appear positive in this screen. Likewise, this screen can also be adapted to the multitude of anti-cancer targets that are known to regulate gene expression. In fact, using this present system, with the addition of the appropriate receptors, it would be possible to screen for inhibitors of other growth factors such as PDGF and VEGF.

If an increase in fluorescence is not observed with co-encapsulation of the EGF-expressing cells and the mammalian reporter cell, there could be several reasons. First, it is possible that the EGF diffuses out of the cell too quickly to elicit a response. In this case, it will be necessary to modify the microdrops to limit diffusion and concentrate the bioactive molecule at the site of the reporter cell. It is also possible that in the specific case of the EGF assay, the cells will not continue to produce EGF after polymyxin treatment and thus, the incubation time of the reporter cells with EGF will be minimal. This is unlikely as the polymyxin treatment used will be at concentrations well below that which produces decreased cell viability. However, if EGF is not continually expressed in this system, other permeabilization methods will be explored that do not significantly affect cell metabolism, such as the bacteriocin release protein (BRP) system (Display Systems Biotech). The BRP opens the inner and outer membranes of E. coli in a controlled manner enabling protein release into the culture medium. This system can be used for large-scale protein production in a continuous culture and thus should be compatible with cell survival.

Apoptosis, or programmed cell death, is the process by which the cell undergoes genetically determined death in a predictable and reproducible sequence. This process is associated with distinct morphological and biochemical changes that distinguish apoptosis from necrosis. The malfunctioning of this essential process can often lead to cancer by allowing cells to proliferate when they should either self-destruct or stop dividing. Thus, the mechanisms underlying apoptosis are currently under intense scrutiny from the research community and the search for agents that induce apoptosis is a very active area of discovery.

The present invention provides an assay for the discovery of apoptotic molecules using our ultra high throughput encapsulation technology. The source of these small molecules will come from our extremely complex mixed population libraries expressed in Streptomyces and E. coli host strains. These host strains will be co-encapsulated together with a eukaryotic reporter cell, the small molecule will be produced in the bacterial strain, and will act on the mammalian reporter cell which will respond by induction of apoptosis. Apoptosis will be detected using a fluorescent marker, the entire microdrop sorted using the flow cytometer, and the DNA of interest recovered. The feasibility of this assay will be determined using our optimized Streptomyces host strain, S. diversa, co-encapsulated with the apoptotic reporter cell derived from human T cell leukemia (e.g., Jurkat cells). The pathway controlling production of the anti-tumor antibiotic, bleomycin, will be cloned into S. diversa as the source of an apoptosis-inducing agent. The readout for induction of apoptosis in Jurkat cells will be obtained using the fluorescent marker, Alexis 488-annexin V™.

The bleomycin group of compounds are anti-tumor antibiotics that are currently being used clinically in the treatment of several types of tumors, notably squamous cell carcinomas and malignant lymphomas. However, widespread use of bleomycin congeners has been limited due to early drug resistance and the pulmonary toxicity that develops concurrent with administration of this drug. Thus, there is continuing effort to find novel small molecules with better clinical efficacy and lower toxicity. Bleomycin congeners are peptide/polyketide metabolites that function by binding to sequence selective regions of DNA and creating single and double stranded DNA breaks. Several in vitro and in vivo assays have shown that bleomycin induces apoptosis in eukaryotic cells (43-45). The biosynthetic gene cluster encoding for the production of bleomycin has recently been cloned from Streptomyces verticillus and is encoded on a contiguous 85 kb fragment (46). We propose to clone this pathway into a BAC vector to use as a source of apoptotic agents in eukaryotic cells. A library will be made from the S. verticillus ATCC15003 strain and cloned into the BAC vector, pBlumate2. As the sequence for this pathway is known, probes will be designed against sequences from the 5′ and 3′ ends of the pathway. The library will be introduced into E. coli and screened using colony hybridization with the probe generated against one end of the pathway. Positive clones will subsequently be screened with the second probe to identify which clone contains the entire pathway. Clones containing the complete pathway will be transferred into our optimized expression host S. diversa by mating. Expression of bleomycin will be detected using whole cell bioassays with Bacillus subtillis.

Jurkat cells are the classic human cell line used for studies of apoptosis. The fluorescent Alexis 488 conjugate of annexin V (Molecular Probes) will be used as the marker of apoptosis in these cells. Annexin V binds to phosphotidylserine molecules normally located on the internal portion of the membrane in healthy cells. During early apoptosis, this molecule flips to the outer leaf of the membrane and can be detected on the cell surface using fluorescent markers such as the annexin V-conjugates. The bleomycin-induced apoptotic response in Jurkat cells will initially be characterized by varying both the concentrations of the exogenously administered drug and the incubation time with the drug. Alexis 488-annexin V will then be add to the cells and the level of fluorescence analyzed on the flow cytometer. Necrotic cell death will be determined using propidium iodide and the apoptotic population will be normalized to this value.

Co-encapsulation of S. diversa with CHO cells within microdrops produced very similar results to the E. coli co-encapsulation. S. diversa grew well in the eukaryotic media and the CHO cell survival rate was high after 24 hours. In this experiment, the S. diverse clone expressing bleomycin will be co-encapsulated with the Jurkat cell line. S. diversa will be allowed to grow into a colony within the microdrop and begin production of bleomycin. The microdrops will be periodically analyzed over time for induction of apoptosis using the Alexis 488-annexin V conjugate on the microscope and flow cytometer. After noting the time for induction of apoptosis, a mixing experiment similar to that described for the EGF experiment will be performed. Bleomycin-expressing and non-expressing cells will be mixed together at ratios of 1:1000 to 1:1,000,000. Co-encapsulation of the mixtures with Jurkat cells will be performed and the appropriate incubation time maintained. These microdrops will then be stained with Alexis 488-annexin V and sorted on the flow cytometer. Confirmation of a positive bleomycin-expressing sorted clone will be performed by PCR amplification of a portion of the pathway. Again, it is likely that enrichment of these mixtures will be necessary using a few rounds of bulking sorting on the flow cytometer.

If no apoptosis is observed in the initial assay, confirmation of bleomycin production will be performed by sorting of the encapsulated S. diversa clone into 1536 well plates. After a predetermined incubation period, the supernatant will be removed and spotted on filter disks for whole cell bioassays using the susceptible strain B. subtilis. Use of the 1536 well plates will hopefully avoid significant dilution of the antibiotic in the media. As cloning of the bleomycin pathway is quite recent, it has not yet been heterologously expressed from the complete pathway. However, Du et al demonstrated the heterologous bioconversion of the inactive aglycones into active bleomycin congeners by cloning a portion of the pathway into a S. lividans host (46). If bleomycin expression is not detectable in our assay, we will employ a similar strategy using our host strain S. diversa. If little bleomycin production is detected under these conditions, it will be necessary to optimize the culture conditions for S. diversa to induce pathway expression within the microdrop. On the other hand, if bleomycin is produced but apoptosis is not observed, it is possible that the molecule is diffusing away from the microdrop too quickly and it will be necessary to optimize the microdrop technology to concentrate the metabolite at the site of the reporter cell.

Optimization of S. diversa Secondary Metabolite Expression in Microdrops

Induction of pathway expression is an issue that is not limited to the bleomycin example. Bioactive small molecules within microorganisms are often produced to increase the host's ability to survive and proliferate. These compounds are generally thought to be nonessential for growth of the organism and are synthesized with the aid of genes involved in intermediary metabolism, hence the name “secondary metabolites.” Thus, the pathways controlling expression of these secondary metabolites are often regulated under non-optimal conditions such as stress or nutrient limitation. As our system relies on use of the endogenous promoters and regulators, it might be necessary to optimize conditions for maximal pathway expression.

There are several methods that can used to optimize for increased pathway expression within the microdrops. For easy detection of maximal expression, we will construct a transposon containing a promoter-less GFP. The enhanced GFP optimized for eukaryotes will be used as it has a codon bias for high GC organisms. Transposition into a known pathway (e.g., actinorhodin) will be done in vitro and the vector containing the pathway purified. The transposants will be introduced into an E. coli host, screened for clones that express GFP, and positive clones isolated on the flow cytometer. With the transfer of the promoter-less gene for GFP into the pathway, increased fluorescence within the cells would demonstrate transcription of the pathway using the endogenous promoters located within the pathway. This clone will be used as a tool for quick detection of upregulation in pathway expression due to changes in the experimental conditions.

The S. diversa clone containing GFP and the actinorhodin pathway will be encapsulated in the microdrops and several different growth conditions will be tested, e.g., conditioned media, nutrient limiting media, known inducing factors, varying incubation times, etc. The microdrops will be analyzed under the microscope and on the flow cytometer to determine which conditions produce optimal expression of the pathway. These conditions will be verified for viability in eukaryotic cells as well. These optimized growth conditions will be confirmed using the bleomycin pathway to assess production of the secondary metabolite. Additionally, whole cell optimization of S. diversa is ongoing with production of strains that are missing different pleiotropic regulators that often negatively impact secondary metabolite production. As these strains are developed, they will be analyzed in the microdrops for enhanced pathway expression.

The proximity of the two cell types within the microdrop should result in a high concentration of the bioactive molecule at the site of the reporting cell. However, if rapid diffusion of the molecule from the microdrop prevents detection of the desired signal, it will be necessary to optimize the microdrop protocol or develop a new encapsulation technology. Concentration of the molecule at the site of the reporter cell could be achieved by a reduction in the microdrop pore size. Pore size reduction can be accomplished by one or a combination of the following approaches:

(i) “plugging” the holes with particles of an appropriate size, which are held in the pores by non-covalent or covalent interactions; (ii) cross-linking of the microdrop-forming polymer with low molecular weight agents; (iii) creation of an external shell around the microdrop with pores of smaller size than those in the current microdrop.
(i) Plugging the pores can be accomplished using polydisperse latexes with particles sized to fit within the pores of the microdrop. Latex particles may be modified on their surface such that they are attracted to the microdrop-forming polymer. For example, agarose-based microdrops carry a negative electrostatic charge on the surface. Thus, amidine-modified polystyrene latex particles (Interfacial Dynamics Corporation) will be attracted to the microdrop surface and the latex particles will effectively plug the microdrop pores provided that the charge density on the latex particles and the microdrop surface is high enough to sustain strong electrostatic bonds.
(ii) Cross-linking of agarose beads can be achieved by treating them with various reagents according to known procedures (47). For our purposes, the cross-linking needs to occur only on the surface of microdrop. Thus, it may be advantageous to use polymers carrying reactive groups for cross-linking of agarose, such that permeation of the cross-linking agent inside the microdrop is prevented.
(iii) Formation of classical (48) or polymerizable liposomes (49,50) around microdrops would provide a shell that could be an effective barrier even to small molecules. A wide variety of precursors for such liposomes as well as methods for their preparation have been reported (48-50) and most of them are applicable for our purposes. One of the possible limitations in choice of precursors stems from the intended use of microdrops for eventual screening by the flow cytometer. Thus, the liposomes should not absorb in the visible part of the spectrum.

It might also be necessary to use alternative methods and materials for preparation of the microdrops. Encapsulation of cells in polyacrylamide, alginate, fibrin, and other gel-forming polymers has been described (51). Another plausible candidate for encapsulation material is silica gel, which can be formed under physiological conditions with the assistance of enzymes (silicateins) (52) or enzyme mimetics (53). Additionally, various polymers may be used as the material for microdrop construction. Microdrops may be formed either upon polymerization of monomers (i.e. water-soluble acrylates or metacrylates) or upon gelation and/or cross-linking of preformed polymers (polyacrylates, polymetacrylates, polyvinyl alcohol). Since the formation of microdrops occurs simultaneously with encapsulation of living cells, such formation has to proceed under conditions compatible with cell survival. Thus, the precursors for microdrops (monomers or non-gelated polymers) should be soluble in aqueous media at physiological conditions and capable of the transformation into the microdrop material without any significant participation and/or emission of toxic compounds.

### EXAMPLE 16

### Identification of a Novel Bioactivity or Biomolecule of Interest by Mass Spectroscopic Screening

An integrated method for the high throughput identification of novel compounds derived from large insert libraries by Liquid Chromotography-Mass Spectrometry was performed as described below.

A library from a mixed population of organisms was prepared. An extract of the library was collected. Extracts from the libraries were either pooled or kept separate. Control extracts, without a bioactivity or biomolecule of interest were also prepared.

Rapid chromatography was used with each extract, or combination of extracts to aid the ionization of the compound in the spectra. Mass spectra were generated for the natural product expression host (e.g. S. venezuelae) and vector alone (e.g. pJO436) system. Mass spectra were also generated for the host cells containing the library extracts, alone or pooled. The spectra generated from multiple runs of either the background samples or the library samples were combined within each set to create a composite spectra. Composite spectra may be generated by using a percentage occurrence of an average intensity of each binned mass per time period or by using multiple aligned single mass spectra over a time period. By using a redundant sampling method where each sample was measured several times in the presence of other extracts, the novel signals that consistently occurred within a sample extract but not within the background spectra were determined.

The host-vector background spectrum was compared to the mass spectra obtained from large insert library clone extracts. Extra peaks observed in the large insert library clone extracts were considered as novel compounds and the cultures responsible for the extracts were selected for scale culture so the compound can be isolated and identified.

Novel Metabolite Identification by Mass Spectroscopic Screening.

In integrated method for the high throughput identification of novel compounds derived from large insert libraries by LC-MS is described below. Liquid chromatography-mass spectrometry is used to determine the background mass spectra of the natural product expression host (e.g. S. diversa DS10 or DS4) and vector alone (e.g. pmf17) system. This host-vector background spectrum is compared to the mass spectra obtained from large insert library clone extracts. Extra peaks observed in the large insert library clone extracts are considered as novel compounds and the cultures responsible for the extracts are selected for scale culture so the compound can be isolated and identified.

In order to create the background and sample spectra, rapid chromatography is used to aid the ionization of the compounds in the extract. The spectra generated from multiple runs of either the background samples or the library samples are combined within each set to create a composite spectra. Composite spectra may be generated by using a percentage occurrence of an average intensity of each binned mass per time period or by using multiple aligned single mass spectra over a time period. Using a redundant sampling method where by each sample is measured several times in the presence of other extracts the novel signals that consistently occur within a sample extract but not present in the background spectra can be determined. The purpose of this invention is to identify novel compounds produced by recombinant genes encoding biosynthetic pathways without relying on the compounds having bioactivity. This detection method is expected to be more universal than bioactivity for identifying novel compounds.

Currently there is a similar method of examining culture mixtures by LC-MS with long chromatographic times (30-60 min) to bring compounds to a fairly high level of purity. This method relies on molecular weight searches for de-replication of known compounds. This slow method would also work to identify novel compounds in S. diversa libraries however the throughput would be inadequate for the number of samples we need to screen. There are a pair of publications describing rapid direct infusion analysis of samples to identify fermentation conditions which improve the biosynthetic productivity of strains. This method does not identify specific compound, it just correlates greater, more complex production with different culture conditions.

Shown below are the following:
1. Chromatographic gradient and mass spec conditions
   HPLC and MS setting for Mass Spec Screening.TXT
2. Pooling of samples sheet
   Sampling Strategy.htm
3. Sample flow using average method
   Mass Spec Screening Flow chart.doc
4. Matlab code for original average background
   Mass Spec Screening Summary6 Matlab code.txt
5. Matlab code under development for new single aligned peaks background determination for more accurate data analysis.
   Mass Spec Screening 2nd Data Analysis Program.txt

The method is best practiced with a set of control extracts and sample extracts. Mixing of the compounds in pools prior to analysis and deconvolution of the mixed extract pools will provide high throughput while maintaining the ability to measure each extract several times.

A secondary screen may be required to eliminate false positives.

This method is more specific for identifying potential novel compounds by molecular ion than current methods. This method uses a different data analysis strategy than the de-replication methods for the identification of specific peaks for new compounds in extracts. Using the molecular ion as a signal to collect on this method may be coupled to mass based collection methods for the rapid isolation of compounds.

Related References:
“Rapid Method to Estimate the Presence of Secondary Metabolites in Microbial”, Higgs, R. E.; Zahn, et al., Appl. Environ. Microbiol. 67:371-376.
“Use of direct-infusion electrospray mass spectrometry to guide empirical development of improved conditions for expression of secondary metabolites from Actinomycetes”, Zahn, et al., Appl. Envron. Microbiol. 67:377-386.

“A general method for the de-replication of flavonoid glycosides utilizing high performance liquid chromatography mass spectrometric analysis.” Constant, et al., Phytochemical analysis, 1997, 8:176-180.

Method Information
Gradient column analysis of crude extracts by positive ion mode.
1100 Quaternary Pump 1

| | |
|---|---|
| Control | |
| Column Flow | 1.000 ml/min |
| Stoptime | 4.00 min |
| Posttime | Off |
| Solvents | |
| Solvent A | 98.0% (Water) |
| Solvent B | 0.0% (MeOH) |
| Solvent C | 2.0% (AcCN) |
| Solvent D | 0.0% (iPrOH) |
| PressureLimits | |
| Minimum Pressure | 0 bar |
| Maximum Pressure | 400 bar |
| Auxiliary | |
| Maximal Flow Ramp | 100.00 ml/min\hat{ }2 |
| Primary Channel | Auto |
| Compressibility | 100 * 10\hat{ }−6/bar |
| Minimal Stroke | Auto |
| Store Parameters | |
| Store Ratio A | Yes |
| Store Ratio B | Yes |
| Store Ratio C | Yes |
| Store Ratio D | Yes |
| Store Flow | Yes |
| Store Pressure | Yes |
| Agilent 1100 Contacts Option | |
| Contact 1 | Open |
| Contact 2 | Open |
| Contact 3 | Open |
| Contact 4 | Open |

Timetable

| Time | Solv. B | Solv. C | Solv. D | Flow | Pressure |
|---|---|---|---|---|---|
| 0.00 | 0.0 | 2.0 | 0.0 | 1.000 | |
| 0.01 | 0.0 | 2.0 | 0.0 | | |
| 0.30 | 0.0 | 95.0 | 0.0 | | |
| 1.50 | 0.0 | 95.0 | 0.0 | | |
| 1.60 | 0.0 | 2.0 | 0.0 | | |
| 4.00 | 0.0 | 2.0 | 0.0 | | |

Agilent 1100 Contacts Option Timetabl

Timetable is empty.

Agilent 1100 Diode Array Detector 1
Signals

| | | | |
|---|---|---|---|
| Signal | Store | Signal, Bw | Reference, Bw [nm] |

| | | | | |
|---|---|---|---|---|
| A: | Yes | 215 | 4 | 450 100 |
| B: | No | 254 | 4 | 450 100 |
| C: | No | 280 | 4 | 450 100 |
| D: | No | 250 | 16 | Off |
| E: | No | 280 | 16 | Off |

| | |
|---|---|
| Spectrum | |
| Store Spectra | Apex + Baselines |
| Range from | 190 nm |
| Range to | 600 nm |
| Range step | 2.00 nm |
| Threshold | 1.00 mAU |
| Time | |
| Stoptime | As pump |
| Posttime | Off |
| Required Lamps | |
| UV lamp required | Yes |
| Vis lamp required | Yes |
| Autobalance | |
| Prerun balancing | Yes |
| Postrun balancing | No |
| Margin for negative Absorbance | 100 mAU |
| Peakwidth | >0.1 min |
| Slit | 4 nm |
| Analog Outputs | |
| Zero offset ana. out. 1 | 5% |
| Zero offset ana. out. 2 | 5% |
| Attenuation ana. out. 1 | 1000 mAU |
| Attenuation ana. out. 2 | 1000 mAU |

Mass Spectrometer Detector

| | |
|---|---|
| General Information | |
| Use MSD | Enabled |
| Ionization Mode | APCI |
| Tune File | atunes.tun |
| StopTime | asPump |
| Time Filter | Enabled |
| Data Storage | Condensed |
| Peakwidth | 0.15 min |
| Scan Speed Override | Disabled |

Signals

| | |
|---|---|
| [Signal 1] | |
| Polarity | Positive |
| Fragmentor Ramp | Disabled |

Scan Parameters

| Time | Mass Range | | | Gain | | Step- |
|---|---|---|---|---|---|---|
| (min) | Low | High | Fragmentor | EMV | Threshold | size |
| 0.00 | 110.00 | 1500.00 | 70 | 1.0 | 500 | 0.15 |

| | |
|---|---|
| [Signal 2] | |
| Polarity | Positive |
| Fragmentor Ramp | Disabled |

Scan Parameters

| Time | Mass Range | | | Gain | | Step- |
|---|---|---|---|---|---|---|
| (min) | Low | High | Fragmentor | EMV | Threshold | size |
| 0.00 | 110.00 | 1500.00 | 110 | 1.0 | 500 | 0.15 |

[Signal 3]
Not Active
[Signal 4]
Not Active
Spray Chamber

[MSZones]

| | | |
|---|---|---|
| Gas Temp | 350 C. | maximum 350 C. |
| Vaporizer | 375 C. | maximum 500 C. |
| DryingGas | 3.0 l/min | maximum 13.0 l/min |
| Neb Pres | 60 psig | maximum 60 psig |
| VCap (Positive) | 3000 V | |
| VCap (Negative) | 3000 V | |
| Corona (Positive) | 4.0 μA | |
| Corona (Negative) | 15 μA | |

FIA Series

| | |
|---|---|
| FIA Series in this Method | Disabled |
| Time Setting | |
| Time between Injections | 1.00 min |

Agilent 1100 Column Thermostat 1

| | |
|---|---|
| Temperature settings | |
| Left temperature | 35.0° C. |
| Right temperature | Same as left |
| Enable analysis | When Temp. is within setpoint +/−0.8° C. |
| Store left temperature | Yes |
| Store right temperature | No |
| Time | |
| Stoptime | As pump |
| Posttime | Off |
| Column Switching Valve | Column 2 |
| Timetable is empty | |

During the process create a background file by looking for a certain percentage signal occurrence per mass unit. Use the Summary.m program to create this background spectra for use later in step 5 below.

| | | |
|---|---|---|
| 1 | Optional - Pool samples | Use attached pooling strategy |
| 2 | Measure Data | Use LC - MS to acquire data |
| 3 | Extract Data | Extract mass spectra into .csv file |
| | | format |
| 4 | Identify consistent signals in sample | Compare same sample runs to each |
| | deconvolute pools if sample | other, using Summary.m program, bin |
| | pooling in step 1 was used. | frequently/universally occurring signals |
| 5 | Determine Unique Peaks in Sample vs. | 1. Convert percent occurrence per |
| | Background | mass into a new sample spectra file. |
| | | 2. Use Massieve to deterermine |
| | | unique peaks in all voltages and |
| | | chromatographic fractions compared |
| | | to background |
| | | 3. Create ‘Unique Peaks’ file for |
| | | each voltage, chromatographic peak |
| | | comparison. |
| 6 | Eliminate extra peaks by taking advantage | Feed ‘Unique Peak’ file for each sample |
| | of multiple MS detection channels and | back into Summary.m program, keep |
| | chromatographic conditions. | peaks that show up in more then one |
| | | Mass spectrometer channel or |
| | | chromatographic peak. |
| 7 | | Short list of novel compound signals |

### EXAMPLE 17

### Plasmid DNA Transformation Protocol for Pseudomonas

Preparation of Electroporation Competent Cells

1 ml of overnight culture is inoculated into 100 ml LB, bacteria are incubated in the 30° C. shaker until OD 600 reading reaches 0.5-0.7. The bacteria are harvested by spinning @ 3000 rpm for 10 minutes at 4° C.

The resulting cell pellet is washed with 100 ml ice-cold ddH20, spun @ 3000 rpm for 10 minutes at 4° C. to collect the cells. The washing is repeated. The cells are then washed with 50 ml 10% ice-cold glycerol (in ddH20) once and collected by spinning @ 3000 rpm for 10 minutes at 4° C. The bacteria cell is resuspended into 2 ml ice-cold 10% glycerol (in ddH20) 50 ul or 100 ul is aliquotted into each of the tubes and stored at −80° C.

Electroporation

1 μl plasmid DNA is mixed with 50 μl competent cell and kept on ice for 5 minutes. The mixture is transferred to a pre-chilled cuvette (0.2 cm gap, Bio-Rad). The DNA is transformed into bacteria by electroporation with Bio-Rad machine. (Setting: Volts: 2.25 KV; time: 5 ms; capacitance: 25 μF).

300 μl SOC medium is added to the cell mixture and bacteria are incubated at 30° C. shaker for one hour. A certain amount of culture is spread on LA plate with antibiotics and the plates were incubated at 30° C.

### EXAMPLE 18

### Transformation of Yeast Cells by Electroporation

One day before the experiment, 10 ml of YPD medium is inoculated with a single yeast colony of the strain to be transformed. It is grown overnight to saturation at 30° C. On the day of competent cell preparation, the total volume of yeast overnight culture is transferred to a 2 L baffled flask containing 500 ml YPD medium. The culture is grown with vigorous shaking at 30° C. to an OD600 reading of 0.8-1.0.

500 ml of culture is harvested by centrifuging at 4000×g, 4° C., for 5 min in autoclaved bottles. The supernatant is subsequently discarded. The cell pellet is washed in 250 ml cold sterile water. Washing is repeated twice. The supernatant is discarded.

The pellet is resuspended in 30 ml of ice-cold 1M Sorbitol. The suspension is transferred into a sterile 50 ml conical tube. The mixture is centrifuged in a GP-8 centrifuge 2000 rpm, 4° C. for 10 min. The supernatant is discarded. The pellet is resuspended in 50 μl of ice-cold 1M Sorbitol. The final volume of resuspended yeast should be 1.0 to 1.5 ml and the final OD600 should be ˜200.

In a sterile, ice-cold 1.5-ml microcentrifuge tube, 40 μl concentrated yeast cells are mixed with 1 μg of DNA contained in ˜5 μl. The mixture is transferred to an ice-cold 0.2-cm-gap disposable electroporation cuvette and pulsed at 1.5 kV, 25 μF, 200 □. It should be noted that the time constant reported by the Gene Pulser will vary from 4.2 to 4.9 msec. Times <4 msec or the presence of a current arc (evidenced by a spark and smoke) indicate that the conductance of the yeast/DNA mixture is too high.

400 μl ice-cold 1M sorbitol is added to the cuvette and the yeast is recovered, with gentle mixing. 200 μl aliquots of the east suspension should be spread directly on sorbitol selection plates. Incubate 3 to 6 days at 30° C. until colonies appear.

Literature Cited

1. Gibbs, J. B., Mechanism-Based Target Identification and Drug Discovery in Cancer Research. Science 2000, 287, 1969-73
2. Garret, M. D., Workman, P. Discovering Novel Chemotherapeutic Drugs for the Third Millennium. Eur. J. Cancer 1999, 35, 2010-30
3. Hanahan, et al., The Hallmarks of Cancer. Cell 2000, 100, 57-70
4. Druker, et al., Lessons learned from the development of an Abl tyrosine kinase inhibitor for chronic myelogenous leukemia. J. Clin. Invest. 2000, 105, 3-7
5. Sikic, B. I., New Approaches in cancer treatment. Ann. Onc. 1999, 10, S149-S153
6. Gibbs, J. B., Anticancer drug targets: growth factors and growth factor signaling. J. Clin. Invest. 2000, 105, 9-13
7. Drews, J., Drug Discovery: A historical perspective. Science 2000, 287, 1960-64
8. Harvey, A. L., Medicines from nature: are natural products still relevant to drug discovery? Trends Pharmacol. Sci. 1999, 20, 196-197
9. Cragg, G. M., Newman, D. J., Snader, K. M. Natural products in drug discovery and development. J. Nat. Prod. 1997, 60, 52-60
10. Verdine, G. L., The combinatorial chemistry of nature. Nature 1996, 384, 11-13
11. Demain, A. L., and J. E. Davies. Manual of industrial Microbiology and biotechnology; ASM Press: Washington, D.C., 1999
12. Mc Daniel, R., et al., Rational design of aromatic polyketide natural products by recombinant assembly of enzymatic subunits. Nature 1995, 375, 549-554
13. Jacobsen, J. R., D. E. Cane, and C. Khosla, Spontaneous priming of a downstream module in 6-deoxyerythronolide B synthase leads to polyketide biosynthesis. Biochem. 1998, 37, 4928-4934
14. Donadio, S., McAlpine, J. B., Sheldon, P. J., Jackson, M., and Katz, L., An erythromycin analog produced by reprogramming of polyketide synthesis. Proc. Natl. Acad. Sci. U.S.A. 1993, 90, 7119-23
15. Cortes, J. et al, Science, Repositioning of a domain in a modular polyketide synthase to promote specific chain cleavage 1995, 268, 1487-89
16. Amann, R. I. L. W., Schleifer K. H., Phylogenetic identification and in situ detection of individual microbial cells without cultivation. Microbiol. Rev. 1995, 59, 143-169
17. Robertson, D. E., et al. The discovery of new biocatalysts from microbial diversity. SIM News 1996, 46, 3-8
18. Stein, J. L., et al., Characterization of uncultivated prokaryotes: isolation and analysis of a 40-kilobase-pair genome fragment from a planktonic marine Archaeon. J. Bacteriol. 1996, 178, 591-599
19. Short, J. M., Recombinant approaches for accessing biodiversity. Nat. Biotechnol. 1997, 15, 1322-23
20. Sundberg, S. A., High-throughput and ultra-high-throughout screening: solution- and cell-based approaches. Curr. Opin. Biotech. 2000, 11, 47-53
21. Alvi, K. A., Pu, H., Asterriquinones produced by Aspergillus candidus inhibit binding of the Grb-2 adapter to phosphorylated EGF receptor tyrosine kinase. J. Antibiotics 1999, 52, 215-223
22. Levitzki, A., Gazit, A., Tyrosine Kinase inhibition: an approach to drug development. Science 1995, 267, 1782-88
23. Alberts, B., Bray, D., Lewis, J., Raff, M., Roberts, K., and J. D. Watson, Molecular biology of the cell; Garland Publishing, Inc.: New York, 1994
24. Kolibaba, K. S., Druker, B. J., Protein tyrosine kinases and cancer. Biochim Biophysica Acta 1997, 1333, F217-F248
25. Neal, D. E., Sharples, L., Smith, K., Fennelly, J., Hall, R. R., Harris, A. L., The epidermal growth factor receptor and the prognosis of bladder cancer. Cancer 1990, 65, 1619-25
26. Nicholson, S., Richard, J., Sainsbury, C., Halcrow, P., Kelly, P., Angus, B., Wright, C., Henry, J., Farndon, J., Harris, A., Epidermal growth factor receptor (EGFr) status associated with failure of primary endocrine therapy in elderly postmenopausal patients with breast cancer. Br. J. Cancer 1991, 63, 146-150
27. Klijn, J. G. M., Berns, P. M. J. J., Schmitz, P. I. M., Foekens, J. A., The clinical significance of epidermal growth factor receptor (EGF-R) in human breast cancer: a review on 5232 patients. Endocr. Rev. 1992, 12, 3-17
28. Hiesiger, E., Hayes, R., Pierz, D., Budzilovicb, G., Prognostic relevance of epidermal growth factor receptor (EGF-R) and c-neu/erbB2 expression in glioblastomas (GBMs). Neurooncol. 1993, 16, 93-104
29. Tateishi, M., Ishida, T., Mitsudomi, T., Kaneko, S., Sugimachi, K., Immunohistochemical evidence of autocrine growth factors in adenocarcinoma of the human lung Cancer Res. 1990, 50, 7077-80
30. Gorgoulis, V., Aninos, D., Mikou, P., Kanavaros, P., Karameris, A., Joardanoglu, J., Rasidakis, A., Veslemes, M., Ozanne, B., Spandidos, D. A., Expression of EGF, TGF-alpha and EGFR in squamous cell lung carcinomas Anticancer Res. 1992, 12, 1183-87
31. Sharif, T. R., Sharif, M., A high throughput system for the evaluation of protein kinase C inhibitors based on Elk1 transcriptional activation in human astrocytoma cells. Int. J. Onc. 1999, 14, 327-335
32. Li, Q., Vaingankar, S. M., Green, H. M., Green, M. M., Activation of the 9E3/cCAF chemokine by phorbol esters occurs via multiple signal transduction pathways that converge to MEK1/ERK2 and activate the Elk1 transcription factor. J Biol Chem 1999, 274, 15454
33. Treisman, R., Regulation of transcription by MAP kinase cascades. Curr. Opin. Cell Biol. 1996, 8, 205-215
34. Engler, D. A., Matsunami, R. K., Campion, S. R., Stringer, C. D., Stevens, A., Niyogi, S., Cloning of authentic human epidermal growth factor as a bacterial secretory protein and its initial structure-function analysis by site-directed mutagenesis. J. Biol. Chem. 1988, 263, 12384-390
35. Salmelin, C., Hovinen, J., Vilpo, J., Polymyxin permeabilization as a tool to investigate cytotoxicity of therapeutic aromatic alkylators in DNA repair-deficient Escherichia coli strains. Mut. Res. 2000, 467, 129-138
36. Gray, F., Kenney, J. S., Dunne, J. F., Secretion capture and report web: use of affinity derivatized agarose microdroplets for the selection of hybridoma cells. J. Immunol. Methods 1995, 182, 155-163
37. Powell, K. T., Weaver, J. C., Gel microdroplets and flow cytometry: rapid determination of antibody secretion by individual cells within a cell population. Bio/Technology 1990, 8, 333-337
38. Jan van der Wal, F., Luirink, J., Oudega, B., Bacteriocin release proteins: made of action, structure, and biotechnological application. FEMS Biol. Rev 1995, 17, 381-399
39. Majno, G., Joris, I., Apoptosis, oncosis, and necrosis: an overview of cell death. Am. J. Pathol. 1995, 146, 3-15
40. Wyllie, A. H., Kerr, J. F. R., Currie, A. R., Cell death; the significance of apoptosis. Int. Rev. Cytol. 1980, 68, 251-356
41. Sikic, B. I., Rozencweig, M., Carter, S. K., Eds. Bleomycin chemotherapy; Academic Press: Orlando, Fla., 1985
42. Deng, J L., Newman, D. J., Hecht, S. M., Use of COMPARE analysis to discover functional analogues of bleomycin. J. Nat. Prod. 2000, 63, 1269-72
43. Ortiz, L. A., Moroz, K., Liu, J Y., Hoyle, G. W., Hammond, T., Hamilton, R., Holian, A., Banks, W., Brody, A. R., Friedman, M., Alveolar macrophage apoptosis and TNF-a, but not p53, expression correlate with murine, response to bleomycin. Am. J. Physiol. 1998, 275, L1208-L1218
44. Kumagai, T., Sugiyama, M., Protection of mammalian cells from the toxicity of bleomycin by expression of a bleomycin-binding protein gene from streptomyces verticillus. J. Biochem. 1998, 124, 835-841
45. Benitez-Bribiesca, L., Sanchez-Suarez, P., Oxidative damage, bleomycin, and gamma radiation induce different types of DNA strand breaks in normal lymphocytes and thymocytes. Ann. NY Academy Sci. 1999, 887, 133-149
46. Du, L., Sanchez, C., Chen, M., Edwards, D. J., Shen, B., The biosynthetic gene cluster for the antitumor drug bleomycin from Streptomyces verticillus ATCC 15003 supporting functional interactions between nonribosomal peptide synthetases and a polyketide synthase. Chem. & Biol. 2000, 7, 623-642
49. Guiseley, K. B. U.S. Pat. No. 3,956,273, Modified Agarose and Agar and Methods of Making Same. May 11, 1976.
50. Phospholipids Handbook; Cevc, G., Ed.; Marcel Dekker: New York, 1993.
51. Ringsdorf, H.; Schlarb, B.; Venzmer, J. Molecular Architecture and Function of Polymeric Oriented Systems: Models for Study of Organization, Surface Recognition, and Dynamics of Biomembranes. Angew. Chem., Int. Ed. Engl. 1988, 27, 113-158 and references cited therein.
52. O'Brien, D. F.; Ramaswami, V. Polymerized Vesicles. Encycl. Polym. Sci. Eng. 1989, 17, 108-135.
53. Nilsson, K.; Brodelius, P.; Mosbach, K. Entrapment of Microbial and Plant Cells in Beaded Polymers. Methods in Emzymology, 1987, 135, 222-230 and references cited therein.
54. Kroger, N.; Deutzmann, R.; Sumper, M. Polycationic Peptides from Diatom Biosilica That Direct Silica Nanosphere Formation. Science 1999, 286, 1129-1132.
55. Cha, et al., Biomimetic Synthesis of Ordered Silica Structures Mediated by Block Copolypeptides. Nature 2000, 403, 289-292.
56. Bukanov, N. O., Demidov, V. V., Nielsen, P. E. & Frank-Kamenetskii, M. D. (1998). PD-loop: A complex of duplex DNA with an oligonucleotide. PNAS, 95 (10), 5516-5520.
57. Brenner, S., Williams, S. R., Vermaas, E. H., Storck, T., Moon, K., McCollum, C., Mao, J., Luo, S., Kirchner, J. J., Eletr, S., DuBridge, R. B., Burcham, T. & Albrecht, G. (1999). In vitro cloning of complex mixtures of DNA on microbeads: Physical separation of differentially expressed cDNAs. PNAS, 97 (4), 1665-1670.
58. Goryshin, I. Y., & Reznikoff, W. S. (1998). Tn5 in vitro transposition. J. Biol. Chem., 273, 7367-7374.
59. Jayasena, V. K. & Johnston, B. H. (1993). Complement-stabilized D-loop: RecA-catalyzed stable pairing of linear DNA molecules at internal sites. J. Mol. Biol., 230, 1015-1024.
60. Lohse, J., Dahl, O. & Nielsen, P. E. (1999). Double duplex invasion by peptide nucleic acid: A general principle for sequence-specific targeting of double-stranded DNA. PNAS, 96 (21), 11804-11808.
61. Sena, E. P. & Zarling, D. A. (1993). Targeting in linear DNA duplexes with two complementary probe strands for hybrid stability. Nature Genetics.

### EXAMPLE 19

### An Exemplary Novel High Throughput Cultivation Method

An aspect of the invention provides a novel high throughput cultivation method based on the combination of a single cell encapsulation procedure with flow cytometry that enables cells to grow with nutrients that are present at environmental concentrations. The resulting microcolonies can then be amplified by multiple displacement amplification for subsequent analysis.

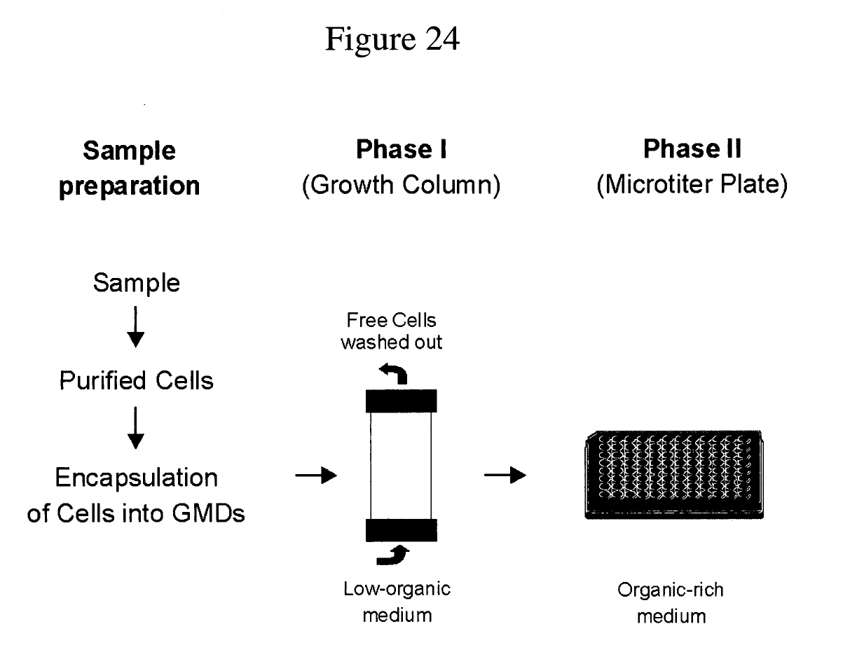

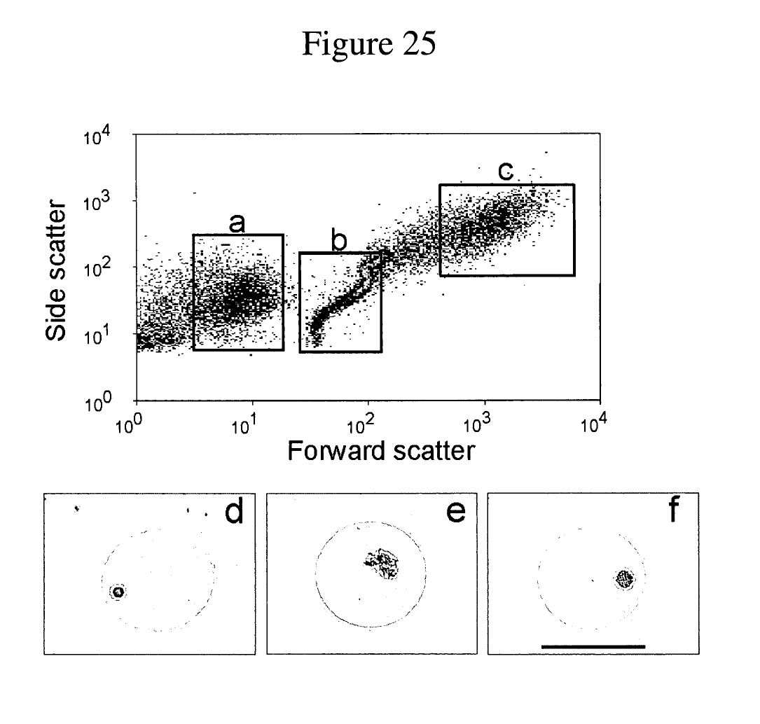

Seawater was collected from sites located in the Sargasso Sea. Individual cells were concentrated from this seawater by tangential flow filtration and encapsulated in gel microdroplets (GMD). Similar GMDs have been used previously to grow bacteria¹² and for screening purposes¹³⁻¹⁵. Single encapsulated cells (see Methods) were transferred into chromatography columns (referred to henceforth as growth columns). Different culture media selective for aerobic, nonphototrophic organisms were pumped through the growth columns containing 10 million GMDs (FIG. 24). The pore size of the GMDs allows the free exchange of nutrients. The encapsulated microorganisms were able to divide and form microcolonies of approximately 20 to 100 cells within the GMDs. Based on their distinctive light scattering signature, these microcolonies were detected and separated by flow cytometry at a rate of 5,000 GMDs per second. The increase in forward and side scatter was shown by microscopy to be directly proportional to the size of the microcolony grown within the GMD. This property enabled discrimination between unencapsulated single cells, empty or singly occupied GMDs, and GMDs containing a microcolony (FIG. 25).

To determine the optimal growth medium for a broad diversity of organisms, four media were tested in the growth columns: Organic rich medium diluted in seawater (marine medium); seawater amended with a mixture of amino acids; seawater amended with inorganic nutrients; and sterile filtered seawater (FIG. 24). After five weeks of incubation, 1200 GMDs, each containing a microcolony, were collected by flow cytometry from each of the four growth columns. A 16S rRNA gene clone library was generated from each group of 1200 microcolonies and analysed. In diluted marine medium, only four bacterial species were identified, belonging to the genera Vibrio, Marinobacter or Cytophaga, all common sea water bacteria that have been cultivated previously^{3,9}. The media containing amino acids or inorganic minerals revealed slightly more diversity. Analysis of 50 clones derived from each medium yielded twelve different bacterial species from the amino acid supplemented medium, and eleven species from the inorganic medium. Filtered seawater alone (taken from the original sampling site) yielded the highest biodiversity (39 species out of 50 clones analysed), with many different phylogenetic groups represented. These results demonstrated that organisms capable of rapid growth outgrew their more fastidious neighbours in the presence of organic rich medium.

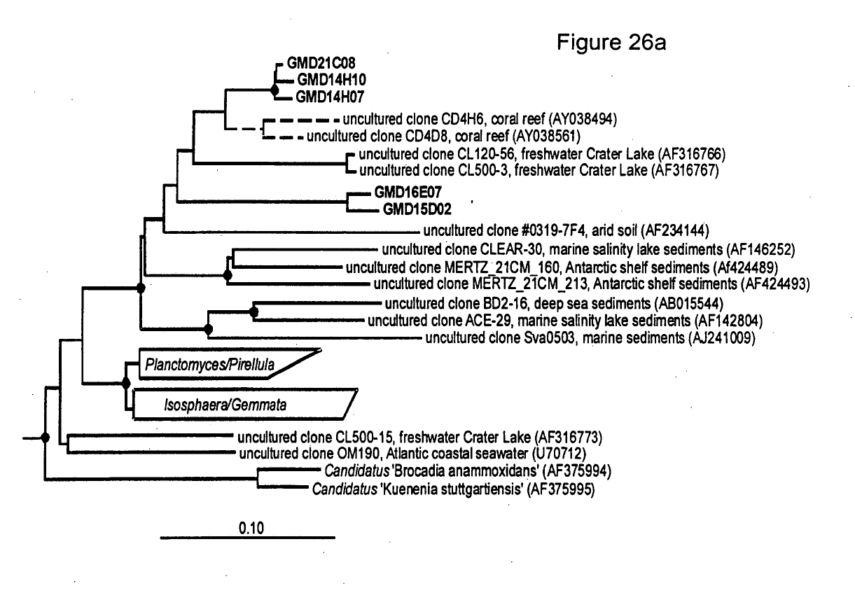

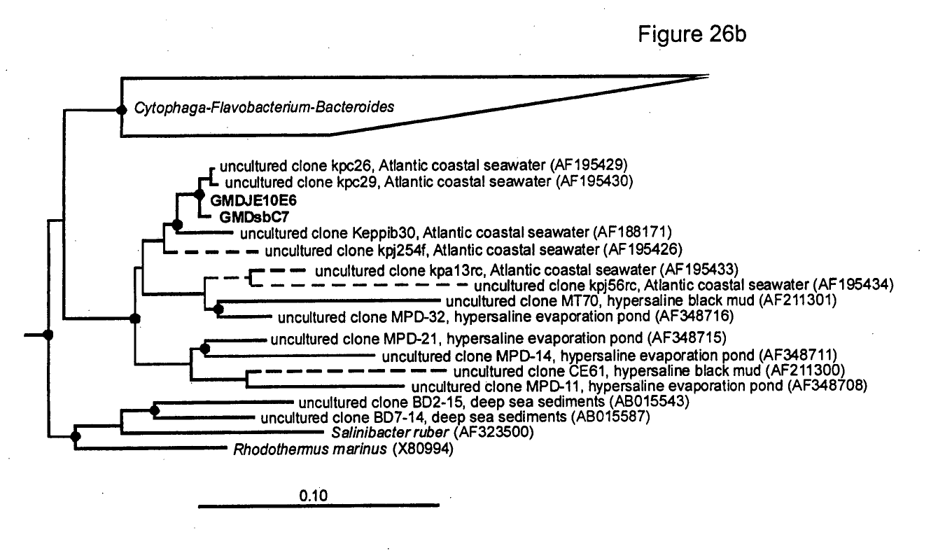

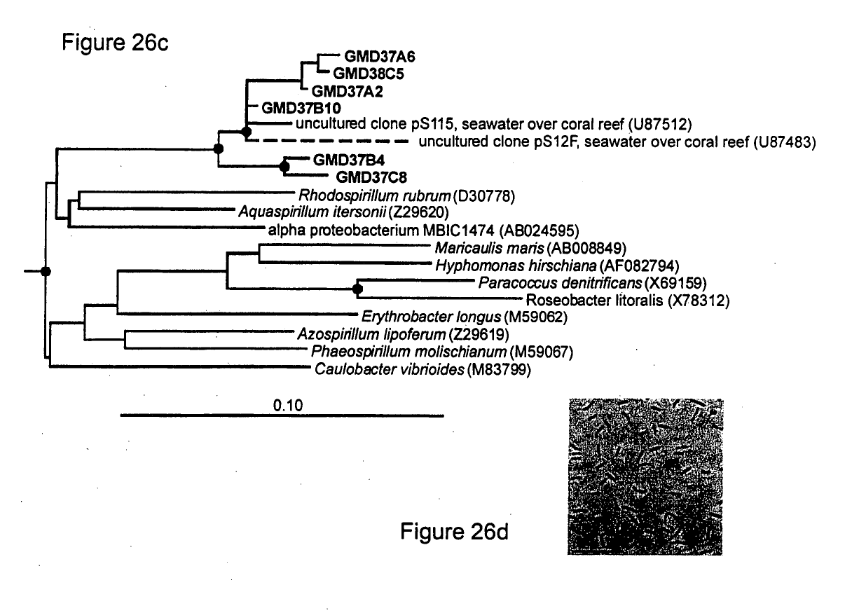

Growth columns were next inoculated with GMDs again generated from samples obtained from the Sargasso Sea, but now using only filtered seawater as growth medium. From each of two growth columns, 500 GMDs containing microcolonies were sorted, and the 16S rRNA genes contained therein were amplified by PCR. A 16S rRNA gene library was also constructed from the original environmental sample from which the microorganisms were obtained for encapsulation. Most of the environmental 16S rRNA sequences derived from this latter sample fell within the nine common bacterioplankton groups^{3,11}. In contrast, many of the 150 16S rRNA gene sequences obtained from the microcolonies fell into clades which contain no previously cultivated representatives (see supplementary information). Three of the most notable examples, described in more detail below, were clades affiliated with the Planctomycetes and relatives, the Cytophaga-Flavobacterium-Bacteroides and relatives, and the alpha subclass of Proteobacteria (FIG. 26). None of these groups were detected within the environmental 16S rRNA gene clone library (167 clones analysed).

Five microcolony 16S rRNA gene sequences were related to the Planctomycetales, one of the main phylogenetic branches of the domain Bacteria³ (FIG. 26a). Sequencing of cloned rRNA genes from marine environments had previously revealed several new, apparently uncultivated phylotypes within the Planctomycetales¹⁶⁻¹⁸. Many of these new phylotypes fall within a single, highly diverse monophyletic clade that, prior to this study, contained no cultivated representatives. The five Planctomycetales-related microcolonies identified in this study form two separate lineages within this deep branching Planctomycetales clade (FIG. 26a). One lineage, represented by sequences GMD21C08, GMD14H10, and GMD14H07 (FIG. 26a), was most closely related to 16S rRNA gene clone sequences recovered from bacteria associated with marine corals (84.9-89.2% similar)¹⁷. The second lineage, represented by GMD16E07 and GMD15D02 (FIG. 26a), form a unique line of desent within this clade, and are <84% similar to all previously published 16S rRNA gene sequences.

Two microcolony 16S rRNA gene sequences fell within the Cytophaga-Flavobacterium-Bacteroides and their relatives. These two closely related sequences form a lineage within a cluster of gene clone sequences from predominantly marine and hypersaline environments¹⁹⁻²¹. This cluster occupies one of the deepest phylogenetic branches of the Cytophaga-Flavobacterium-Bacteroides and relatives group; only the Rhodothermus/Salinibacter lineage is deeper²⁰. Within this cluster, the two microcolony gene sequences were nearly identical (>99% similar) to environmental 16S rRNA gene clone sequences obtained from seawater collected off of the Atlantic coast of the United States²¹ (FIG. 26b). Analysis of Phase II cultures (see later) obtained from these sorted microcolonies (FIG. 24) revealed a culture (strain GMDJE10E6) with an identical 16S rRNA gene sequence that reached an optical density (OD₆₀₀ₙₘ) of 0.3 (FIG. 26d).

A cluster of six microcolonies was recovered that was phylogenetically affiliated with a previously uncultivated lineage of 16S rRNA gene clone sequences within the alpha subclass of the Proteobacteria (FIG. 26c). The microcolony sequences formed two subclusters; one was closely related to two 16S rRNA gene clone sequences recovered from marine samples taken from a coral reef (95.1-98.6% similar) (GenBank U87483 and U87512); the second was moderately related to the same coral reef-associated environmental gene clones (87.9-95.7% similar).

Thus, the application of this novel high throughput cultivation method resulted in the growth and isolation of several bacteria representing previously uncultured phylotypes (see supplementary information). This reflects the ability of GMDs to permit the simultaneous and non-competitive growth of both slow and fast growing microorganisms in media with very low substrate concentrations. The physical separation of cells (contained in the GMDs within the growth columns), combined with flow cytometry isolation of microcolonies at different times of incubation, enabled the cultivation of a broad range of bacteria, and prevented over-growth by the fast growing microorganisms (the “microbial weeds”)⁹.

To test if this novel high throughput cultivation method is applicable to different environments, we applied the technology to an alkaline lake sediment (Lake Bogoria, Kenya, data not shown) and to a soil sample (Ghana). Microorganisms from the soil sample were separated from the soil matrix, encapsulated and incubated in the growth column under aerobic conditions in the dark. Diluted soil extract, obtained from the same sample, was used as growth medium. The microcolonies were analysed by 16S rRNA gene sequencing. To cater for bacteria with disparate growth rates, microcolonies were separated from the growth column by flow cytometry at different time points. 16S rRNA gene sequence analysis revealed that many phylogenetically different microorganisms could be cultivated within the GMDs in Phase I (FIG. 24) (see supplementary information). This approach can be extended to many other physiological and environmental conditions. For example, it was demonstrated that encapsulated cells of Methanococcus thermolithotrophicus can grow and form microcolonies within GMDs when incubated under strictly anaerobic conditions.

Physiological studies, natural product screening or studies of cell-cell interaction require the ability to grow microorganisms to a certain cell mass. Therefore we designed experiments to determine if these microcolonies are able to serve as inocula for larger scale microbial cultures (FIG. 24, Phase II). Encouragingly, earlier microscopic analysis had revealed that encapsulated bacteria could indeed grow out of GMDs when provided with a rich supply of nutrients. GMDs were obtained from a soil sample (Ghana), as described above. After growth in diluted soil extract medium, microcolonies were sorted into organic rich medium (FIG. 24, Phase II). A total of 960 GMDs containing microcolonies, each derived from a single organism, were sorted into 96 well microtitre plates filled with organic rich medium (1 GMD per well). The 960 cultures were analysed for growth by measuring optical densities (OD₆₀₀ₙₘ). After one week of incubation, 67% of the cultures showed turbidity above OD 0.1, corresponding to at least 10⁷ cells per millilitre. Cell densities were high enough to permit the detection of antifungal activity among some of the cultures (data not shown). To analyse the diversity within these cultures in more detail, 100 randomly picked cultures were analysed by 16S rRNA gene sequencing, revealing many different species (see supplementary information). The remaining 33% of the cultures that did not grow to measurable densities (fewer then 10⁶ cells per millilitre), showed bacterial growth when assessed microscopically. This is consistent with recent reports indicating that certain bacteria do not grow to cell densities greater than 10⁶ cells per millilitre¹¹.

In order to maintain and access microcolonies for physiological studies, we evaluated the minimal number of cells required for passaging by re-encapsulation and detection by flow cytometry. Flow cytometry analysis of 1000 and 100 individually encapsulated cells resulted in the detection of 360 and 15 microcolonies, respectively. Even when using cultures comprising just 10 bacterial cells, this method allowed recovery of, on average, one viable bacterial culture. This experiment demonstrates that it is possible to transfer, and therefore maintain, a culture of 100 cells derived directly from a microcolony.

GMDs separate microorganisms from each other, while still allowing the free flow of signalling molecules between different microcolonies. Therefore, this method might be applicable for the analysis of interactions between different organisms under in situ conditions, for example by inserting the encapsulated cells back into the environment (e.g. the open ocean). The simultaneous encapsulation of more than one cell (prokaryotic as well as eukaryotic) into one GMD might also be used to mimic conditions found in nature, allowing analysis of cell-cell interactions. Another advantage of this technology is the very sensitive detection of growth. This high throughput cultivation method allows the detection of microcolonies containing as few as 20 to 100 cells. Nutrient sparse media, such as seawater, were sufficient to support growth, and yet their carbon content was low enough to prevent “microbial weeds” from overgrowing slow growing microorganisms. We have demonstrated that this technology can be used to culture thus far uncultivated microorganisms. The microcolonies obtained can then be used as inocula for further cultivation.

In combination with rRNA analysis and mixed organism recombinant screening approaches^{22,23}, this technology will permit a more complete understanding of unexplored microbial communities. It will find applications in environmental microbiology, whole cell optimisation, and drug discovery. The combination of cultivation with direct DNA amplification from microcolonies will undoubtedly contribute to a broader understanding of microbial ecology by linking microbial diversity with metabolic potential.

Methods

Sample Collection

Water samples were collected in the Sargasso Sea (31°50′ N 64°10′W and 32°05′ N 64°30′W) at depths of 3 m and 300 m. For each sample, a volume of 130 l was concentrated by tangential flow filtration. Soil samples were collected from tropical forest (05°56′N 00°03′) and chaparral (05°55′N 00°03′W) in Ghana and combined in equal amounts. Cells were separated from the soil matrix by repeated sheering cycles followed by density gradient centrifugation²⁴.

Cell Encapsulation and Growth Conditions

Concentrated cell suspensions were used for encapsulation. Single occupied gel microdroplets (GMDs) were generated by using a CellSys 100™ microdrop maker (OneCell System) according to the manufacturer's instructions. Encapsulation of single cells was monitored by microscopy. The GMDs were dispensed into sterile chromatography columns XK-16 (Pharmacia Biotec) containing 25 ml of media. Columns were equipped with two sets of filter membranes (0.1 μm at the inlet of the column and 8 μm at the outlet). The filters prevented free-living cells contaminating the media reservoir and retained GMDs in the column while allowing free-living cells to be washed out.

Media were pumped through the column at a flow rate of 13 ml/h. Media used for incubation of marine samples were: Sargasso Sea water filter sterilized (SSW); SSW amended with NaNO₃ (4.25 g/l), K₂HPO₄ (0.016 g/l), NH₄Cl (0.27 g/l), trace metals and vitamins²⁵; SSW amended with amino acids at concentrations between 6 to 30 nM²⁶ and marine medium (R2A, Difco) diluted in SSW (1:100, vol/vol). Soil extracts were prepared as previously described²⁷ and added to the media at final concentrations of 25 to 40 ml/l in 0.85% NaCl (vol/vol). GMDs were incubated in the columns for a period of at least 5 weeks. Microcolonies that were sorted individually into 96 well microtitre plates were grown with marine medium (R2A, Difco) in SSW or with soil extracts amended with glucose, peptone, and yeast extract (1 g/l) and humic acids extract 0.001% (vol/vol).

Flow Cytometry

GMDs containing colonies were separated from free-living cells and empty GMDs by using a flow cytometer (MoFlo, Cytomation). Precise sorting was confirmed by microscopy. For the re-encapsulation experiment, a series of 1000, 100 and 10 Escherichia coli cells (expressing a green fluorescent protein, ZsGreen, Clontech), were individually encapsulated and incubated for three hours to form microcolonies within the GMDs. GMDs were analysed by flow cytometry and sorted.

Phylogenetic Analysis

Ribosomal RNA genes from environmental samples, microcolonies and cultures were amplified by PCR using general oligonucleotide primers (27F and 1392R) for the domain Bacteria. To avoid nonspecific amplification, PCR reactions were irradiated with an UV Stratalinker (Stratagene) at maximum intensity prior to template addition. After cloning (TOPO-TA, Invitrogen), inserts were screened by their restriction pattern obtained with AvaI, BamHI, EcoRI, HindIII, KpnI, and XbaI. Nearly full length 16S rRNA gene sequences were obtained and added to an aligned database of over 12,000 homologous 16S rRNA primary structures maintained with the ARB software package²⁸. Phylogenetic relationships were evaluated using evolutionary distance, parsimony, and maximum likelihood methods, and were tested with a wide range of bacterial phyla as outgroups²⁹. Hypervariable regions were masked from the alignment. The phylogenetic trees shown in FIG. 26 demonstrates the most robust relationships observed, and was determined using evolutionary distances calculated with the Kimura 2-parameter model for nucleotide change and neighbour-joining. Bootstrap proportions from 1000 resamplings were determined using both evolutionary distance and parsimony methods. Short reference sequences were added to the phylogenetic trees with the parsimony insertion tool of ARB, and are indicated by dotted lines.

References

1. Pace, N. R. A molecular view of microbial diversity and the biosphere. Science 276, 734-740 (1997).
2. Amann, R. I., Ludwig, W. & Schleifer, K.-H. Phylogenetic identification and in situ detection of individual microbial cells without cultivation. Microbiol Rev 59, 143-169 (1995).
3. Giovannoni, S. J. & Rappé, M. in Microbial Ecology of the Ocean (ed. Kirchman, D. L.) 47-84 (Wiley-Liss Inc., 2000).
4. Fuhrman, J. A., McCallum, K. & Davis, A. A. Phylogenetic diversity of subsurface marine microbial communities from the Atlantic and Pacific Oceans. Appl Environ Microbiol 59, 1294-1302 (1993).
5. Kaeberlein, T., Lewis, K. & Epstein, S. S. Isolating “uncultivable” microorganisms in pure culture in a simulated natural environment. Science 296, 1127-1129 (2002).
6. Beja, O. et al. Bacterial rhodopsin: evidence for a new type of phototrophy in the sea. Science 289, 1902-1906 (2000).
7. Beja, O. et al. Unsuspected diversity among marine aerobic anoxygenic phototrophs. Nature 415, 630-633 (2002).
8. Ferguson, R. L., Buckley, E. N. & Palumbo, A. V. Response of marine bacterioplankton to differential filtration and confinement. Appl Environ Microbiol 47, 49-55 (1984).
9. Eilers, H., Pemthaler, J., Glöckner, F. O. & Amann, R. Culturability and in situ abundance of pelagic bacteria from the North Sea. Appl Environ Microbiol 66, 3044-3051 (2000).
10. Xu, H. S. et al. Survival and viability of nonculturable Escherichia coli and Vibrio cholerae in the estuarine and marine environment. Microb Ecol 8, 313-323 (1982).
11. Rappé, M. S., Connon, S. A., Vergin, K. L. & Giovannoni, S. J. Cultivation of the ubiquitous SAR11 marine bacterioplankton clade. Nature In press (2002).
12. Manome, A. et al. Application of gel microdroplet and flow cytometry techniques to selective enrichment of non-growing bacterial cells. FEMS Microbiol Lett 197, 29-33 (2001).
13. Short, J. M. & Keller, M. High throughput screening for novel enzymes. U.S. Pat. No. 6,174,673B1 (2001).
14. Powell, K. T. & Weaver, J. C. Gel microdroplets and flow cytometry: rapid determination of antibody secretion by individual cells within a cell population. Bio/Technology 8, 333-337 (1990).
15. Ryan, C., Nguyen, B. T. & Sullivan, S. J. Rapid assay for mycobacterial growth and antibiotic susceptibility using gel microdrop encapsulation. J Clin Microbiol 33, 1720-1726 (1995).
16. Bowman, J. P., Rea, S. M., McCammon, S. A. & McMeekin, T. A. Diversity and community structure within anoxic sediment from marine salinity meromicitc lakes and a coastal meromictic marine basin, Vestfold Hilds, Eastern Australia. Environ Microbiol 2, 227-237 (2000).
17. Frias-Lopez, J., Zerkle, A. L., Bonheyo, G. T. & Fouke, B. W. Partitioning of bacterial communities between seawater and healthy, black band diseased, and dead coral surfaces. Appl Environ Microbiol 68, 2214-2228 (2002).
18. Ravenschlag, K., Sahm, K., Pernthaler, J. & Amann, R. High bacterial diversity in permanently cold marine sediments. Appl Environ Microbiol 65, 3982-3989 (1999).
19. Tanner, M. A., Everett, C. L., Coleman, W. J., Yang, M. M. & Youvan, D. C. Complex microbial communities inhabiting sulfide-rich black mud from marine coastal environments. Biotechnology et alia 8, 1-16 (2000).
20. de Souza, M. P. et al. Identification and characterization of bacteria in a selenium-contaminated hypersaline evaporation pond. Appl Environ Microbiol 67, 3785-3794 (2001).
21. Kelly, K. M. & Chistoserdov, A. Y. Phylogenetic analysis of the succession of bacterial communities in the Great South Bay (Long Island). FEMS Microbiol Ecol 35, 85-95 (2001).
22. Short, J. M. Recombinant approaches for accessing biodiversity. Nature Biotechnology 15, 1322-1323 (1997).
23. Robertson, D. E., Mathur, E. J., Swanson, R. V., Marrs, B. L. & Short, J. M. The discovery of new biocatalysts from microbial diversity. SIM News 46, 3-8 (1996).
24. Fægri, A., Torsvik, V. L. & Goksöyr, J. Bacterial and fungal activities in soil: separation of bacteria and fungi by a rapid fractionated centrifugation technique. Soil Biol Biochem 9, 105-112 (1977).
25. Widdel, F. & Bak, F. in The Prokaryotes (eds. Balows, A., Trüper, H. G., Dworkin, M., Harder, W. & Schleifer, K.-H.) 3352-3392 (Springer-Verlag, New York, 1992).
26. Ouverney, C. C. & Fuhrman, J. A. Marine planktonic archaea take up amino acids. Appl Environ Microbiol 66, 4829-4833 (2000).
27. Vobis, G. in The Prokaryotes (eds. Balows, A., Trüper, H. G., Dworkin, M., Harder, W. & Schleifer, K.-H.) 1029-1060 (Springer-Verlag, New York, 1992).
28. Strunk, O. & Ludwig, W. in http://www.mikro.biologie.tu-muenchen.de (Department of Microbiology, Technische Universität München, Munich, Germany, 1998).
29. Ludwig, W. et al. Detection and in situ identification of representatives of a widely distributed new bacterial phylum. FEMS Microbiol Lett 153, 181-190 (1997).

### EXAMPLE 20

### Amplification of Trace Amounts of Environmental gDNA

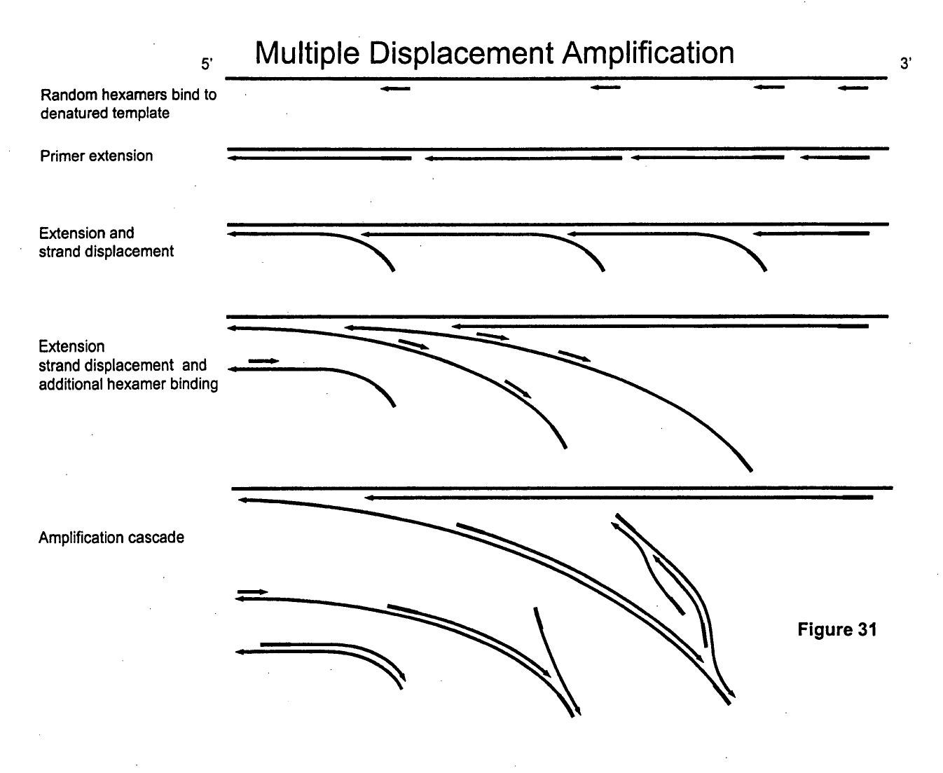

FIG. 31 shows a schematic diagram of the procedure used to amplify trace amounts of environmental gDNA. The amplification proceeded as follows.

Template Preparation. Trace amounts of environmental, large fragment gDNA were encased in agarose. The agarose gel piece was then equilibrated by adding agarase buffer and incubating at room temperature for 1 hour. After removing the buffer, the agarose was melted by incubating at 70° C. for 15 minutes. The melted agarose was then digested with agarase by incubating at 40° C. overnight. Approximately 1 μl (or 1-100 ng) of this solution was used as the template for the amplification reaction. The solution can also be concentrated by ethanol or isopropanol precipitation, then used as the template for the amplification reaction.

Amplification. 1-100 ng of the template was added to random primers (random 7-mers with an additional two nitroindole residues at the 5′ end and a phosphorothioate linkage at the 3′ end; GC-rich random hexamers can be added when template is GC-rich) at 100 μM final concentration in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration). The template was denatured by incubating the solution at 95° C. for 3 minutes followed by cooling on ice. After cooling, deoxynucleoside triphosphates (dNTP) (100 μM final concentration), and Phi29 polymerase (Molecular Staging (1 μL in a 50 μL reaction), Amersham (1 μL in a 20 μL reaction)) in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration) was added. The entire solution was incubated at 30° C. for 3-16 hours. Partway through the incubation period, extra dNTP, primers, and/or buffer may be added to increase the size of the product. Following amplification, the enzyme was heat inactivated at 65° C. for 10 minutes.

Numerous modifications and variations of the present invention are possible in light of the above teachings; therefore, within the scope of the claims, the invention may be practiced other than as particularly described.

### EXAMPLE 21

### Amplification of Trace Amounts of Environmental gDNA

A) Cut and Ligate Method:

Template Preparation. Trace amounts of whole E. coli cells, were encased in an agarose noodle, treated with lysozyme, proteinaseK, melted and digested with agarase. Preparation of the restriction digest may be done by any means known to those skilled in the art. The method used here to prepare the restriction digest was to mix 5 uL of the template DNA, 1 uL EcoRI Buffer (commercially available from New England BioLabs), 0.5 uL EcoRI (commercially available from New England BioLabs), and 3.5 uL H₂0. The sample was incubated at 37° C. for between 1-16 hours. The restriction enzyme was heat-inactivated at 65° C. for 20 minutes. 1 uL T4 DNA Ligase (commercially available from New England BioLabs) and 0.56 uL 20 mM ATP (commercially available from Sigma) was added directly to the reaction. The sample was incubated at room temperature for between 1-16 hours. The template DNA is very dilute so that the DNA fragments will preferentially form self-ligated products (circles). The ligase was heat-inactivated at 65° C. for 10 minutes. Approximately 2 uL was used directly as template for amplification. FIG. 32 shows the number of cells detectable as template resulting from this experiment.

Amplification. Approximately 2 uL of the template was added to random primers (random 7-mers with an additional two nitroindole residues at the 5′ end and a phosphorothioate linkage at the 3′ end; GC-rich random hexamers can be added when template is GC-rich) at 100 M final concentration in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration). The template was denatured by incubating the solution at 95° C. for 3 minutes followed by cooling on ice. After cooling, deoxynucleoside triphosphates (dNTP) (100 μM final concentration), and Phi29 polymerase (Molecular Staging (1 μL in a 50 μL reaction), Amersham (1 μL in a 20 μL reaction)) in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration) was added. The entire solution was incubated at 30° C. for 3-16 hours. Partway through the incubation period, extra dNTP, primers, and/or buffer may be added to increase the yield of the product. Following amplification, the enzyme was heat inactivated at 65° C. for 10 minutes.

Samples were evalutated using GeneChip® E. coli Antisense Genome Array technology (commercially available from Affymetrix).

Numerous modifications and variations of the present invention are possible in light of the above teachings; therefore, within the scope of the claims, the invention may be practiced other than as particularly described.

References:

1) Lage, et al., Whole Genome Analysis of Genetic Alterations in Small DNA Samples Using Hyperbranched Strand Displacement Amplification and Array-CGH, Genome Research, 13:294-307 (2003).

2) Detter, et al., Isothermal Strand-Displacement Amplification Applications for High-Throughput Genomics, Genomics, Vol. 80, No.6 (Decmeber 2002).

### EXAMPLE 22

### Amplification of Trace Amounts of Environmental gDNA

B) Shear and Ligate Method:

Template Preparation. Trace amounts of environmental whole cells, are encased in an agarose noodle, treated with lysozyme, proteinaseK, melted and digested with agarase. The template DNA will be sheared by a shearing means (e.g., shearing machine (GeneMachines Hydroshear), 25 gauge needle, among others) known by those skilled in the art. The DNA ends will be filled in with a DNA polymerase. The DNA will be blunt ligated with T4 DNA Ligase. The ligated DNA will be used as the template for amplification.

Amplification. 1-50 uL of the template is added to random primers (random 7-mers with an additional two nitroindole residues at the 5′ end and a phosphorothioate linkage at the 3′ end; GC-rich random hexamers can be added when template is GC-rich) at 100 μM final concentration in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration). The template is denatured by incubating the solution at 95° C. for 3 minutes followed by cooling on ice. After cooling, deoxynucleoside triphosphates (dNTP) (100 μM final concentration), and Phi29 polymerase (Molecular Staging (1 μL in a 50 μL reaction), Amersham (1 μL in a 20 μL reaction)) in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration) will be added. The entire solution will be incubated at 30° C. for 3-16 hours. Partway through the incubation period, extra dNTP, primers, and/or buffer may be added to increase the yield of the product. Following amplification, the enzyme will be heat inactivated at 65° C. for 10 minutes.

Samples will be evalutated using GeneChip® E. coli Antisense Genome Array technology (commercially available from Affymetrix).

Numerous modifications and variations of the present invention are possible in light of the above teachings; therefore, within the scope of the claims, the invention may be practiced other than as particularly described.

### EXAMPLE 23

### Amplification of Trace Amounts of Environmental gDNA

Re-amplification Method:

In another aspect, the amplification process presented above may be performed iteratively on the whole amplification product from the previous amplification step. The template DNA may be prepared by any technique known by those skilled in the art.

Amplification. 50 picograms-5 ng of the E. coli DNA template was added to random primers (random 7-mers with an additional two nitroindole residues at the 5′ end and a phosphorothioate linkage at the 3′ end; GC-rich random hexamers can be added when template is GC-rich) at 100 μM final concentration in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration). The template was denatured by incubating the solution at 95° C. for 3 minutes followed by cooling on ice. After cooling, deoxynucleoside triphosphates (dNTP) (100 μM final concentration), and Phi29 polymerase (Molecular Staging (1 μL in a 50 μL reaction), Amersham (1 μL in a 20 μL reaction)) in 1× Buffer Y+/Tango™ (3.3 mM Tris-acetate (pH 7.9 at 37° C.), 1 mM magnesium acetate, 6.6 mM potassium acetate, 10 μg/ml BSA) (MBI Fermentas) plus Tween (0.12% final concentration) is added. The entire solution is incubated at 30° C.

After 3 hours, the reaction components (minus additional template) were added again to the solution and incubated for an additional 3 hours. After the additional at least 1 hour, the reaction components (minus additional template) were added again to the solution and incubated an additional 3 hour3. The additional components, and additional incubations allowed otherwise unamplifiable samples to be amplified.

Samples will be evalutated using GeneChip® E. coli Antisense Genome Array technology (commercially available from Affymetrix).

Numerous modifications and variations of the present invention are possible in light of the above teachings; therefore, within the scope of the claims, the invention may be practiced other than as particularly described.

TABLE 1

A2
Fluorescein conjugated casein (3.2 mol fluorescein/mol casein)
CBZ—Ala—AMC
t-BOC—Ala—Ala—Asp—AMC
succinyl-Ala—Gly—Leu—AMC
CBZ—Arg—AMC
CBZ—Met—AMC
morphourea-Phe—AMC
t-BOC = t-butoxy carbonyl, CBZ = carbonyl benzyloxy.
AMC = 7-amino-4-methyl coumarin
AD3
Fluorescein conjugated casein
t-BOC—Ala—Ala—Asp—AFC
CBZ—Ala—Ala—Lys—AFC
succinyl-Ala—Ala—Phe—AFC
succinyl-Ala—Gly—Leu—AFC
AFC = 7-amino-4-trifluoromethyl coumarin)
AE3
Fluorescein conjugated
casein
AF3
t-BOC—Ala—Ala—Asp—AFC
CBZ—Asp—AFC
AG3
CBZ—Ala—Ala—Lys—AFC
CBZ—Arg—AFC
AH3
succinyl-Ala—Ala—Phe—AFC
CBZ—Phe—AFC
CBZ—Trp—AFC
AI3
succinyl-Ala—Gly—Leu—AFC
CBZ—Ala—AFC
CBZ—Sewr—AFC

TABLE 2
L2

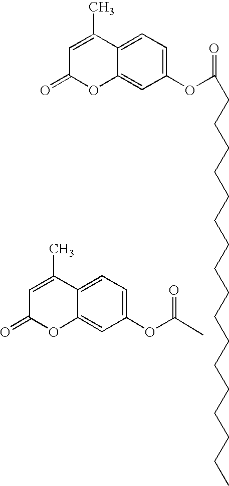

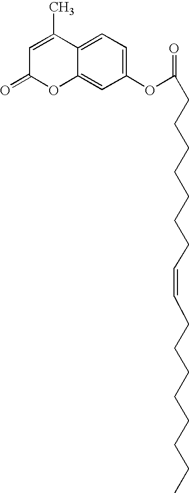

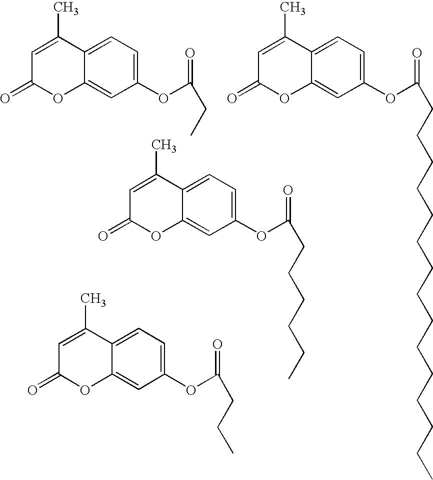

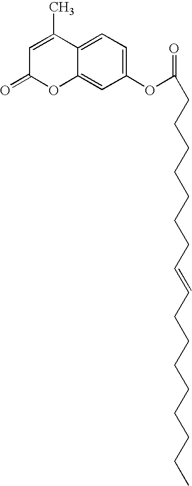

LA3

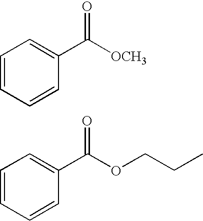

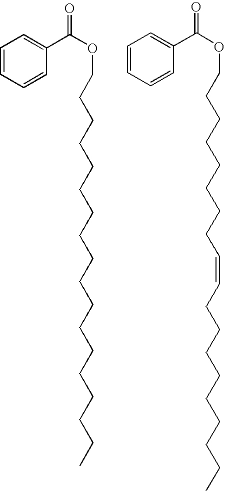

LB3

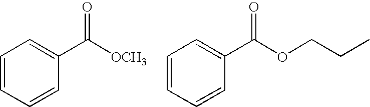

LD3

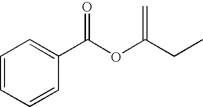

LF3

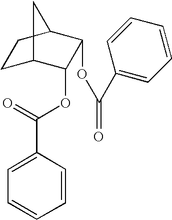

LC3

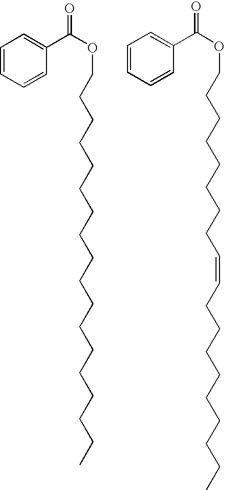

LE3

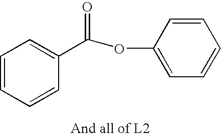

LG3

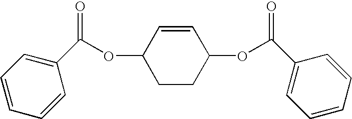

TABLE 3
LH3

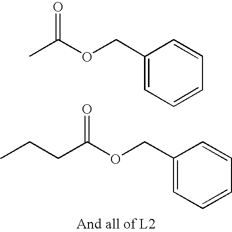

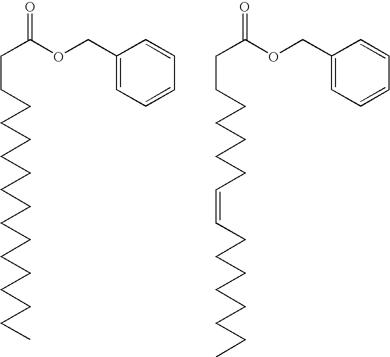

LI3

| | |
|---|---|
| 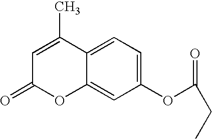 | 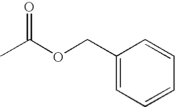 |
| 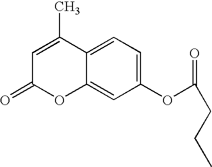 | 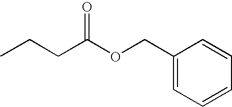 |
| 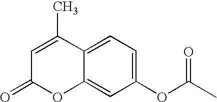 | 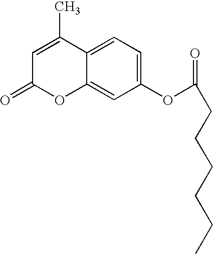 |

LJ3

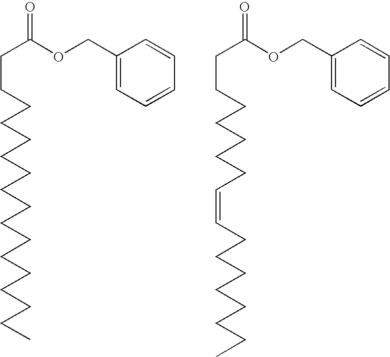

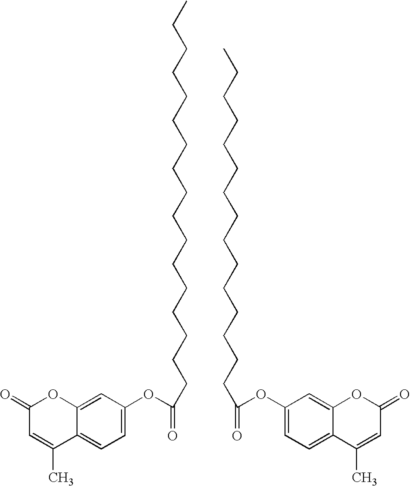

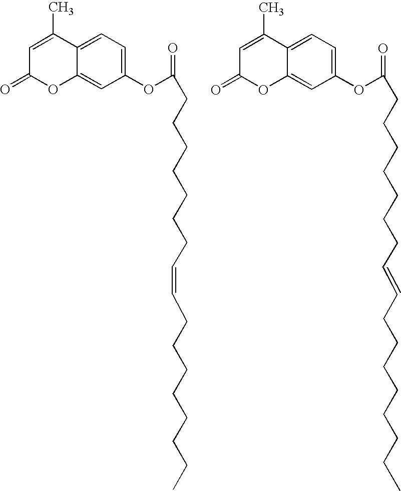

LK3

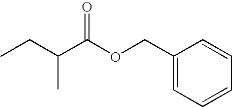

LM3

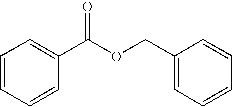

LL3

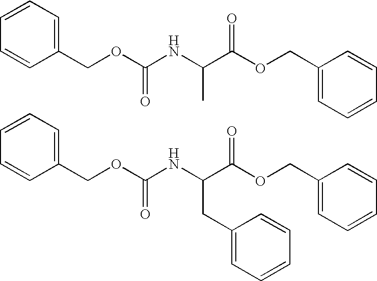

LN3

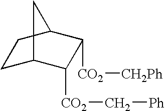

LO3

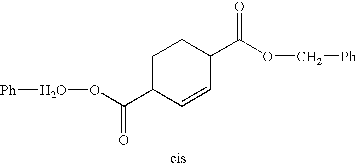

TABLE 4
4-methyl umbelliferone
wherein R =

| | |
|---|---|
| G2 | β-D-galactose |
| | β-D-glucose |
| | β-D-glucuronide |
| GB3 | β-D-cellotrioside |
| | β-D-cellobiopyranoside |
| GC3 | β-D-galactose |
| | α-D-galactose |
| CD3 | β-D-glucose |
| | α-D-glucose |
| GE3 | β-D-glucuronide |
| GI3 | β-D-N,N-diacetylchitobiose |
| GJ3 | β-D-fucose |
| | α-L-fucose |
| | β-L-fucose |
| GK3 | β-D-mannose |
| | α-D-mannose |

non-Umbelliferyl substrates

| | |
|---|---|
| GA3 | amylose [polyglucan α 1,4 linkages], amylopectin |
| | [polyglucan branching α 1,6 linkages] |
| GF3 | xylan [poly 1,4-D-xylan] |
| GG3 | amylopectin, pullulan |
| GH3 | sucrose, fructofuranoside |

What is claimed is:

1. A method for amplifying a DNA template from trace amounts of DNA derived from at least one species of organism comprising:
a) obtaining trace amounts of cDNA, gDNA, or genomic DNA fragments from at least one species of organism;
b) preparing a template from said cDNA, gDNA, or genomic DNA fragments; and
c) amplifying the template.

2. The method of claim 1, wherein said template is fragmented.

3. The method of claim 1, wherein said trace amounts of cDNA, gDNA, or genomic DNA fragments are partially or completely digested.

4. The method of claim 2, wherein the template fragmentation is achieved by enzymatic, chemical, photometric, mechanical or any means that provides segments.

5. The method of claim 4, wherein the enzymatic fragmentation comprises use of a DNase or a restriction enzyme.

6. The method of claim 4, further comprising filling DNA ends by polymerase extension.

7. The method of claim 1, wherein said template is diluted to a degree sufficient to obtain substantially self-ligated products in the presence of ligase and ligase buffer.

8. The method of claim 1, wherein said template of step b) is circular.

9. The method of claim 7, wherein said substantially self-ligated products are used in said amplifying step.

10. The method of claim 1, wherein said amplifying step uses a polymerase.

11. The method of claim 10, wherein said polymerase is phi29 polymerase.

12. The method of claim 4, wherein the mechanical means comprises use of a shearing means.

13. The method of claim 1, wherein the organism comprises uncultured organism.

14. The method of claim 1, wherein the at least one organism is derived from an environmental sample

15. The method of claim 1, wherein the at least one organism is derived from a contaminated environmental sample.

16. The method of claim 1, wherein the organisms comprise a mixture of terrestrial microorganisms or marine microorganisms, or a mixture of terrestrial microorganisms and marine microorganisms.

17. The method of claim 1, wherein the organism is an extremophile.

18. The method of claim 5, wherein the extremophile comprises one or more organisms selected from the group consisting of thermophiles, hyperthermophiles, psychrophiles, psychrotrophs, halophiles, alkalophiles, and acidophiles.

19. The method of claim 1, wherein the cDNA or genomic fragments comprise at least an operon, or portions thereof, of the donor microorganisms.

20. The method of claim 7, wherein the operon encodes a complete or partial metabolic pathway.

21. The method of claim 1, wherein said amplifying step is repeated.

22. A method for amplifying a DNA template from trace amounts of DNA derived from at least one species of organism comprising:
a) obtaining trace amounts of cDNA, gDNA, or genomic DNA fragments from at least one species of organisms;
b) preparing a circular template from said cDNA, gDNA, or genomic DNA fragments; and
c) amplifying the template.

23. A method for making a DNA template from trace amounts of DNA isolated from trace amounts of DNA from a mixed population of uncultivated cells comprising:
a) encapsulating individually, in a microenvironment, a plurality of cells from a mixed population of uncultivated cells;
b) creating a template from said cDNA, gDNA, or genomic DNA fragments; and
c) amplifying the template.

24. The method of claim 23, wherein said template is fragmented.

25. The method of claim 23, wherein said trace amounts of cDNA, gDNA, or genomic DNA fragments are partially or completely digested.

26. The method of claim 23, wherein the template fragmentation is achieved by enzymatic, chemical, photometric, mechanical or any means that provides segments.

27. The method of claim 22, wherein the enzymatic fragmentation comprises use of a DNAse or a restriction enzyme.

28. The method of claim 26, further comprising filling DNA ends by polymerase extension.

29. The method of claim 23, wherein said template is diluted to a degree sufficient to obtain substantially self-ligated products in the presence of ligase and ligase buffer.

30. The method of claim 29, wherein said substantially self-ligated products are used in said amplifying step.

31. The method of claim 20, wherein said amplifying step uses a polymerase.

32. The method of claim 31, wherein said polymerase is phi29 polymerase.

33. The method of claim 26, wherein the mechanical means comprises use of a shearing means.

34. The method of claim 23, wherein the organism is derived from an environmental sample.

35. The method of claim 23, wherein the organism is derived from a contaminated environmental sample.

36. The method of claim 20, wherein the organism is an extremophile.

37. The method of claim 31, wherein the extremophile comprises one or more organisms selected from the group consisting of thermophiles, hyperthermophiles, psychrophiles, psychrotrophs, halophiles, alkalophiles, and acidophiles.

38. The method claim 23, wherein said microenvironment has trace amounts of cells from at least one species of organism.

39. The method of claims 1, 22, or 23, wherein said amplifying step is performed by polymerase amplification.

40. The method of claim 39, wherein said amplifying step is performed by multiple displacement amplification (MDA).
